(19) United States
(12) Patent Application Publication
(10) Pub. No.: US 2026/0028331 A1
(43) Pub. Date: Jan. 29, 2026

(54) Methods And Intermediates for Preparing 2-[(4-{6-[(4-Cyano-2-fluorobenzyl)oxy]pyridin-2-yl}piperidin-1-yl)methyl]-1-[(2S)-oxetan-2-ylmethyl]-1H-benzimidazole-6-carboxylic acid, 1,3-Dihydroxy-2-(hydroxymethyl)propan-2-amine Salt

(71) Applicant: Pfizer Inc., New York, NY (US)

(72) Inventors: Matthew Badland, Sandwich, Kent (GB); Laura Jane Elizabeth Carson, Sandwich, Kent (GB); James Clarke, Sandwich, Kent (GB); Amelie Dion, Fanwood, NJ (US); Steven James Fussell, Sandwich, Kent (GB); Adam Edward Sanji Gymer, Sandwich, Kent (GB); Daniel Andrew Laity, Sandwich, Kent (GB); Jinu Suju Mathew, Sandwich, Kent (GB); Laura Caroline McGivern, Sandwich, Kent (GB); James Alan Mills, Sandwich, Kent (GB); Ian Brian Moses, Sandwich, Kent (GB); Benjamin Alan Pibworth, Sandwich, Kent (GB); James Rellegue, Sandwich, Kent (GB); Adam John Reynolds-Scott, Sandwich, Kent (GB); Emily Kay Rose, Sandwich, Kent (GB); Vaclav Svoboda, Sandwich, Kent (GB); Adam Sebastian West, Sandwich, Kent (GB); Chloe Joy White, Sandwich, Kent (GB)

(73) Assignee: Pfizer Inc., New York, NY (US)

(21) Appl. No.: 18/993,920

(22) PCT Filed: Jul. 19, 2023
(86) PCT No.: PCT/IB2023/057360

§ 371 Date: Jan. 13, 2025

Related U.S. Application Data
(60) Provisional application No. 63/391,459, filed on Jul. 22, 2022.

(51) Int. Cl. C07D 405/14; C07D 401/04

(52) U.S. Cl. CPC C07D 405/14; C07D 401/04

(57) ABSTRACT

The invention provides methods and certain intermediates for preparing 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid 1,3-dihydroxy-2-(hydroxymethyl) propan-2-amine salt, and processes for preparing these intermediates.

### FIELD OF INVENTION

The invention provides methods and certain intermediates for preparing 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid, 1,3-dihydroxy-2-(hydroxymethyl) propan-2-amine salt, and processes for preparing these intermediates.

### BACKGROUND OF THE INVENTION

Diabetes is a major public health concern because of its increasing prevalence and associated health risks. The disease is characterized by high levels of blood glucose resulting from defects in insulin production, insulin action, or both. Two major forms of diabetes are recognized, Type 1 and Type 2. Type 1 diabetes (T1D) develops when the body's immune system destroys pancreatic beta cells, the only cells in the body that make the hormone insulin that regulates blood glucose. To survive, people with Type 1 diabetes must have insulin administered by injection or a pump. Type 2 diabetes mellitus (referred to generally as T2DM) usually begins with either insulin resistance or when there is insufficient production of insulin to maintain an acceptable glucose level.

Currently, various pharmacological approaches are available for treating hyperglycemia and subsequently, T2DM (Hampp, C. et al. Use of Antidiabetic Drugs in the U.S., 2003-2012, Diabetes Care 2014, 37, 1367-1374). These may be grouped into six major classes, each acting through a different primary mechanism: (A) Insulin secretogogues, including sulphonyl-ureas (e.g., glipizide, glimepiride, glyburide), meglitinides (e.g., nateglidine, repaglinide), dipeptidyl peptidase IV (DPP-IV) inhibitors (e.g., sitagliptin, vildagliptin, alogliptin, dutogliptin, linagliptin, saxogliptin), and glucagon-like peptide-1 receptor (GLP-1R) agonists (e.g., liraglutide, albiglutide, exenatide, lixisenatide, dulaglutide, semaglutide), which enhance secretion of insulin by acting on the pancreatic beta-cells. Sulphonyl-ureas and meglitinides have limited efficacy and tolerability, cause weight gain and often induce hypoglycemia. DPP-IV inhibitors have limited efficacy. Marketed GLP-1R agonists are peptides administered by subcutaneous injection. Liraglutide is additionally approved for the treatment of obesity. (B) Biguanides (e.g., metformin) are thought to act primarily by decreasing hepatic glucose production. Biguanides often cause gastrointestinal disturbances and lactic acidosis, further limiting their use. (C) Inhibitors of alpha-glucosidase (e.g., acarbose) decrease intestinal glucose absorption. These agents often cause gastrointestinal disturbances. (D) Thiazolidinediones (e.g., pioglitazone, rosiglitazone) act on a specific receptor (peroxisome proliferator-activated receptor-gamma) in the liver, muscle, and fat tissues. They regulate lipid metabolism subsequently enhancing the response of these tissues to the actions of insulin. Frequent use of these drugs may lead to weight gain and may induce edema and anemia. (E) Insulin is used in more severe cases, either alone or in combination with the above agents, and frequent use may also lead to weight gain and carries a risk of hypoglycemia. (F) sodium-glucose linked transporter cotransporter 2 (SGLT2) inhibitors (e.g., dapagliflozin, empagliflozin, canagliflozin, ertugliflozin) inhibit reabsorption of glucose in the kidneys and thereby lower glucose levels in the blood. This emerging class of drugs may be associated with ketoacidosis and urinary tract infections.

However, with the exception of GLP-1R agonists and SGLT2 inhibitors, the drugs have limited efficacy and do not address the most important problems, the declining B-cell function and the associated obesity.

Obesity is a chronic disease that is highly prevalent in modern society and is associated with numerous medical problems including hypertension, hypercholesterolemia, and coronary heart disease. It is further highly correlated with T2DM and insulin resistance, the latter of which is generally accompanied by hyperinsulinemia or hyperglycemia, or both. In addition, T2DM is associated with a two to fourfold increased risk of coronary artery disease. Presently, the only treatment that eliminates obesity with high efficacy is bariatric surgery, but this treatment is costly and risky. Pharmacological intervention is generally less efficacious and associated with side effects. There is therefore an obvious need for more efficacious pharmacological intervention with fewer side effects and convenient administration.

Although T2DM is most commonly associated with hyperglycemia and insulin resistance, other diseases associated with T2DM include hepatic insulin resistance, impaired glucose tolerance, diabetic neuropathy, diabetic nephropathy, diabetic retinopathy, obesity, dyslipidemia, hypertension, hyperinsulinemia, and nonalcoholic fatty liver disease (NAFLD).

NAFLD is the hepatic manifestation of metabolic syndrome, and is a spectrum of hepatic conditions encompassing steatosis, non-alcoholic steatohepatitis (NASH), fibrosis, cirrhosis and ultimately hepatocellular carcinoma. NAFLD and NASH are considered the primary fatty liver diseases as they account for the greatest proportion of individuals with elevated hepatic lipids. The severity of NAFLD/NASH is based on the presence of lipid, inflammatory cell infiltrate, hepatocyte ballooning, and the degree of fibrosis. Although not all individuals with steatosis progress to NASH, a substantial portion does.

GLP-1 is a 30 amino acid long incretin hormone secreted by the L-cells in the intestine in response to ingestion of food. GLP-1 has been shown to stimulate insulin secretion in a physiological and glucose-dependent manner, decrease glucagon secretion, inhibit gastric emptying, decrease appetite, and stimulate proliferation of beta-cells. In non-clinical experiments GLP-1 promotes continued beta-cell competence by stimulating transcription of genes important for glucose-dependent insulin secretion and by promoting beta-cell neogenesis (Meier, et al. Biodrugs. 2003; 17 (2): 93-102).

In a healthy individual, GLP-1 plays an important role regulating post-prandial blood glucose levels by stimulating glucose-dependent insulin secretion by the pancreas resulting in increased glucose absorption in the periphery. GLP-1 also suppresses glucagon secretion, leading to reduced hepatic glucose output. In addition, GLP-1 delays gastric emptying and slows small bowel motility delaying food absorption. In people with T2DM, the normal post-prandial rise in GLP-1 is absent or reduced (Vilsboll T, et al. Diabetes. 2001. 50; 609-613).

Holst (Physiol. Rev. 2007, 87, 1409) and Meier (Nat. Rev. Endocrinol. 2012, 8, 728) describe that GLP-1 receptor agonists, such as GLP-1, liraglutide and exendin-4, have 3 major pharmacological activities to improve glycemic control in patients with T2DM by reducing fasting and postprandial glucose (FPG and PPG): (i) increased glucose-dependent insulin secretion (improved first- and second-phase), (ii) glucagon suppressing activity under hyperglycemic conditions, (iii) delay of gastric emptying rate resulting in retarded absorption of meal-derived glucose.

There remains a need for an easily-administered prevention and/or treatment for cardiometabolic and associated diseases.

The compound 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid is a GLP-1R agonist described in U.S. Pat. No. 10,208,019 (see Example 4A-01 of the patent), the disclosure of which is hereby incorporated by reference herein in its entirety for all purposes. The compound, which is herein designated as “C111”, has the following structure:

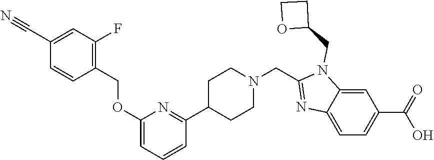 [SMILES: N#Cc1ccc(COc2cccc(C3CCN(Cc4nc5ccc(C(=O)O)cc5n4C[C@@H]4CCO4)CC3)n2)c(F)c1]

The compound may be administered in the form of a pharmaceutically acceptable salt thereof, e.g. as its 1,3-dihydroxy-2-(hydroxymethyl) propan-2-amine salt (also known as its 2-amino-2-(hydroxymethyl) propane-1,3-diol salt) or as its tris(hydroxyethyl)methylamine salt, or its tris salt].

The tris salt of 2-[(4-{6-[(4-Cyano-2-fluorobenzyl)oxy]pyridin-2-yl}piperidin-1-yl)methyl]-1-[(2S)-oxetan-2-ylmethyl]-1H-benzimidazole-6-carboxylic acid means a salt of C111 made by using 1,3-dihydroxy-2-(hydroxymethyl) propan-2-amine. The tris is associated with the carboxylic acid moiety of C111. Unless otherwise stated, when referencing the tris salt of C111, the counterion and C111 are in a stoichiometric ratio of about 1:1 (i.e. from 0.9:1.0 to 1.0:0.9, for example, from 0.95:1.00 to 1.00:0.95, or from 0.99:1.00 to 1.00:1.01). Another chemical name for tris salt of C111 is 1,3-dihydroxy-2-(hydroxymethyl) propan-2-aminium 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylate, which can also be represented, for example, by one of the following structures.

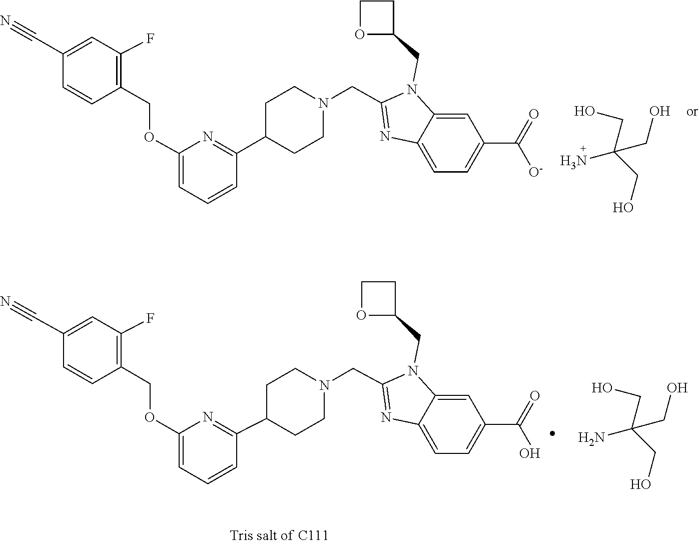 [SMILES: N#Cc1ccc(COc2cccc(C3CCN(Cc4nc5ccc(C(=O)[O-])cc5n4C[C@@H]4CCO4)CC3)n2)c(F)c1 ; [NH3+]C(CO)(CO)CO ; N#Cc1ccc(COc2cccc(C3CCN(Cc4nc5ccc(C(=O)O)cc5n4C[C@@H]4CCO4)CC3)n2)c(F)c1 ; NC(CO)(CO)CO]

It is well known that a solid form, for example a crystalline form of a particular drug (including, e.g., anhydrate, hydrate, solvate, etc.) is often an important determinant of the drug's ease of preparation, stability, solubility, storage stability, ease of formulation, ease of handling, and in vivo pharmacology and/or efficacy. Different crystalline forms occur where the same composition of matter crystallizes in a different lattice arrangement resulting in different thermodynamic properties and stabilities specific to the particular polymorph form. In cases where two or more solid forms (e.g. two or more crystalline forms, or an amorphous form and one or more crystalline forms) can be produced, it is desirable to have a method to make each of the solid forms in pure form. In deciding which solid form is preferable, the numerous properties of the solid forms must be compared, and the preferred solid (e.g. crystalline) form chosen based on the many physical property variables. It is entirely possible that one crystalline form can be preferable in some circumstances where certain aspects such as ease of preparation, stability, etc. are deemed to be critical. In other situations, a different crystalline form maybe preferred for greater solubility and/or superior pharmacokinetics. Moreover, because of the potential advantages associated with one pure crystalline form, it is desirable to prevent or minimize polymorphic conversion (i.e., conversion of one crystal form to another; or conversion between one crystal form and amorphous form) when two or more solid forms of one substance can exist. Such polymorphic conversion can occur during both the preparation of formulations containing a solid form (e.g. a crystalline form), and during storage of a pharmaceutical dosage form containing a solid form (e.g. a crystalline form). Because improved drug formulations showing, for example, better bioavailability or better stability are consistently sought, there is an ongoing need for new or purer solid (e.g. crystalline) forms of existing drug molecules. Moreover, there is an ongoing need for cheaper and/or more efficient processes for making existing drug molecules (including those improving purity, and/or producing less undesired impurities). The processes and intermediates described herein are directed toward this and other important ends.

### SUMMARY OF THE INVENTION

In one embodiment (Embodiment A1), the present invention provides a process for preparing the bis(4-methylbenzenesulfonate) salt of 3-fluoro-4-(((6-(piperidin-4-yl) pyridin-2-yl)oxy)methyl)benzonitrile,

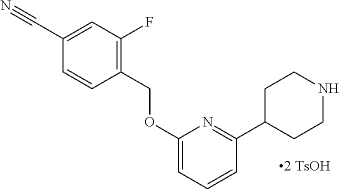 [SMILES: N#Cc1ccc(COc2cccc(C3CCNCC3)n2)c(F)c1]

which process comprises:
(a1) reacting tert-butyl 4-(6-chloropyridin-2-yl) piperidine-1-carboxylate with 3-fluoro-4-(hydroxymethyl)benzonitrile in the presence a palladium catalyst [such as palladium (II) acetate, tris(dibenzylideneacetone) dipalladium, or palladium (II) chloride], a base [such as an inorganic base, for example, potassium phosphate tribasic, cesium carbonate, potassium hydroxide, or sodium hydride], and a phosphorous ligand [e.g. a monodentate phosphorous ligand or a bidentate phosporous ligand, such as 2-dicyclohexylphosphino-2′,4′,6′-triisopropylbiphenyl (X-Phos), (2-biphenylyl)di-tert-butylphosphine (JohnPhos), 5-(di-tert-butylphosphino)-1′,3′,5′-triphenyl-1′H-[1,4′]bipyrazole (Bippyphos), 5-[bis(1-adamantyl)phosphino]-1′,3′,5′-triphenyl-1,4′-bi-1H-pyrazole (AdBippyphos), 2-(dicyclohexylphosphino) 3,6-dimethoxy-2′,4′,6′-triisopropyl-1,1′-biphenyl (BrettPhos), di(adamantan-1-yl) (2′,4′,6′-triisopropyl-3,6-dimethoxy-2-biphenyl Yl)phosphine (AdBrettPhos), 2-dicyclohexylphosphino-2′,6-bis(N,N-dimethylamino) biphenyl (CPhos), 2-dicyclohexylphosphino-2′,6′-dimethoxybiphenyl (SPhos), 2-dicyclohexylphosphino-2′-(N,N-dimethylamino) biphenyl (DavePhos), 2-di-tert-butylphosphino-2′-(N,N-dimethylamino) biphenyl (tBuDavePhos), 2-(di-tert-butylphosphino)-2′,4′,6′-triisopropyl-3,6-dimethoxy-1,1′-biphenyl (tBuBrettPhos), di-tert-butyl (2′,4′,6′-triisopropyl-3-methoxy-6-methyl-[1,1′-biphenyl]-2-yl)phosphine (RockPhos), 4,5-bis(diphenylphosphino)-9,9-dimethylxanthene (XantPhos), or bis[(2-diphenylphosphino)phenyl] ether (DPEPhos)], in a solvent system [such as comprising anisole, acetonitrile, tetrahydrofuran, dioxane, or methyl tert-butyl ether], to form tert-butyl 4-(6-((4-cyano-2-fluorobenzyl)oxy)pyridin-2-yl) piperidine-1-carboxylate, wherein the amount of the 3-fluoro-4-(hydroxymethyl)benzonitrile is about 1.0 to about 1.1 molar equivalents to the tert-butyl 4-(6-chloropyridin-2-yl) piperidine-1-carboxylate;
(a2) adding ethanol to the reaction mixture from Step (a1);
(a3) filtering the resultant reaction mixture from Step (a2);
(b1) adding p-toluenesulfonic acid monohydrate to the filtrate from Step (a3), thereby reacting the tert-butyl 4-(6-((4-cyano-2-fluorobenzyl)oxy)pyridin-2-yl) piperidine-1-carboxylate with the p-toluenesulfonic acid monohydrate, to form a bis(4-methylbenzenesulfonate) salt of 3-fluoro-4-(((6-(piperidin-4-yl) pyridin-2-yl)oxy)methyl)benzonitrile, wherein the amount of the p-toluenesulfonic acid monohydrate is about 2.0 to about 3.0 molar equivalents to the tert-butyl 4-(6-((4-cyano-2-fluorobenzyl)oxy)pyridin-2-yl) piperidine-1-carboxylate; and
(b2) isolating the bis(4-methylbenzenesulfonate) salt of 3-fluoro-4-(((6-(piperidin-4-yl) pyridin-2-yl)oxy)methyl)benzonitrile from Step (b1).

In one embodiment (Embodiment B1), the present invention provides a process for preparing bis(4-methylbenzenesulfonate) salt of 3-fluoro-4-(((6-(piperidin-4-yl) pyridin-2-yl)oxy)methyl)benzonitrile,

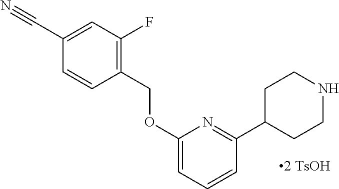 [SMILES: N#Cc1ccc(COc2cccc(C3CCNCC3)n2)c(F)c1]

which process comprising:
(a1) reacting tert-butyl 4-(6-chloropyridin-2-yl) piperidine-1-carboxylate with 3-fluoro-4-(hydroxymethyl)benzonitrile in the presence a palladium catalyst [such as palladium (II) acetate, tris (dibenzylideneacetone) dipalladium, or palladium (II) chloride], a base [such as an inorganic base for example potassium phosphate tribasic, cesium carbonate, potassium hydroxide, or sodium Hydride], and a phosphorous ligand [such as a monodentate phosphorous ligand or a bidentate phosphorous ligand, for example, 2 dicyclohexylphosphino-2′,4′,6′-triisopropylbiphenyl (X Phos), (2 bBiphenylyl)di tert butylphosphine (JohnPhos), 5-(di-tert butylphosphino) 1′,3′,5′ triphenyl-1′H-[1,4′]bipyrazole (Bippyphos), 5-[bis (1 adamantyl)phosphino]-1′,3′,5′-triphenyl-1,4′-bi-1H-pyrazole (AdBippyphos), 2-(dicyclohexylphosphino)-3,6-dimethoxy-2′,4′,6′-triisopropyl-1,1′-biphenyl (BrettPhos), di(adamantan-1-yl) (2′,4′,6′-triisopropyl-3,6-dimethoxy-2-biphenyl (CPhos), 2-dicyclohexylphosphino-2′,6′-dimethoxybiphenyl (SPhos), 2-dicyclohexylphosphino-2′-(N,N-dimethylamino) biphenyl (DavePhos), 2-di-tert-butylphosphino-2′-(N,N-dimethylamino) biphenyl (tBuDavePhos), 2-(di-tert-butylphosphino)-2′,4′,6′-triisopropyl-3,6-dimethoxy-1,1′-biphenyl (tBuBrettPhos), di-tert-butyl (2′,4′,6′-triisopropyl-3-methoxy-6-methyl-[1,1′-biphenyl]-2-yl)phosphine (RockPhos), 4,5-bis(diphenylphosphino)-9,9-dimethylxanthene (XantPhos), or bis[(2-diphenylphosphino)phenyl] ether (DPEPhos)], in a solvent system [such as one comprising anisole, acetonitrile, tetrahydrofuran, dioxane, or methyl tert-butyl ether], to form tert-butyl 4-(6-((4-cyano-2-fluorobenzyl)oxy)pyridin-2-yl) piperidine-1-carboxylate, wherein the amount of the 3-fluoro-4-(hydroxymethyl)benzonitrile is about 1.0 to about 1.1 molar equivalents to the tert-butyl 4-(6-chloropyridin-2-yl) piperidine-1-carboxylate;
(a2) upon reaction completion in Step (a1), adding water, ethyl acetate, and ethanol to the reaction mixture;
(a3) separating the organic phase from Step (a2) from the aqueous phase;
(b1) adding p-toluenesulfonic acid monohydrate to the separated organic phase from Step (a3), thereby reacting the tert-butyl 4-(6-((4-cyano-2-fluorobenzyl)oxy)pyridin-2-yl) piperidine-1-carboxylate with the p-toluenesulfonic acid monohydrate, to form bis(4-methylbenzenesulfonate) salt of 3-fluoro-4-(((6-(piperidin-4-yl) pyridin-2-yl)oxy)methyl)benzonitrile, wherein the amount of the p-toluenesulfonic acid monohydrate is about 2.0 to about 3.0 molar equivalents to the tert-butyl 4-(6-((4-cyano-2-fluorobenzyl)oxy)pyridin-2-yl) piperidine-1-carboxylate; and
(b2) isolating the bis(4-methylbenzenesulfonate) salt of 3-fluoro-4-(((6-(piperidin-4-yl) pyridin-2-yl)oxy)methyl)benzonitrile from Step (b1).

In one embodiment (Embodiment C1), the present invention provides a process for preparing bis(4-methylbenzenesulfonate) salt of 3-fluoro-4-(((6-(piperidin-4-yl) pyridin-2-yl)oxy)methyl)benzonitrile,

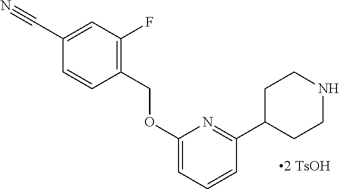 [SMILES: N#Cc1ccc(COc2cccc(C3CCNCC3)n2)c(F)c1]

which process comprising:
(a1) reacting tert-butyl 4-(6-chloropyridin-2-yl) piperidine-1-carboxylate with 3-fluoro-4-(hydroxymethyl)benzonitrile in the presence of a copper catalyst [such as copper (I) iodide, copper (I) acetate, tetrakisacetonitrile copper (I) triflate, tetrakisacetonitrile copper (I) hexafluorophosphate, or copper trifluoromethanesulfonate], a base [such as sodium tert-pentoxide, cesium carbonate, potassium phosphate, potassium hexamethylenedisilazide, or sodium tert-butoxide], and a ligand [such as N,N′-bis(1-naphthylmethyl) oxamide or N,N′-diphenylethyloxalamide], in a solvent system [such as one comprising 1,4-dioxane, anisole, methyl tert-butyl ether (MTBE), or 2-methyl-tetrahydrofuran (2-MeTHF)], to form tert-butyl 4-(6-((4-cyano-2-fluorobenzyl)oxy)pyridin-2-yl) piperidine-1-carboxylate, wherein the amount of the 3-fluoro-4-(hydroxymethyl)benzonitrile is about 1.1 to about 1.3 (e.g. 1.2) molar equivalents to the tert-butyl 4-(6-chloropyridin-2-yl) piperidine-1-carboxylate;
(a2) filtering the reaction mixture from Step (a1), wherein the filtering further comprises washing with methyl tert-butyl ether (MTBE);
(a3) concentrating the filtrate from Step (2) to obtain tert-butyl 4-(6-((4-cyano-2-fluorobenzyl)oxy)pyridin-2-yl) piperidine-1-carboxylate;
(b1) dissolving the tert-butyl 4-(6-((4-cyano-2-fluorobenzyl)oxy)pyridin-2-yl) piperidine-1-carboxylate from Step (a3) in a solvent system comprising 1,4-dioxane and MTBE to form a solution; and then adding p-toluenesulfonic acid monohydrate to the solution, thereby reacting the tert-butyl 4-(6-((4-cyano-2-fluorobenzyl)oxy)pyridin-2-yl) piperidine-1-carboxylate with the p-toluenesulfonic acid monohydrate, to form bis(4-methylbenzenesulfonate) salt of 3-fluoro-4-(((6-(piperidin-4-yl) pyridin-2-yl)oxy)methyl)benzonitrile, wherein the amount of the p-toluenesulfonic acid monohydrate is about 2.0 to about 2.5 (e.g. about 2.1) molar equivalents to the tert-butyl 4-(6-((4-cyano-2-fluorobenzyl)oxy)pyridin-2-yl) piperidine-1-carboxylate; and
(b2) isolating the bis(4-methylbenzenesulfonate) salt of 3-fluoro-4-(((6-(piperidin-4-yl) pyridin-2-yl)oxy)methyl)benzonitrile from Step (b1).

In one embodiment (Embodiment D1), the present invention provides an intermediate useful for preparing C111 or tris salt of C111, which is:
anhydrous 3-fluoro-4-(((6-(piperidin-4-yl) pyridin-2-yl)oxy)methyl)benzonitrile bis(4-methylbenzenesulfonate) salt;
monohydrate of 3-fluoro-4-(((6-(piperidin-4-yl) pyridin-2-yl)oxy)methyl)benzonitrile bis(4-methylbenzenesulfonate) salt;
mono tosylate salt of 3-fluoro-4-(((6-(piperidin-4-yl) pyridin-2-yl)oxy)methyl)benzonitrile;
bis mesylate salt of 3-fluoro-4-(((6-(piperidin-4-yl) pyridin-2-yl)oxy)methyl)benzonitrile;
mono mesylate salt of 3-fluoro-4-(((6-(piperidin-4-yl) pyridin-2-yl)oxy)methyl)benzonitrile;
mono sulfate salt of 3-fluoro-4-(((6-(piperidin-4-yl) pyridin-2-yl)oxy)methyl)benzonitrile;
hemi sulfate salt of 3-fluoro-4-(((6-(piperidin-4-yl) pyridin-2-yl)oxy)methyl)benzonitrile;
a crystalline methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate; or
hemi-barium salt of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid.

In one embodiment (Embodiment E1), the present invention provides a process for preparing methyl(S)-2-(chloromethyl)-1-(oxetan-2-ylmethyl)-1H-benzo[d]imidazole-6-carboxylate

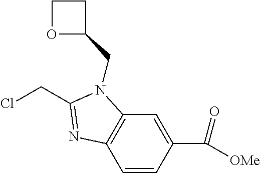 [SMILES: COC(=O)c1ccc2nc(CCl)n(C[C@@H]3CCO3)c2c1]

which process comprising:
(a1) providing methyl(S)-4-amino-3-(oxetane-2-carboxamido)benzoate;
(a2) reducing the methyl(S)-4-amino-3-(oxetane-2-carboxamido)benzoate in the presence of a reducing reagent to form methyl(S)-4-amino-3-((oxetan-2-ylmethyl)amino) benzoate; and
(a3) reacting methyl(S)-4-amino-3-((oxetan-2-ylmethyl)amino)benzoate with 2-chloro-1,1,1-trimethoxyethane in the presence of an acid (e.g. an organic acid such as citric acid or p-toluenesulfonic acid) to form methyl(S)-2-(chloromethyl)-1-(oxetan-2-ylmethyl)-1H-benzo[d]imidazole-6-carboxylate.

In one embodiment (Embodiment F1), the present invention provides a process for preparing methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate,

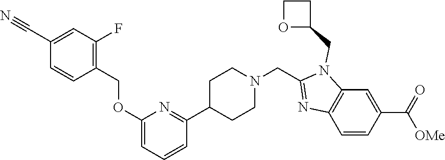 [SMILES: COC(=O)c1ccc2nc(CN3CCC(c4cccc(OCc5ccc(C#N)cc5F)n4)CC3)n(C[C@@H]3CCO3)c2c1]

which process comprising:
(a1) reacting methyl(S)-2-(chloromethyl)-1-(oxetan-2-ylmethyl)-1H-benzo[d]imidazole-6-carboxylate with bis(4-methylbenzenesulfonate) salt of 3-fluoro-4-(((6-(piperidin-4-yl) pyridin-2-yl)oxy)methyl)benzonitrile in the presence diisopropylethylamine in a solvent system comprising acetonitrile, to form the methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate, wherein the amount of the bis(4-methylbenzenesulfonate) salt of 3-fluoro-4-(((6-(piperidin-4-yl) pyridin-2-yl)oxy)methyl)benzonitrile is about 1.1 to about 1.5 molar equivalents to the methyl(S)-2-(chloromethyl)-1-(oxetan-2-ylmethyl)-1H-benzo[d]imidazole-6-carboxylate, and wherein the amount of the diisopropylethylamine is about 4.0 to about 6.0 (e.g. 5.0) molar equivalents to the methyl(S)-2-(chloromethyl)-1-(oxetan-2-ylmethyl)-1H-benzo[d]imidazole-6-carboxylate.

In one embodiment (Embodiment G1), the present invention provides a process for preparing methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate,

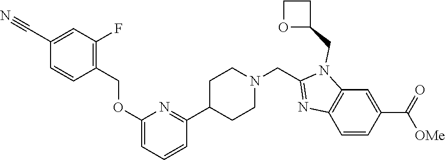 [SMILES: COC(=O)c1ccc2nc(CN3CCC(c4cccc(OCc5ccc(C#N)cc5F)n4)CC3)n(C[C@@H]3CCO3)c2c1]

which process comprising:
(a1) reacting methyl(S)-2-(chloromethyl)-1-(oxetan-2-ylmethyl)-1H-benzo[d]imidazole-6-carboxylate with bis(4-methylbenzenesulfonate) salt of 3-fluoro-4-(((6-(piperidin-4-yl) pyridin-2-yl)oxy)methyl)benzonitrile in the presence diisopropylethylamine in a solvent system comprising methanol, to form the methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate, wherein the amount of the bis(4-methylbenzenesulfonate) salt of 3-fluoro-4-(((6-(piperidin-4-yl) pyridin-2-yl)oxy)methyl)benzonitrile is about 1.0 to about 1.2 (e.g. 1.1) molar equivalents to the methyl(S)-2-(chloromethyl)-1-(oxetan-2-ylmethyl)-1H-benzo[d]imidazole-6-carboxylate, and wherein the amount of the diisopropylethylamine is about 4.0 to about 6.0 (e.g. 5.0) molar equivalents to the methyl(S)-2-(chloromethyl)-1-(oxetan-2-ylmethyl)-1H-benzo[d]imidazole-6-carboxylate.

In one embodiment (Embodiment H1), the present invention provides a process for preparing hemi-barium salt of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid

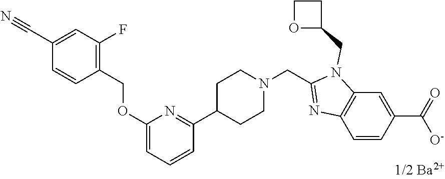 [SMILES: N#Cc1ccc(COc2cccc(C3CCN(Cc4nc5ccc(C(=O)[O-])cc5n4C[C@@H]4CCO4)CC3)n2)c(F)c1]

which process comprising:
(a1) reacting methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate with barium hydroxide [Ba(OH)₂] in a solvent system comprising an organic solvent and water, to form hemi-barium salt of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid, wherein the amount of barium hydroxide [Ba(OH)₂] is about 0.5 to about 0.6 (e.g. about 0.5) molar equivalent to the methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate; and
(a2) optionally isolating the hemi-barium salt of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid from Step (a1).

In one embodiment (Embodiment J1), the present invention provides a process for preparing tris salt of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid,

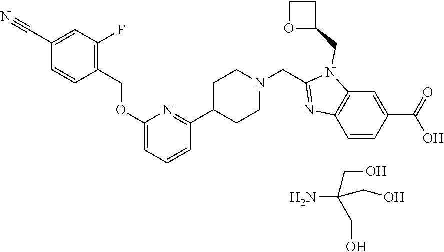

which process comprising:
(a1) reacting methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate with barium hydroxide [Ba(OH)₂] in a solvent system comprising acetonitrile and water, to form hemi-barium salt of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid, wherein the amount of barium hydroxide [Ba(OH)₂] is about 0.5 to about 0.6 (e.g. about 0.5) molar equivalent to the methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate;
(a2) adding water, a water-immiscible organic solvent (e.g. toluene, TBME, or Ethyl acetate), and an organic acid (e.g. acetic acid) to the reaction mixture from Step (a1), and mixing the resultant mixture to form(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid;
(a3) separating the organic phase/solution from the aqueous phase/solution from the mixture from Step (a2);
(a4) adding 2-amino-2-(hydroxymethyl)-1,3-propanediol to the separated organic phase from Step (a3) whereby reacting 2-amino-2-(hydroxymethyl)-1,3-propanediol and the(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid, to form tris salt of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid, wherein the reaction mixture is held at a holding temperature of about 35° C. to about 55° C. (e.g. about 40° C. to about 50° C., or about 45° C.);
(a5) adding a seed crystalline material of tris salt of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid to the reaction mixture from Step (a4) to form a slurry, wherein the slurry is held at the holding temperature for a period of time of greater than about 1 minute;
(a6) cooling the slurry from Step (a5) to a temperature of about 20° C. to about 30° C. (e.g. 25° C.), and holding the slurry at that temperature for a period of time of greater than about 1 minute;
(a7) optionally heating the slurry from Step (a6) to the holding temperature [about 40° C. to about 50° C., e.g. about 45° C.] and holding the slurry at the holding temperature for a period of time of greater than about 1 minute;
(a8) optionally cooling the slurry from Step (a7) to a temperature of about 15° C. to about 25° C. (e.g. 20° C.), and holding the slurry at that temperature for a period of time of greater than about 1 minute; and
(a9) isolating the tris salt of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid in the slurry from Step (a6) or (a8).

In one embodiment (Embodiment K1), the present invention provides a process for preparing tris salt of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid

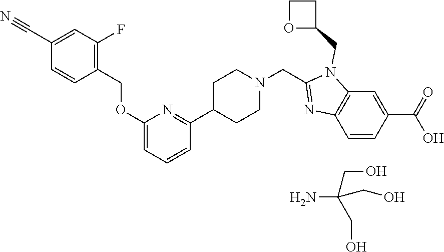

which process comprising:
(a1) reacting methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate with barium hydroxide [Ba(OH)₂] in a solvent system comprising acetone and water, to form hemi-barium salt of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid, wherein the amount of barium hydroxide [Ba(OH)₂] is about 0.5 to about 0.6 (e.g. about 0.5) molar equivalent to the methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate;
(a2) adding water, a water-immiscible organic solvent (e.g. toluene, TBME, or Ethyl acetate), and an organic acid (e.g. acetic acid) to the reaction mixture from Step (a1), and mixing the resultant mixture to form(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylic acid;
(a3) separating the organic phase from the aqueous phase from the mixture from Step (a2) and optionally adding methanol to the separated organic phase;
(a4) adding 2-amino-2-(hydroxymethyl)-1,3-propanediol to the separated organic phase from Step (a3) whereby reacting 2-amino-2-(hydroxymethyl)-1,3-propanediol and the(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid, to form tris salt of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid, wherein the reaction mixture is held at a holding temperature of about 35° C. to about 55° C. (e.g. about 40° C. to about 50° C., or about 45° C.);
(a5) adding a seed crystalline material of tris salt of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid to the reaction mixture from Step (a4) to form a slurry, wherein the slurry is held at the holding temperature for a period of time of greater than about 1 minute;
(a6) cooling the slurry from Step (a5) to a temperature of about 15° C. to about 20° C. (e.g. 20° C.), and holding the slurry at that temperature for a period of time of greater than about 1 minute;
(a7) isolating the tris salt of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid in the slurry from Step (a6).

In one embodiment (Embodiment L1), the present invention provides a method for preparing Form 1 of tris salt of 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid, which method comprises:
(a) suspending tris salt of 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid in a solvent system, wherein the solvent system consists of dimethyl sulfoxide (DMSO) and water, wherein the volume ratio of DMSO:water is from about 10:1 to about 6:1 (e.g., from about 9:1 to about 7:1, or about 8:1), and wherein the ratio of the water (volume) to the tris salt of 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid (weight) is from about 0.8 mL/g to about 1.2 mL/g (e.g. 1.0 mL/g) at a temperature of from about 20° C. to about 35° C. (e.g. from about 20° C. to about 30° C.);
(b) heating the suspension in Step (a) to an elevated temperature from about 60° C. to about 70° C. (e.g. about 65° C.) to form a solution, and then mixing the solution at the elevated temperature for a period of time of greater than about 1 minute;
(c) adding water to the solution from Step (b) slowly while maintaining the reaction mixture as a solution, wherein the amount of water added is about the same as the water used in Step (a), and then holding the resultant solution at the elevated temperature for a period of time of greater than about 1 minute;
(d) seeding the solution with a crystalline Form 1 material of tris salt of 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid while maintaining the temperature at the elevated temperature, wherein the amount of the seed crystalline is about 0.5 weight % or more (e.g. about 0.5 weight % or about 1.0 weight %) of the tris salt of 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid used in Step (a), and then holding the resultant mixture at the elevated temperature for a period of time of greater than about 1 minute;
(e) optionally cooling the temperature of the mixture from Step (d) to about 30° C. slowly, and then holding the mixture at that temperature for a period of time of greater than about 1 minute;
(f) optionally heating the mixture from Step (e) to about 40° C. to about 50° C. slowly, and then holding the mixture at that temperature for a period of time of greater than about 1 minute;
(g) cooling the temperature of the mixture from Step (d) or Step (f) [if Steps (e) and (f) are carried out] to about 15° C. slowly, and then holding the mixture at that temperature for a period of time of greater than about 1 minute; and
(h) isolating the solid from the resultant mixture from Step (g) to afford the Form I of tris salt of 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid.

In one embodiment (Embodiment M1), the present invention provides a method for preparing Form 1 of tris salt of 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid, which method comprises:
(a) suspending tris salt of 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid in a solvent system, wherein the solvent system consists of tetrahydrofuran (THF) and water, wherein the volume ratio of THF:water is from about 1:1 to about 4:1, (e.g. from about 2.5:1 to about 3.5:1, from about 2.8:1 to about 3.2:1, from about 2.9:1 to about 3.1:1, or about 3.0:1), and wherein the ratio of the water (volume) to the tris salt of 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid (weight) is from about 1.1 mL/g to about 3.8 mL/g (e.g. from about 1.3 mL/g to about 1.5 mL/g, or about 1.39 mL/g) at a temperature of from about 20° C. to about 35° C. (e.g. from about 20° C. to about 30° C., about 22° C.);
(b) heating the suspension in Step (a) to a high temperature of from about 49° C. to about 59° C. (e.g. about 55° C.) to form a solution, cooling the temperature to a holding temperature of from about 47° C. to about 51° C. (e.g. about 49° C.) while the mixture remains as a solution, and optionally mixing the solution at the holding temperature for a period of time of greater than about 1 minute;
(c) seeding the solution from Step (b) with a crystalline Form 1 material of tris salt of 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid while maintaining the temperature at the holding temperature, wherein the amount of the seed crystalline is about 0.5 weight % or more (e.g. about 0.5 weight % or about 1.0 weight %) of the tris salt of 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid used in Step (a), and then holding the resultant mixture at the holding temperature for a period of time of greater than about 1 minute;
(d) cooling the temperature of the mixture from Step (c) to an intermediate temperature of about 35° C. slowly, and then holding the mixture at the intermediate temperature for a period of time of greater than about 1 minute;
(e) adding a water-miscible organic solvent (e.g. acetonitrile, isopropanol, or acetone) to the mixture from Step (d) slowly while maintaining the temperature of the mixture at the intermediate temperature, and then holding the mixture at intermediate temperature for a period of time of greater than about 1 minute;
(f) cooling the temperature of the mixture from Step (e) to a low temperature of about 10° C. slowly, and then holding the mixture at the low temperature for a period of time of greater than about 1 minute;
(g) taking a sample of the mixture (slurry) to determine the particle size of the solid in the mixture of greater than about 1 minute;
(h) performing high shear wet milling until the D90 of the particle size of the solid in the mixture is less than about 150M; and
(i) isolating the solid from the resultant mixture from Step (f) to afford the Form I of tris salt of 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid.

### BRIEF DESCRIPTION OF FIGURES

FIG. 1 shows an observed powder X-ray diffraction pattern (PXRD) for Form 1 of bis-tosylate salt of C104 (anhydrous) carried out on a Bruker AXS D8 Endeavor diffractometer equipped with a Cu Kα radiation source (wavelength of 1.5406 Å).

FIG. 2 shows an observed powder X-ray diffraction pattern (PXRD) for Form 2 of bis-tosylate salt of C104 (monohydrate) carried out on a Bruker AXS D8 Endeavor diffractometer equipped with a Cu Kα radiation source (wavelength of 1.5406 Å).

FIG. 3 shows an observed powder X-ray diffraction pattern (PXRD) for the Mono Tosylate Salt of C104 (as prepared by a procedure as described in Example 5) carried out on a Bruker AXS D8 Endeavor diffractometer equipped with a Cu Kα radiation source (wavelength of 1.5406 Å).

FIG. 4 shows an observed powder X-ray diffraction pattern (PXRD) for the Bis Mesylate Salt of C104 (as prepared by a procedure as described in Example 5) carried out on a Bruker AXS D8 Endeavor diffractometer equipped with a Cu Kα radiation source (wavelength of 1.5406 Å).

FIG. 5 shows an observed powder X-ray diffraction pattern (PXRD) for the Mono Mesylate Salt of C104 (as prepared by a procedure as described in Example 5) carried out on a Bruker AXS D8 Endeavor diffractometer equipped with a Cu Kα radiation source (wavelength of 1.5406 Å).

FIG. 6 shows an observed powder X-ray diffraction pattern (PXRD) for the Mono Sulfate Salt of C104 (as prepared by a procedure as described in Example 5) carried out on a Bruker AXS D8 Endeavor diffractometer equipped with a Cu Kα radiation source (wavelength of 1.5406 Å).

FIG. 7 shows an observed powder X-ray diffraction pattern (PXRD) for the Hemi Sulfate Salt of C104 (as prepared by a procedure as described in Example 5) carried out on a Bruker AXS D8 Endeavor diffractometer equipped with a Cu Kα radiation source (wavelength of 1.5406 Å).

FIG. 8 shows an observed powder X-ray diffraction pattern (PXRD) for the hemi-barium salt of C111 (as prepared by a procedure as described in Example 8) carried out on a Bruker AXS D8 Endeavor diffractometer equipped with a Cu Kα radiation source (wavelength of 1.5406 Å).

FIG. 9 shows an observed powder X-ray diffraction pattern (PXRD) for the Form 1 of Tris Salt of C111 (as prepared by a procedure as described in Example 11 or 12) carried out on a Bruker AXS D8 Endeavor diffractometer equipped with a Cu Kα radiation source (wavelength of 1.5406 Å).

FIG. 10 shows an observed ¹³C ssNMR pattern of Form 1 of Tris Salt of C111 conducted on a 4 mm magic angle spinning (MAS) probe at MAS rates of 10 kHz positioned into a Bruker Avance III HD 400 MHZ (1H frequency) NMR spectrometer.

FIG. 11 shows an observed ¹⁹F ssNMR pattern of Form 1 of Tris Salt of C111 conducted on a 3.2 mm MAS probe with a spin rate of 20 KHz positioned into a Bruker Avance III HD 400 MHZ (¹H frequency) NMR spectrometer.

FIG. 12 shows an observed ¹⁵N ssNMR pattern of Form 1 of Tris Salt of C111 conducted on a Bruker AVANCE NEO 400 MHZ NMR spectrometer equipped with a 4 mM MAS probe with a spin rate of 20 KHz.

FIG. 13 shows a representative, observed FT-Raman spectrum of Form 1 of Tris Salt of C111, using a RAM II FT-Raman module attached to a Vertex 70 spectrometer (Bruker Optik GmbH).

FIG. 14 shows an observed powder X-ray diffraction pattern (PXRD) for C110 (as prepared by a procedure as described in Example 7) carried out on a PANalytical X′pert pro with PIXcel detector (128 channels) using Cu K radiation (α1 λ=1.54060 Å; a2=1.54443 Å; β=1.39225 Å; α1:α2 ratio=0.5).

### DETAILED DESCRIPTION OF THE INVENTION

The present invention may be understood more readily by reference to the following detailed description of exemplary embodiments of the invention and the examples included therein.

It is to be understood that this invention is not limited to specific preparation methods that may of course vary. It is also to be understood that the terminology used herein is for the purpose of describing particular embodiments only and is not intended to be limiting.

Any solid form of the present invention can be substantially pure. As used herein, the term “substantially pure” with reference to a particular solid form (e.g. a crystalline form) means that the particular solid form (e.g. the crystalline form) includes less than 15%, less than 10%, less than 5%, less than 3%, or less than 1% by weight of any other physical form of tris salt of C111.

The term “substantially the same” when used to describe X-ray powder diffraction patterns is meant to include patterns in which peaks (in terms of 20) are within the deviations specified herein.

The term “substantially the same” when used to describe an ssNMR spectrum meant to include ssNMR spectra in which peaks (in terms of chemical shifts) are within the deviations specified herein.

The term “substantially the same” when used to describe an FT-Raman spectrum meant to include FT-Raman spectra in which peaks (in terms of wavenumber) are within the deviations specified herein.

The term “about” generally means within 10%, preferably within 5%, and more preferably within 1% of a given value or range. Alternatively, the term “about” means within an acceptable standard error of the mean, when considered by one skilled in the art.

The term “tris” means 1,3-dihydroxy-2-(hydroxymethyl) propan-2-amine, also known as THAM, tromethamine, or 2-amino-2-(hydroxymethyl) propane-1,3-diol.

Tris salt of C111 means a salt of C111 made using 1,3-dihydroxy-2-(hydroxymethyl) propan-2-amine and C111. The tris is associated with the carboxylic acid moiety of C111. Unless otherwise stated, when referencing the tris salt of C111, the counterion and C111 are in a stoichiometric ratio of about 1:1 (i.e. from 0.9:1.0 to 1.0:0.9, for example, from 0.95:1.00 to 1.00:0.95). Another chemical name for tris salt of C111 is 1,3-dihydroxy-2-(hydroxymethyl) propan-2-aminium 2-[(4-{6-[(4-Cyano-2-fluorobenzyl)oxy]pyridin-2-yl}piperidin-1-yl)methyl]-1-[(2S)-oxetan-2-ylmethyl]-1H-benzimidazole-6-carboxylate, which can also be represented, for example, by one of the following structures.

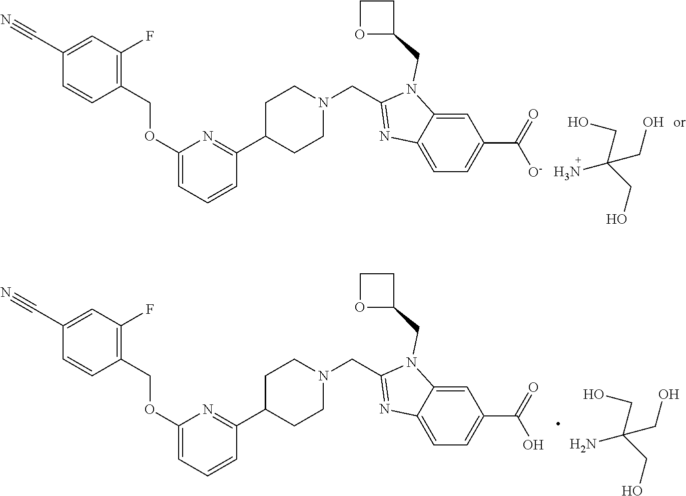 [SMILES: N#Cc1ccc(COc2cccc(C3CCN(Cc4nc5ccc(C(=O)[O-])cc5n4C[C@@H]4CCO4)CC3)n2)c(F)c1 ; NC(CO)(CO)CO ; N#Cc1ccc(COc2cccc(C3CCN(Cc4nc5ccc(C(=O)O)cc5n4C[C@@H]4CCO4)CC3)n2)c(F)c1 ; NC(CO)(CO)CO]

Those skilled in the art would readily understand that multiple nomenclatures can be used to name the same compound (including the same salt).

Every example or embodiment of solid forms of the invention may be claimed individually or grouped together in any combination with any number of each and every embodiment described herein.
Room temperature (RT) or ambient temperature: 15 to 25° C.
Dimethyl sulfoxide: DMSO.

¹H Nuclear magnetic resonance (NMR) spectra were in all cases consistent with the proposed structures. Characteristic chemical shifts (δ) are given in parts-per-million relative to the residual proton signal in the deuterated solvent (CHCl₃ at 7.27 ppm; CD₂HOD at 3.31 ppm; MeCN at 1.94 ppm; DMSO at 2.50 ppm) and are reported using conventional abbreviations for designation of major peaks: e.g. s, singlet; d, doublet; t, triplet; q, quartet; m, multiplet; br, broad. The symbol \hat{ }denotes that the ¹H NMR peak area was assumed because the peak was partially obscured by water peak. The symbol \hat{ }\hat{ }denotes that the 1H NMR peak area was assumed because the peak was partially obscured by solvent peak.

The compounds and intermediates described below were named using the naming convention provided with ACD/ChemSketch 2012, ChemDraw, File Version C10H41, Build 69045 (Advanced Chemistry Development, Inc., Toronto, Ontario, Canada). The naming convention provided with ACD/ChemSketch 2012 is well known by those skilled in the art and it is believed that the naming convention provided with ACD/ChemSketch 2012 generally comports with the IUPAC (International Union for Pure and Applied Chemistry) recommendations on Nomenclature of Organic Chemistry and the CAS Index rules. One will note that the chemical names may have only parentheses or may have parentheses and brackets. The stereochemical descriptors may also be placed at different locations within the name itself, depending on the naming convention. One of ordinary skill in the art will recognize these formatting variations and understand they provide the same chemical structure.

Pharmaceutically acceptable salts include acid addition and base salts.

Suitable acid addition salts are formed from acids which form non-toxic salts. Examples include the acetate, adipate, aspartate, benzoate, besylate, bicarbonate/carbonate, bisulfate/sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate, 1,5-naphathalenedisulfonic acid and xinafoate salts.

Suitable base salts are formed from bases which form non-toxic salts. Examples include the aluminium, arginine, benzathine, calcium, choline, diethylamine, bis(2-hydroxyethyl)amine (diolamine), glycine, lysine, magnesium, meglumine, 2-aminoethanol (olamine), potassium, sodium, 2-Amino-2-(hydroxymethyl) propane-1,3-diol (tris or tromethamine) and zinc salts.

Hemisalts of acids and bases may also be formed, for example, hemisulfate and hemicalcium salts. For a review on suitable salts, see Handbook of Pharmaceutical Salts: Properties, Selection, and Use by Stahl and Wermuth (Wiley-VCH, 2002).

Pharmaceutically acceptable salts may be prepared by one or more of three methods:
(i) by reacting a compound with the desired acid or base;
(ii) by removing an acid- or base-labile protecting group from a suitable precursor of a compound or by ring-opening a suitable cyclic precursor, for example, a lactone or lactam, using the desired acid or base; or
(iii) by converting one salt of a compound to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

Compounds and pharmaceutically acceptable salts, may exist in unsolvated and solvated forms. The term ‘solvate’ is used herein to describe a molecular complex comprising a compound or its salt, and one or more pharmaceutically acceptable solvent molecules, for example, ethanol. The term ‘hydrate’ is employed when said solvent is water. Form 1 and Form 2 described herein are believed to be unsolvated (and thus anhydrous).

A currently accepted classification system for organic hydrates is one that defines isolated site, channel, or metal-ion coordinated hydrates-see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex may have a well-defined stoichiometry independent of humidity. When, however, the solvent or water is weakly bound, as in channel solvates and hygroscopic compounds, the water/solvent content may be dependent on humidity and drying conditions. In such cases, non-stoichiometry will be the norm.

Also included within the scope of the invention are multi-component complexes (other than salts and solvates) wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallisation, by recrystallisation from solvents, or by physically grinding the components together—see Chem Commun, 17, 1889-1896, by O. Almarsson and M. J. Zaworotko (2004). For a general review of multi-component complexes, see J Pharm Sci, 64 (8), 1269-1288, by Haleblian (August 1975).

The compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterised by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterised by a phase change, typically first order (‘melting point’).

A compound may also exist in a mesomorphic state (mesophase or liquid crystal) when subjected to suitable conditions. The mesomorphic state is intermediate between the true crystalline state and the true liquid state (either melt or solution). Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N⁻N⁺ (CH₃)₃) polar head group. For more information, see Crystals and the Polarizing Microscope by N. H. Hartshorne and A. Stuart, 4^{th} Edition (Edward Arnold, 1970).

Some compounds may exhibit polymorphism and/or one or more kinds of isomerism (e.g. optical, geometric or tautomeric isomerism). The solid forms (e.g. crystalline and/or amorphous forms) of the invention may also be isotopically labelled. Such variation is implicit to C111 or its salt defined as they are by reference to their structural features and therefore within the scope of the invention.

Compounds containing one or more asymmetric carbon atoms can exist as two or more stereoisomers. Where a compound contains an alkenyl or alkenylene group, geometric cis/trans (or Z/E) isomers are possible. Where structural isomers are interconvertible via a low energy barrier, tautomeric isomerism (‘tautomerism’) can occur. This can take the form of proton tautomerism in compounds containing, for example, an imino, keto, or oxime group, or so-called valence tautomerism in compounds which contain an aromatic moiety. It follows that a single compound may exhibit more than one type of isomerism.

Certain pharmaceutically acceptable salts of C111 may also contain a counterion which is optically active (e.g. d-lactate or l-lysine) or racemic (e.g. dl-tartrate or dl-arginine).

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). Alternatively, a racemic precursor containing a chiral ester may be separated by enzymatic resolution (see, for example, Int J Mol Sci 29682-29716 by A. C. L. M. Carvaho et. al. (2015)). In the case where a compound contains an acidic or basic moiety, a salt may be formed with an optically pure base or acid such as 1-phenylethylamine or tartaric acid. The resulting diastereomeric mixture may be separated by fractional crystallization and one or both of the diastereomeric salts converted to the corresponding pure enantiomer(s) by means well known to a skilled person. Alternatively, the racemate (or a racemic precursor) may be covalently reacted with a suitable optically active compound, for example, an alcohol, amine or benzylic chloride. The resulting diastereomeric mixture may be separated by chromatography and/or fractional crystallization by means well known to a skilled person to give the separated diastereomers as single enantiomers with 2 or more chiral centers. Chiral compounds (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture. Chiral chromatography using sub- and supercritical fluids may be employed. Methods for chiral chromatography useful in some embodiments of the present invention are known in the art (see, for example, Smith, Roger M., Loughborough University, Loughborough, UK; Chromatographic Science Series (1998), 75 (SFC with Packed Columns), pp. 223-249 and references cited therein). In some relevant examples herein, columns were obtained from Chiral Technologies, Inc, West Chester, Pennsylvania, USA, a subsidiary of Daicel® Chemical Industries, Ltd., Tokyo, Japan.

When any racemate crystallises, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer. While both of the crystal forms present in a racemic mixture have identical physical properties, they may have different physical properties compared to the true racemate. Racemic mixtures may be separated by conventional techniques known to those skilled in the art-see, for example, Stereochemistry of Organic Compounds by E. L. Eliel and S. H. Wilen (Wiley, 1994).

It must be emphasised that C111 and its salts have been drawn herein in a single tautomeric form, all possible tautomeric forms are included within the scope of the invention.

The present invention includes all pharmaceutically acceptable isotopically-labeled C111 or a salt thereof wherein one or more atoms are replaced by atoms having the same atomic number, but an atomic mass or mass number different from the atomic mass or mass number which predominates in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, nitrogen, such as ¹³N and ¹⁵N, and oxygen, such as ¹⁵O, ¹⁷O and ¹⁸O.

Certain isotopically-labelled C111 or a salt thereof, for example those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds can generally be prepared by conventional techniques known to those skilled in the art or by processes analogous to those described in the accompanying Examples and Preparations using an appropriate isotopically-labeled reagent in place of the non-labeled reagent previously employed.

Pharmaceutically acceptable solvates in accordance with the invention include those wherein the solvent of crystallization may be isotopically substituted, e.g. D₂O, de-acetone, de-DMSO.

In one embodiment (Embodiment A1), the present invention provides a process for preparing bis(4-methylbenzenesulfonate) salt of 3-fluoro-4-(((6-(piperidin-4-yl) pyridin-2-yl)oxy)methyl)benzonitrile,

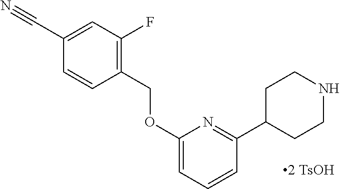 [SMILES: N#Cc1ccc(COc2cccc(C3CCNCC3)n2)c(F)c1]

which process comprising:
(a1) reacting tert-butyl 4-(6-chloropyridin-2-yl) piperidine-1-carboxylate with 3-fluoro-4-(hydroxymethyl)benzonitrile in the presence a palladium catalyst [such as palladium (II) acetate, tris(dibenzylideneacetone) dipalladium, or palladium (II) chloride], a base [such as an inorganic base, for example, potassium phosphate tribasic, cesium carbonate, potassium hydroxide, or sodium hydride], and a phosphorous ligand [e.g. a monodentate phosphorous ligand or a bidentate phosporous ligand, such as 2-dicyclohexylphosphino-2′,4′,6′-triisopropylbiphenyl (X-Phos), (2-biphenylyl)di-tert-butylphosphine (JohnPhos), 5-(di-tert-butylphosphino)-1′,3′,5′-triphenyl-1′H-[1,4′]bipyrazole (Bippyphos), 5-[bis(1-adamantyl)phosphino]-1′,3′,5′-triphenyl-1,4′-bi-1H-pyrazole (AdBippyphos), 2-(dicyclohexylphosphino) 3,6-dimethoxy-2′,4′,6′-triisopropyl-1,1′-biphenyl (BrettPhos), di(adamantan-1-yl) (2′,4′,6′-triisopropyl-3,6-dimethoxy-2-biphenyl Yl)phosphine (AdBrettPhos), 2-dicyclohexylphosphino-2′,6-bis(N,N-dimethylamino) biphenyl (CPhos), 2-dicyclohexylphosphino-2′,6′-dimethoxybiphenyl (SPhos), 2-dicyclohexylphosphino-2′-(N,N-dimethylamino) biphenyl (DavePhos), 2-di-tert-butylphosphino-2′-(N,N-dimethylamino) biphenyl (tBuDavePhos), 2-(di-tert-butylphosphino)-2′,4′,6′-triisopropyl-3,6-dimethoxy-1,1′-biphenyl (tBuBrettPhos), di-tert-butyl (2′,4′,6′-triisopropyl-3-methoxy-6-methyl-[1,1′-biphenyl]-2-yl)phosphine (RockPhos), 4,5-bis(diphenylphosphino)-9,9-dimethylxanthene (XantPhos), or bis[(2-diphenylphosphino)phenyl] ether (DPEPhos)], in a solvent system [such as one comprising anisole, acetonitrile, tetrahydrofuran, dioxane, or methyl tert-butyl ether], to form tert-butyl 4-(6-((4-cyano-2-fluorobenzyl)oxy)pyridin-2-yl) piperidine-1-carboxylate, wherein the amount of the 3-fluoro-4-(hydroxymethyl)benzonitrile is about 1.0 to about 1.1 molar equivalents to the tert-butyl 4-(6-chloropyridin-2-yl) piperidine-1-carboxylate;
(a2) adding ethanol to the reaction mixture from Step (a1);
(a3) filtering the resultant reaction mixture from Step (a2);
(b1) adding p-toluenesulfonic acid monohydrate to the filtrate from Step (a3), thereby reacting the tert-butyl 4-(6-((4-cyano-2-fluorobenzyl)oxy)pyridin-2-yl) piperidine-1-carboxylate with the p-toluenesulfonic acid monohydrate, to form a bis(4-methylbenzenesulfonate) salt of 3-fluoro-4-(((6-(piperidin-4-yl) pyridin-2-yl)oxy)methyl)benzonitrile, wherein the amount of the p-toluenesulfonic acid monohydrate is about 2.0 to about 3.0 molar equivalents to the tert-butyl 4-(6-((4-cyano-2-fluorobenzyl)oxy)pyridin-2-yl) piperidine-1-carboxylate; and
(b2) isolating the bis(4-methylbenzenesulfonate) salt of 3-fluoro-4-(((6-(piperidin-4-yl) pyridin-2-yl)oxy)methyl)benzonitrile from Step (b1).

The progress of the reaction in each of Steps (a1) and (b1) can be monitored by a variety of techniques, for example by chromatographic techniques (e.g., LC, TLC, or reverse phase HPLC). The reaction in each of Steps (a1) and (b1) is carried out for a time and under conditions sufficient to form the intended product (and to allow the reaction to go completion).

Embodiment A2 is a further embodiment of Embodiment A1, wherein the amount of the palladium catalyst [e.g., palladium (II) acetate or tris (dibenzylideneacetone) dipalladium] is about 0.1 molar % to about 2.0 molar % (e.g., about 0.15 molar % to about 0.5 molar %, or 0.25 molar %) of the tert-butyl 4-(6-chloropyridin-2-yl) piperidine-1-carboxylate in Step (a1). In some further embodiments, the palladium catalyst is palladium (II) acetate. In some yet further embodiments, the Palladium (II) acetate is dissolved in anisole to form a solution before being added to the reaction mixture in Step (a1).

Embodiment A3 is a further embodiment of Embodiment A1 or A2, wherein the amount of the phosphorous ligand (e.g. JohnPhos) is about 2.0 molar equivalent to the palladium catalyst [e.g., palladium (II) acetate] in Step (a1). In some further embodiments, the ligand is JohnPhos. In some yet further embodiments, the JohnPhos is dissolved in anisole to form a solution before being added to the reaction mixture in Step (a1).

Embodiment A4 is a further embodiment of any one of Embodiments A1 to A3, wherein the amount of the base (e.g. potassium phosphate tribasic) is about 1 to about 2 molar equivalents to the tert-butyl 4-(6-chloropyridin-2-yl) piperidine-1-carboxylate in Step (a1). In some further embodiments, the base is potassium phosphate tribasic. In some yet further embodiments, the amount of the potassium phosphate tribasic is about 1.5 to about 1.9 (e.g. 1.7) molar equivalents to the tert-butyl 4-(6-chloropyridin-2-yl) piperidine-1-carboxylate in Step (a1).

Embodiment A5 is a further embodiment of any one of Embodiments A1 to A4, wherein the volume amount of the anisole is about 5 ml/g to about 10 ml/g (e.g. about 7 ml/g to about 9 ml/g, or about 8 ml/g) based on the weight of the tert-butyl 4-(6-chloropyridin-2-yl) piperidine-1-carboxylate in Step (a1).

Embodiment A6 is a further embodiment of any one of Embodiments A1 to A5, wherein the reaction mixture is stirred at about 80 to about 120° C. (e.g. about 90 to about 110° C., or about 100° C.) for a for a time sufficient to form tert-butyl 4-(6-((4-cyano-2-fluorobenzyl)oxy)pyridin-2-yl) piperidine-1-carboxylate in Step (a1). The progress of the reaction can be monitored by a variety of techniques, for example by chromatographic techniques (e.g., LC, TLC, or reverse phase HPLC). In some embodiments, the reaction takes about 16 to about 24 hours to complete.

Embodiment A7 is a further embodiment of Embodiment A6, wherein the reaction mixture is cooled down to the ambient temperature after reaction completion in Step (a1) and before Step (a2) is carried out.

Embodiment A8 is a further embodiment of any one of Embodiments A1 to A7, wherein the volume amount of the ethanol in Step (a2) is about 0.2 to about 0.3 (e.g. 0.25) equivalent of the volume amount of the anisole in Step (a1).

Embodiment A9 is a further embodiment of any one of Embodiments A1 to A8, wherein the filtering in Step (a3) further comprises washing with ethyl acetate. In some further embodiment, the volume amount of the ethyl acetate used to wash is about 0.4 to about 0.6 (e.g. about 0.5) equivalent of the volume amount of the anisole in Step (a1).

The filtrate from Step (a3) is used in Step (b1) directly.

Embodiment A10 is a further embodiment of any one of Embodiments A1 to A9, wherein the amount of the p-toluenesulfonic acid monohydrate is about 2.0 to about 2.5 molar equivalents to the tert-butyl 4-(6-((4-cyano-2-fluorobenzyl)oxy)pyridin-2-yl) piperidine-1-carboxylate in Step (b1).

Embodiment A11 is a further embodiment of any one of Embodiments A1 to A10, wherein the amount of the p-toluenesulfonic acid monohydrate is about 2.1 to about 2.4 (e.g. 2.2) molar equivalents to the tert-butyl 4-(6-((4-cyano-2-fluorobenzyl)oxy)pyridin-2-yl) piperidine-1-carboxylate in Step (b1).

Embodiment A12 is a further embodiment of any one of Embodiments A1 to A11, wherein reaction mixture in Step (b1) is stirred at about 30° C. to about 60° C. (e.g., about 35° C. to about 50° C., or about 40° C.) for a for a time sufficient to form bis(4-methylbenzenesulfonate) salt of 3-fluoro-4-(((6-(piperidin-4-yl) pyridin-2-yl)oxy)methyl)benzonitrile. The progress of the reaction can be monitored by a variety of techniques, for example by chromatographic techniques (e.g., LC, TLC, or reverse phase HPLC).

Embodiment A13 is a further embodiment of any one of Embodiments A1 to A12, wherein the p-toluenesulfonic acid monohydrate, as a neat reagent (i.e., without premixing with a solvent), is added to reaction mixture in Step (b1).

Embodiment A14 is a further embodiment of any one of Embodiments A1 to A13, wherein isolating the bis(4-methylbenzenesulfonate) salt of 3-fluoro-4-(((6-(piperidin-4-yl) pyridin-2-yl)oxy)methyl)benzonitrile in Step (b2) comprises cooling the reaction mixture and filtering the mixture after the reaction is complete from Step (b1). In some further embodiments, cooling the reaction mixture from Step (b1) comprises cooling the reaction mixture to about 0° C. In some yet further embodiments, cooling the reaction mixture comprises cooling the reaction mixture to about 0° C. for a period of at least one hour with stirring.

Embodiment A15 is a further embodiment of Embodiment A14, wherein filtering the mixture further comprises washing the solid obtained by the filtration with anisole. In some further embodiments, filtering the mixture further comprises washing the solid obtained by the filtration with anisole and ethyl acetate.

Embodiment A16 is a further embodiment of any one of Embodiments A1 to A15, wherein the isolated bis(4-methylbenzenesulfonate) salt of 3-fluoro-4-(((6-(piperidin-4-yl) pyridin-2-yl)oxy)methyl)benzonitrile in Step (b2) is further dried, optionally under vacuum. In some further embodiments, the vacuum drying is carried out at a temperature of no more than about 30° C., at a temperature of no more than about 35° C., at a temperature of no more than about 40° C., at a temperature of no more than about 45° C., at a temperature of no more than about 50° C., or at a temperature of no more than about 60° C. In some yet further embodiments, the vacuum drying is carried out at a temperature of no more than about 40° C.

Embodiment A17 is a further embodiment of any one of Embodiments A1 to A16, wherein the bis(4-methylbenzenesulfonate) salt of 3-fluoro-4-(((6-(piperidin-4-yl) pyridin-2-yl)oxy)methyl)benzonitrile isolated in Step (b2) is an anhydrous form.

In one embodiment (Embodiment B1), the present invention provides a process for preparing bis(4-methylbenzenesulfonate) salt of 3-fluoro-4-(((6-(piperidin-4-yl) pyridin-2-yl)oxy)methyl)benzonitrile,

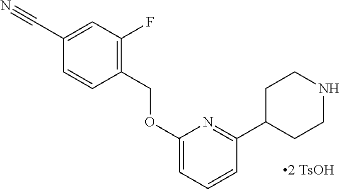 [SMILES: N#Cc1ccc(COc2cccc(C3CCNCC3)n2)c(F)c1]

which process comprising:
(a1) reacting tert-butyl 4-(6-chloropyridin-2-yl) piperidine-1-carboxylate with 3-fluoro-4-(hydroxymethyl)benzonitrile in the presence a palladium catalyst [such as palladium (II) acetate, tris (dibenzylideneacetone) dipalladium, or palladium (II) chloride], a base [such as an inorganic base for example potassium phosphate tribasic, cesium carbonate, potassium hydroxide, or sodium Hydride], and a phosphorous ligand [such as a monodentate phosphorous ligand or a bidentate phosphorous ligand, for example, 2 dicyclohexylphosphino-2′,4′,6′-triisopropylbiphenyl (X Phos), (2 bBiphenylyl)di tert butylphosphine (JohnPhos), 5-(di-tert butylphosphino) 1′,3′,5′ triphenyl-1′H-[1,4′]bipyrazole (Bippyphos), 5-[bis (1 adamantyl)phosphino]-1′,3′,5′-triphenyl-1,4′-bi-1H-pyrazole (AdBippyphos), 2-(dicyclohexylphosphino)-3,6-dimethoxy-2′,4′,6′-triisopropyl-1,1′-biphenyl (BrettPhos), di(adamantan-1-yl) (2′,4′,6′-triisopropyl-3,6-dimethoxy-2-biphenyl (CPhos), 2-dicyclohexylphosphino-2′,6′-dimethoxybiphenyl (SPhos), 2-dicyclohexylphosphino-2′-(N,N-dimethylamino) biphenyl (DavePhos), 2-di-tert-butylphosphino-2′-(N,N-dimethylamino) biphenyl (tBuDavePhos), 2-(di-tert-butylphosphino)-2′,4′,6′-triisopropyl-3,6-dimethoxy-1,1′-biphenyl (tBuBrettPhos), di-tert-butyl (2′,4′,6′-triisopropyl-3-methoxy-6-methyl-[1,1′-biphenyl]-2-yl)phosphine (RockPhos), 4,5-bis(diphenylphosphino)-9,9-dimethylxanthene (XantPhos), or bis[(2-diphenylphosphino)phenyl] ether (DPEPhos)], in a solvent system [such as one comprising anisole, acetonitrile, tetrahydrofuran, dioxane, or methyl tert-butyl ether], to form the tert-butyl 4-(6-((4-cyano-2-fluorobenzyl)oxy)pyridin-2-yl) piperidine-1-carboxylate, wherein the amount of the 3-fluoro-4-(hydroxymethyl)benzonitrile is about 1.0 to about 1.1 molar equivalents to the tert-butyl 4-(6-chloropyridin-2-yl) piperidine-1-carboxylate;
(a2) adding water, ethyl acetate, and ethanol to the reaction mixture from Step (a1);
(a3) separating the organic phase from Step (a2) from the aqueous phase;
(b1) adding p-toluenesulfonic acid monohydrate to the separated organic phase from Step (a3), thereby reacting the tert-butyl 4-(6-((4-cyano-2-fluorobenzyl)oxy)pyridin-2-yl) piperidine-1-carboxylate with the p-toluenesulfonic acid monohydrate, to form bis(4-methylbenzenesulfonate) salt of 3-fluoro-4-(((6-(piperidin-4-yl) pyridin-2-yl)oxy)methyl)benzonitrile, wherein the amount of the p-toluenesulfonic acid monohydrate is about 2.0 to about 3.0 molar equivalents to the tert-butyl 4-(6-((4-cyano-2-fluorobenzyl)oxy)pyridin-2-yl) piperidine-1-carboxylate; and
(b2) isolating the bis(4-methylbenzenesulfonate) salt of 3-fluoro-4-(((6-(piperidin-4-yl) pyridin-2-yl)oxy)methyl)benzonitrile from Step (b1).

The progress of the reaction in each of Steps (a1) and (b1) can be monitored by a variety of techniques, for example by chromatographic techniques (e.g., LC, TLC, or reverse phase HPLC). The reaction in each of Steps (a1) and (b1) is carried out for a time and under conditions sufficient to form the intended product (and to allow the reaction to go completion).

Embodiment B2 is a further embodiment of Embodiment B1, wherein the amount of the palladium catalyst [e.g., palladium (II) acetate] is about 0.1 molar % to about 0.75 molar % (e.g. 0.25 molar %) of the tert-butyl 4-(6-chloropyridin-2-yl) piperidine-1-carboxylate in Step (a1). In some further embodiments, the palladium catalyst is palladium (II) acetate. In some yet further embodiments, the palladium (II) acetate is dissolved in anisole to form a solution before being added to the reaction mixture in Step (a1).

Embodiment B3 is a further embodiment of Embodiment B1 or B2, wherein the amount of the ligand (e.g. X-Phos) is about 1.0 molar equivalent to the palladium catalyst [e.g., palladium (II) acetate] in Step (a1). In some further embodiments, the ligand is X-Phos. In some yet further embodiments, the X-Phos is dissolved in anisole to form a solution before being added to the reaction mixture in Step (a1).

Embodiment B4 is a further embodiment of any one of Embodiments B1 to B3, wherein the amount of the base (e.g. potassium phosphate tribasic) is about 1 to about 2 molar equivalents to the tert-butyl 4-(6-chloropyridin-2-yl) piperidine-1-carboxylate in Step (a1). In some further embodiments, the base is potassium phosphate tribasic. In some yet further embodiments, the amount of the potassium phosphate tribasic is about 1.5 to about 1.9 (e.g. 1.7) molar equivalents to the tert-butyl 4-(6-chloropyridin-2-yl) piperidine-1-carboxylate in Step (a1).

Embodiment B5 is a further embodiment of any one of Embodiments B1 to B4, wherein the volume amount of the anisole is about 5 ml/g to about 10 ml/g (e.g. about 8 ml/g) based on the weight of the tert-butyl 4-(6-chloropyridin-2-yl) piperidine-1-carboxylate in Step (a1).

Embodiment B6 is a further embodiment of any one of Embodiments B1 to B5, wherein the reaction mixture is stirred at about 100° C. for a for a time sufficient to form tert-butyl 4-(6-((4-cyano-2-fluorobenzyl)oxy)pyridin-2-yl) piperidine-1-carboxylate in Step (a1). The progress of the reaction can be monitored by a variety of techniques, for example by chromatographic techniques (e.g., LC, TLC, or reverse phase HPLC). In some embodiments, the reaction takes about 16-24 hours to complete.

Embodiment B7 is a further embodiment of Embodiment B6, wherein the reaction mixture is cooled down to ambient temperature after reaction completion in Step (a1) and before Step (a2) is carried out.

Embodiment B8 is a further embodiment of any one of Embodiments B1 to B7, wherein the volume amount of the water in Step (a2) is about 0.3 to about 0.45 (e.g. 0.375) equivalent of the volume amount of the anisole in Step (a1).

Embodiment B9 is a further embodiment of any one of Embodiments B1 to B8, wherein the volume amount of the ethanol in Step (a2) is about 0.2 to about 0.3 (e.g. 0.25) equivalent of the volume amount of the anisole in Step (a1).

Embodiment B10 is a further embodiment of any one of Embodiments B1 to B8, wherein the volume amount of the ethyl acetate in Step (a2) is about 0.40 to about 0.60 (e.g. 0.50) equivalent of the volume amount of the anisole in Step (a1).

After the water, ethyl acetate, and ethanol was added in Step (a2), the resulting mixture is sufficiently mixed before the two layers are allowed to settle and then the organic layer (or the organic phase) is separated from the aqueous phase in Step (a3). The separated organic layer (or the organic phase) from Step (a3) is used in Step (b1) directly.

Embodiment B11 is a further embodiment of any one of Embodiments B1 to B10, wherein the amount of the p-toluenesulfonic acid monohydrate is about 2.1 to about 2.5 molar equivalents to the tert-butyl 4-(6-((4-cyano-2-fluorobenzyl)oxy)pyridin-2-yl) piperidine-1-carboxylate in Step (b1).

Embodiment B12 is a further embodiment of any one of Embodiments B1 to B11, wherein the amount of the p-toluenesulfonic acid monohydrate is about 2.1 to about 2.4 (e.g. 2.2) molar equivalents to the tert-butyl 4-(6-((4-cyano-2-fluorobenzyl)oxy)pyridin-2-yl) piperidine-1-carboxylate in Step (b1).

Embodiment B13 is a further embodiment of any one of Embodiments B1 to B12, wherein reaction mixture in Step (b1) is stirred at about 40° C. for a for a time sufficient to form bis(4-methylbenzenesulfonate) salt of 3-fluoro-4-(((6-(piperidin-4-yl) pyridin-2-yl)oxy)methyl)benzonitrile. The progress of the reaction can be monitored by a variety of techniques, for example by chromatographic techniques (e.g., LC, TLC, or reverse phase HPLC).

Embodiment B14 is a further embodiment of any one of Embodiments B1 to B13, wherein the p-toluenesulfonic acid monohydrate, as a neat reagent (i.e., without premixing with a solvent), is added to reaction mixture in Step (b1).

Embodiment B15 is a further embodiment of any one of Embodiments B1 to B14, wherein isolating the bis(4-methylbenzenesulfonate) salt of 3-fluoro-4-(((6-(piperidin-4-yl) pyridin-2-yl)oxy)methyl)benzonitrile in Step (b2) comprises cooling the reaction mixture and filtering the mixture after the reaction is complete from Step (b1). In some further embodiments, cooling the reaction mixture from Step (b1) comprises cooling the reaction mixture to about 5° C. In some yet further embodiments, cooling the reaction mixture comprises cooling the reaction mixture to about 5° C. for a period of at least one hour with stirring.

Embodiment B16 is a further embodiment of Embodiment B15, wherein filtering the mixture further comprises washing the solid obtained by the filtration with ethyl acetate.

Embodiment B17 is a further embodiment of any one of Embodiments B1 to B16, wherein the isolated bis(4-methylbenzenesulfonate) salt of 3-fluoro-4-(((6-(piperidin-4-yl) pyridin-2-yl)oxy)methyl)benzonitrile in Step (b2) is further dried, optionally under vacuum. In some further embodiments, the vacuum drying is carried out at a temperature of no more than about 30° C., at a temperature of no more than about 35° C., at a temperature of no more than about 40° C., at a temperature of no more than about 45° C., at a temperature of no more than about 50° C., or at a temperature of no more than about 60° C. In some yet further embodiments, the vacuum drying is carried out at a temperature of no more than about 40° C.

Embodiment B18 is a further embodiment of any one of Embodiments B1 to B17, wherein the bis(4-methylbenzenesulfonate) salt of 3-fluoro-4-(((6-(piperidin-4-yl) pyridin-2-yl)oxy)methyl)benzonitrile isolated in Step (b2) is a monohydrate of bis(4-methylbenzenesulfonate) salt of 3-fluoro-4-(((6-(piperidin-4-yl) pyridin-2-yl)oxy)methyl)benzonitrile.

In one embodiment (Embodiment C1), the present invention provides a process for preparing bis(4-methylbenzenesulfonate) salt of 3-fluoro-4-(((6-(piperidin-4-yl) pyridin-2-yl)oxy)methyl)benzonitrile,

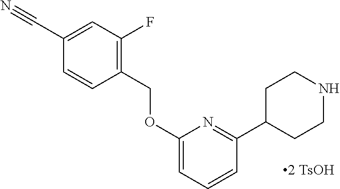 [SMILES: N#Cc1ccc(COc2cccc(C3CCNCC3)n2)c(F)c1]

which process comprising:
(a1) reacting tert-butyl 4-(6-chloropyridin-2-yl) piperidine-1-carboxylate with 3-fluoro-4-(hydroxymethyl)benzonitrile in the presence of a copper catalyst [such as copper (I) iodide, copper (I) acetate, tetrakisacetonitrile copper (I) triflate, tetrakisacetonitrile copper (I) hexafluorophosphate, or copper trifluoromethanesulfonate], a base [such as sodium tert-pentoxide, cesium carbonate, potassium phosphate, potassium hexamethylenedisilazide, or sodium tert-butoxide], and a ligand [such as N,N′-bis(1-naphthylmethyl) oxamide or N,N′-diphenylethyloxalamide], in a solvent system [such as one comprising 1,4-dioxane, anisole, methyl tert-butyl ether (MTBE), or 2-methyl-tetrahydrofuran (2-MeTHF)], to form tert-butyl 4-(6-((4-cyano-2-fluorobenzyl)oxy)pyridin-2-yl) piperidine-1-carboxylate, wherein the amount of the 3-fluoro-4-(hydroxymethyl)benzonitrile is about 1.1 to about 1.3 (e.g. 1.2) molar equivalents to the tert-butyl 4-(6-chloropyridin-2-yl) piperidine-1-carboxylate;
(a2) filtering the reaction mixture from Step (a1), wherein the filtering further comprises washing with methyl tert-butyl ether (MTBE);
(a3) concentrating the filtrate from Step (2) to obtain tert-butyl 4-(6-((4-cyano-2-fluorobenzyl)oxy)pyridin-2-yl) piperidine-1-carboxylate;
(b1) dissolving the tert-butyl 4-(6-((4-cyano-2-fluorobenzyl)oxy)pyridin-2-yl) piperidine-1-carboxylate from Step (a3) in a solvent system comprising 1,4-dioxane and MTBE to form a solution; and then adding p-toluenesulfonic acid monohydrate to the solution, thereby reacting the tert-butyl 4-(6-((4-cyano-2-fluorobenzyl)oxy)pyridin-2-yl) piperidine-1-carboxylate with the p-toluenesulfonic acid monohydrate, to form bis(4-methylbenzenesulfonate) salt of 3-fluoro-4-(((6-(piperidin-4-yl) pyridin-2-yl)oxy)methyl)benzonitrile, wherein the amount of the p-toluenesulfonic acid monohydrate is about 2.0 to about 2.5 (e.g. about 2.1) molar equivalents to the tert-butyl 4-(6-((4-cyano-2-fluorobenzyl)oxy)pyridin-2-yl) piperidine-1-carboxylate; and
(b2) isolating the bis(4-methylbenzenesulfonate) salt of 3-fluoro-4-(((6-(piperidin-4-yl) pyridin-2-yl)oxy)methyl)benzonitrile from Step (b1).

The progress of the reaction in each of Steps (a1) and (b1) can be monitored by a variety of techniques, for example by chromatographic techniques (e.g., 1HNMR, TLC, or reverse phase HPLC). The reaction in each of Steps (a1) and (b1) is carried out for a time and under conditions sufficient to form the intended product (and to allow the reaction to go completion).

Embodiment C2 is a further embodiment of Embodiment C1, wherein the amount of the copper catalyst [such as tetrakisacetonitrile copper (I) triflate, copper (I) iodide, copper (I) acetate, tetrakisacetonitrile copper (I) hexafluorophosphate, or copper trifluoromethanesulfonate] is about 4.0 molar % to about 20 molar % (e.g. about 4.0 molar % to about 6.0 molar %, or about 5.0 molar %) of the tert-butyl 4-(6-chloropyridin-2-yl) piperidine-1-carboxylate in Step (a1). In some further embodiments, the copper catalyst is tetrakisacetonitrile copper (I) triflate.

Embodiment C3 is a further embodiment of Embodiment C1 or C2, wherein the amount of the ligand (e.g. N,N′-bis(1-naphthylmethyl) oxamide) is about 1.0 molar equivalent to the copper catalyst [such as tetrakisacetonitrile copper (I) triflate] in Step (a1). In some further embodiments, the ligand is N,N′-bis(1-naphthylmethyl) oxamide.

Embodiment C4 is a further embodiment of any one of Embodiments C1 to C3, wherein the amount of the base (e.g. sodium tert-pentoxide) is about 1 to about 2 molar equivalents to the tert-butyl 4-(6-chloropyridin-2-yl) piperidine-1-carboxylate in Step (a1). In some further embodiments, the base is sodium tert-pentoxide. In some yet further embodiments, the amount of the sodium tert-pentoxide is about 1.3 to about 1.7 (e.g. 1.5) molar equivalents to the tert-butyl 4-(6-chloropyridin-2-yl) piperidine-1-carboxylate in Step (a1). In some still further embodiments, the sodium tert-pentoxide is dissolved in toluene to form a solution before being added to the reaction mixture in Step (a1).

Embodiment C5 is a further embodiment of any one of Embodiments C1 to C4, wherein the solvent system in Step (a1) is 1,4-dioxane and the volume amount of the 1,4-dioxane is about 7 ml/g to about 9 ml/g (e.g. about 8 ml/g) based on the weight of the tert-butyl 4-(6-chloropyridin-2-yl) piperidine-1-carboxylate in Step (a1).

Embodiment C6 is a further embodiment of any one of Embodiments C1 to C5, wherein the reaction mixture is stirred at about 80° C. for a for a time sufficient to form tert-butyl 4-(6-((4-cyano-2-fluorobenzyl)oxy)pyridin-2-yl) piperidine-1-carboxylate in Step (a1). The progress of the reaction can be monitored by a variety of techniques, for example by chromatographic techniques (e.g., LC, TLC, or reverse phase HPLC). In some embodiments, the reaction takes about 16-24 hours to complete.

Embodiment C7 is a further embodiment of Embodiment C6, wherein the reaction mixture is cooled down to the ambient temperature after reaction completion in Step (a1) and before Step (a2) is carried out.

Embodiment C8 is a further embodiment of any one of Embodiments C1 to C7, wherein the volume amount of the MTBE in Step (a2) is about 0.2 to about 0.3 (e.g. 0.25) equivalent of the volume amount of the solvent 1,4-dioxane in Step (a1). In some further embodiments, the MTBE is divided in two portions for the wash. In some yet further embodiments, reaction vessel is further rinsed with 1,4-dioxane [e.g. about 0.2 volume equivalent of the volume amount of the solvent 1,4-dioxane in Step (a1)], which is also filtered, before the MTBE wash (or washes).

In Step (a3), the solvents are removed and the tert-butyl 4-(6-((4-cyano-2-fluorobenzyl)oxy)pyridin-2-yl) piperidine-1-carboxylate obtained is carried over to Step (b1).

Embodiment C9 is a further embodiment of any one of Embodiments C1 to C8, wherein the volume amount of the 1,4-dioxane used to dissolve the tert-butyl 4-(6-((4-cyano-2-fluorobenzyl)oxy)pyridin-2-yl) piperidine-1-carboxylate in Step (b1) is about 1.5 to about 1.9 (e.g. about 1.67) ml/g based on the weight of the tert-butyl 4-(6-chloropyridin-2-yl) piperidine-1-carboxylate used in Step (a1).

Embodiment C10 is a further embodiment of any one of Embodiments C1 to C9, wherein the volume amount of the MTBE used to dissolve the tert-butyl 4-(6-((4-cyano-2-fluorobenzyl)oxy)pyridin-2-yl) piperidine-1-carboxylate in Step (b1) is about 0.75 to about 0.95 (e.g. about 0.83) ml/g based on the weight of the tert-butyl 4-(6-chloropyridin-2-yl) piperidine-1-carboxylate used in Step (a1).

Embodiment C11 is a further embodiment of any one of Embodiments C1 to C10, wherein the amount of the p-toluenesulfonic acid monohydrate is about 2.0 to about 2.2 (e.g. about 2.1) molar equivalents to the tert-butyl 4-(6-((4-cyano-2-fluorobenzyl)oxy)pyridin-2-yl) piperidine-1-carboxylate in Step (b1), assuming 100% reaction yield from Step (a1).

Embodiment C12 is a further embodiment of any one of Embodiments C1 to C11, wherein the p-toluenesulfonic acid monohydrate, as a neat reagent (i.e., without premixing with a solvent), is added to reaction mixture in Step (b1).

Embodiment C13 is a further embodiment of any one of Embodiments C1 to C12, wherein reaction mixture in Step (b1) is stirred at about 40° C. for a for a time sufficient to form bis(4-methylbenzenesulfonate) salt of 3-fluoro-4-(((6-(piperidin-4-yl) pyridin-2-yl)oxy)methyl)benzonitrile. The progress of the reaction can be monitored by a variety of techniques, for example by chromatographic techniques (e.g., LC, TLC, or reverse phase HPLC). In some further embodiments, additional 1,4-dioxane is added to mobilize the reaction slurry. The volume amount of the additional 1,4-dioxane is about 3.0 to about 3.8 (e.g. about 3.3) ml/g based on the weight of the tert-butyl 4-(6-chloropyridin-2-yl) piperidine-1-carboxylate used in Step (a1).

Embodiment C14 is a further embodiment of any one of Embodiments C1 to C13, wherein isolating the bis(4-methylbenzenesulfonate) salt of 3-fluoro-4-(((6-(piperidin-4-yl) pyridin-2-yl)oxy)methyl)benzonitrile in Step (b2) comprises cooling the reaction mixture and filtering the mixture after the reaction is complete from Step (b1). In some further embodiments, cooling the reaction mixture from Step (b1) comprises cooling the reaction mixture to about 0-5° C. In some yet further embodiments, cooling the reaction mixture comprises cooling the reaction mixture to about 0° C. for a period of at least about one hour with stirring.

Embodiment C15 is a further embodiment of Embodiment C14, wherein filtering the mixture further comprises washing the solid obtained by the filtration with a mixture of 1,4-dioxane:MTBE (1:1 volume ratio). The amount of the 1,4-dioxane:MTBE wash is about 3.0 to about 3.8 (e.g. about 3.3) ml/g based on the weight of the tert-butyl 4-(6-chloropyridin-2-yl) piperidine-1-carboxylate used in Step (a1).

Embodiment C16 is a further embodiment of any one of Embodiments C1 to C15, wherein the isolated bis(4-methylbenzenesulfonate) salt of 3-fluoro-4-(((6-(piperidin-4-yl) pyridin-2-yl)oxy)methyl)benzonitrile in Step (b2) is further dried, optionally under vacuum. In some further embodiments, the vacuum drying is carried out at a temperature of no more than about 30° C., at a temperature of no more than about 35° C., at a temperature of no more than about 40° C., at a temperature of no more than about 45° C., at a temperature of no more than about 50° C., or at a temperature of no more than about 60° C. In some yet further embodiments, the vacuum drying is carried out at a temperature of no more than about 40° C.

Embodiment C17 is a further embodiment of any one of Embodiments C1 to C16, wherein the bis(4-methylbenzenesulfonate) salt of 3-fluoro-4-(((6-(piperidin-4-yl) pyridin-2-yl)oxy)methyl)benzonitrile isolated in Step (b2) is an anhydrous form of bis(4-methylbenzenesulfonate) salt of 3-fluoro-4-(((6-(piperidin-4-yl) pyridin-2-yl)oxy)methyl)benzonitrile.

In one embodiment (Embodiment D1), the present invention provides an intermediate useful for preparing C111 or tris salt of C111, which is:
anhydrous 3-fluoro-4-(((6-(piperidin-4-yl) pyridin-2-yl)oxy)methyl)benzonitrile bis(4-methylbenzenesulfonate) salt;
monohydrate of 3-fluoro-4-(((6-(piperidin-4-yl) pyridin-2-yl)oxy)methyl)benzonitrile bis(4-methylbenzenesulfonate) salt;
mono tosylate salt of 3-fluoro-4-(((6-(piperidin-4-yl) pyridin-2-yl)oxy)methyl)benzonitrile;
bis mesylate salt of 3-fluoro-4-(((6-(piperidin-4-yl) pyridin-2-yl)oxy)methyl)benzonitrile;
mono mesylate salt of 3-fluoro-4-(((6-(piperidin-4-yl) pyridin-2-yl)oxy)methyl)benzonitrile;
mono sulfate salt of 3-fluoro-4-(((6-(piperidin-4-yl) pyridin-2-yl)oxy)methyl)benzonitrile;
hemi sulfate salt of 3-fluoro-4-(((6-(piperidin-4-yl) pyridin-2-yl)oxy)methyl)benzonitrile;
crystalline methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate; or
hemi-barium salt of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid.

Embodiment D2 is a further embodiment of Embodiment D1, wherein the present invention provides an anhydrous crystalline form of 3-fluoro-4-(((6-(piperidin-4-yl) pyridin-2-yl)oxy)methyl)benzonitrile bis(4-methylbenzenesulfonate) salt.

Embodiment D2A is a further embodiment of Embodiment D2, wherein the anhydrous crystalline form is Form 1 of anhydrous 3-fluoro-4-(((6-(piperidin-4-yl) pyridin-2-yl)oxy)methyl)benzonitrile bis(4-methylbenzenesulfonate) salt, and wherein Form 1 has a powder X-ray diffraction pattern (PXRD) comprising at least two peaks, in terms of 2θ, selected from those at 13.3±0.2°, 15.5±0.2°, and 17.2±0.2°.

Embodiment D2A1 is a further embodiment of Embodiment D2A, wherein Form 1 has a PXRD comprising at least three peaks, in terms of 2θ, at 13.3±0.2°, 15.5±0.2°, and 17.2±0.2°.

Embodiment D2A2 is a further embodiment of Embodiment D2A, wherein Form 1 has a PXRD comprising at least two peaks, in terms of 2θ, selected from those at 12.8±0.2°, 13.3±0.2°, 14.7±0.2°, 15.5±0.2°, and 17.2±0.2°.

Embodiment D2A3 is a further embodiment of Embodiment D2A, wherein Form 1 has a PXRD comprising at least three peaks, in terms of 2θ, selected from those at 12.8±0.2°, 13.3±0.2°, 14.7±0.2°, 15.5±0.2°, and 17.2±0.2°.

Embodiment D2A4 is a further embodiment of Embodiment D2A, wherein Form 1 has a PXRD comprising peaks, in terms of 2θ, at 12.8±0.2°, 13.3±0.2°, 14.7±0.2°, 15.5±0.2°, and 17.2±0.2°.

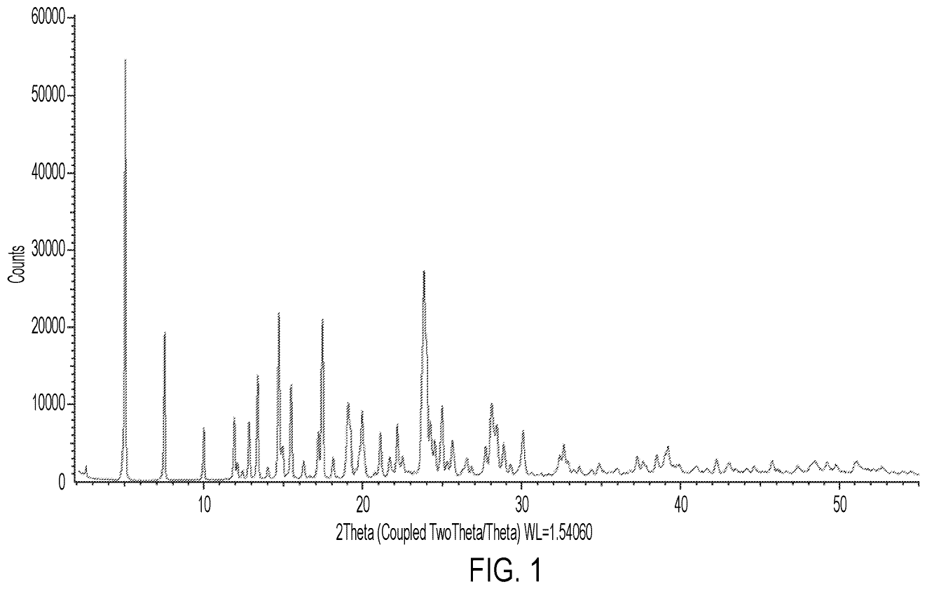

Embodiment D2A5 is a further embodiment of Embodiment D2A, wherein Form 1 has a PXRD substantially as FIG. 1.

Embodiment D3 is a further embodiment of Embodiment D1, wherein the present invention provides a monohydrate crystalline form of 3-fluoro-4-(((6-(piperidin-4-yl) pyridin-2-yl)oxy)methyl)benzonitrile bis(4-methylbenzenesulfonate) salt.

Embodiment D3A is a further embodiment of Embodiment D3, wherein the monohydrate crystalline form is Form 2 of monohydrate of 3-fluoro-4-(((6-(piperidin-4-yl) pyridin-2-yl)oxy)methyl)benzonitrile bis(4-methylbenzenesulfonate) salt, and wherein Form 2 has a powder X-ray diffraction pattern (PXRD) comprising at least two peaks, in terms of 2θ, selected from those at 13.0±0.2°, 13.7±0.2°, and 22.7±0.2°.

Embodiment D3A1 is a further embodiment of Embodiment D3A, wherein Form 2 has a PXRD comprising at least three peaks, in terms of 2θ, at 13.0±0.2°, 13.7±0.2°, and 22.7±0.2°.

Embodiment D3A2 is a further embodiment of Embodiment D3A, wherein Form 2 has a PXRD comprising at least two peaks, in terms of 2θ, selected from those at 13.0±0.2°, 13.7±0.2°, 17.0±0.2°, 22.7±0.2°, and 27.9±0.2°.

Embodiment D3A3 is a further embodiment of Embodiment D3A, wherein Form 2 has a PXRD comprising at least three peaks, in terms of 2θ, selected from those at 13.0±0.2°, 13.7±0.2°, 17.0±0.2°, 22.7±0.2°, and 27.9±0.2°.

Embodiment D3A4 is a further embodiment of Embodiment D3A, wherein Form 2 has a PXRD comprising peaks, in terms of 2θ, at 13.0±0.2°, 13.7±0.2°, 17.0±0.2°, 22.7±0.2°, and 27.9±0.2°.

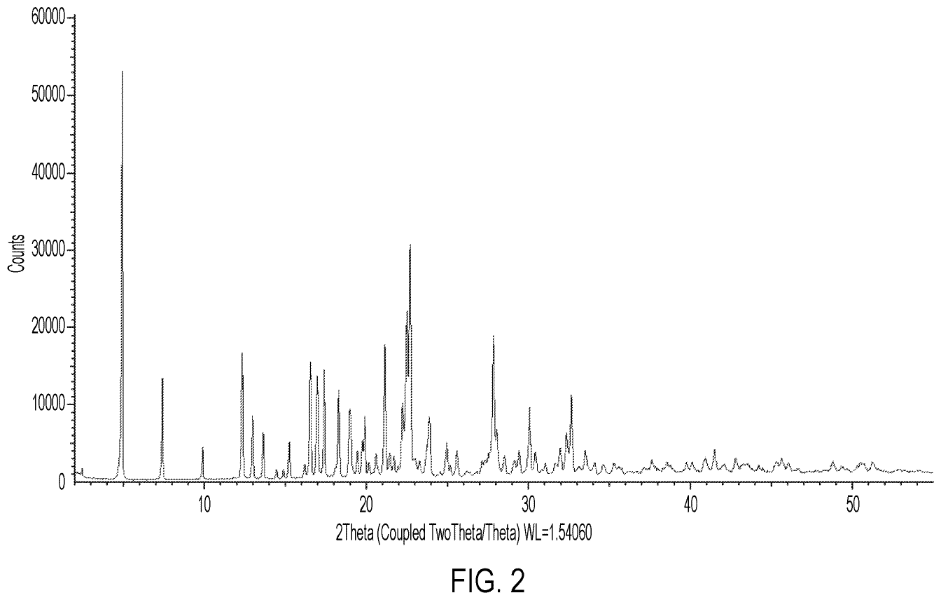

Embodiment D3A5 is a further embodiment of Embodiment D3A, wherein Form 2 has a PXRD substantially as FIG. 2.

Embodiment D4 is a further embodiment of Embodiment D1, wherein the present invention provides mono tosylate salt of 3-fluoro-4-(((6-(piperidin-4-yl) pyridin-2-yl)oxy)methyl)benzonitrile.

Embodiment D4A is a further embodiment of Embodiment D4, wherein the mono tosylate salt of 3-fluoro-4-(((6-(piperidin-4-yl) pyridin-2-yl)oxy)methyl)benzonitrile is crystalline.

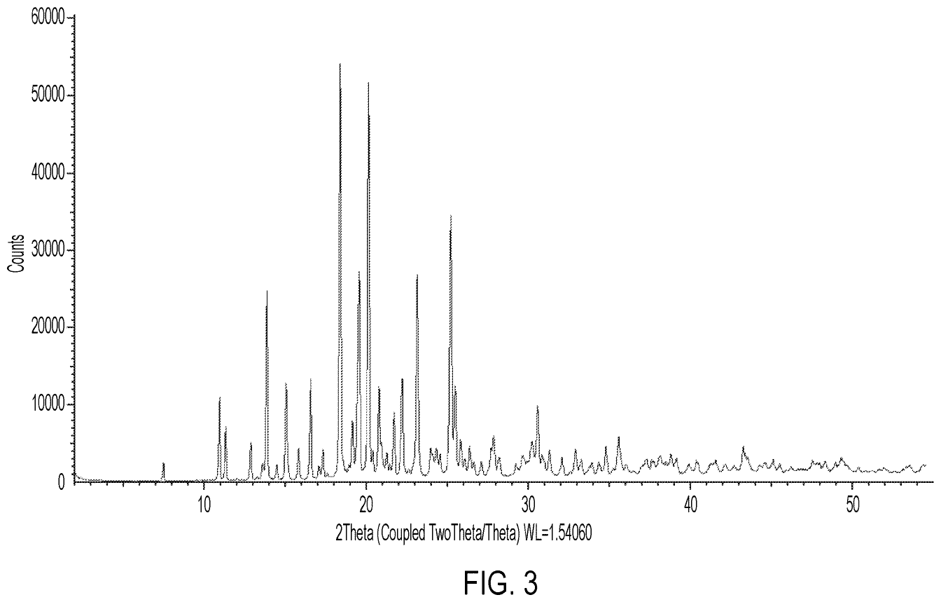

Embodiment D4A1 is a further embodiment of Embodiment D4A, wherein the crystalline mono tosylate salt of 3-fluoro-4-(((6-(piperidin-4-yl) pyridin-2-yl)oxy)methyl)benzonitrile has a PXRD substantially as FIG. 3.

Embodiment D5 is a further embodiment of Embodiment D1, wherein the present invention provides bis mesylate salt of 3-fluoro-4-(((6-(piperidin-4-yl) pyridin-2-yl)oxy)methyl)benzonitrile.

Embodiment D5A is a further embodiment of Embodiment D5, wherein the bis mesylate salt of 3-fluoro-4-(((6-(piperidin-4-yl) pyridin-2-yl)oxy)methyl)benzonitrile is crystalline.

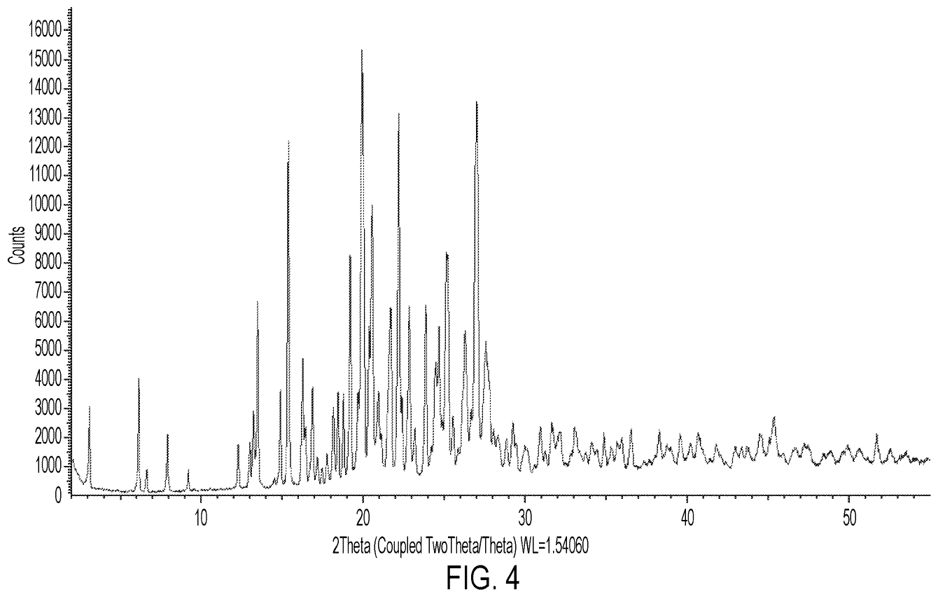

Embodiment D5A1 is a further embodiment of Embodiment D5A, wherein the crystalline bis mesylate salt of 3-fluoro-4-(((6-(piperidin-4-yl) pyridin-2-yl)oxy)methyl)benzonitrile has a PXRD substantially as FIG. 4.

Embodiment D6 is a further embodiment of Embodiment D1, wherein the present invention provides mono mesylate salt of 3-fluoro-4-(((6-(piperidin-4-yl) pyridin-2-yl)oxy)methyl)benzonitrile. Embodiment D6A is a further embodiment of Embodiment D6, wherein the mono mesylate salt of 3-fluoro-4-(((6-(piperidin-4-yl) pyridin-2-yl)oxy)methyl)benzonitrile is crystalline.

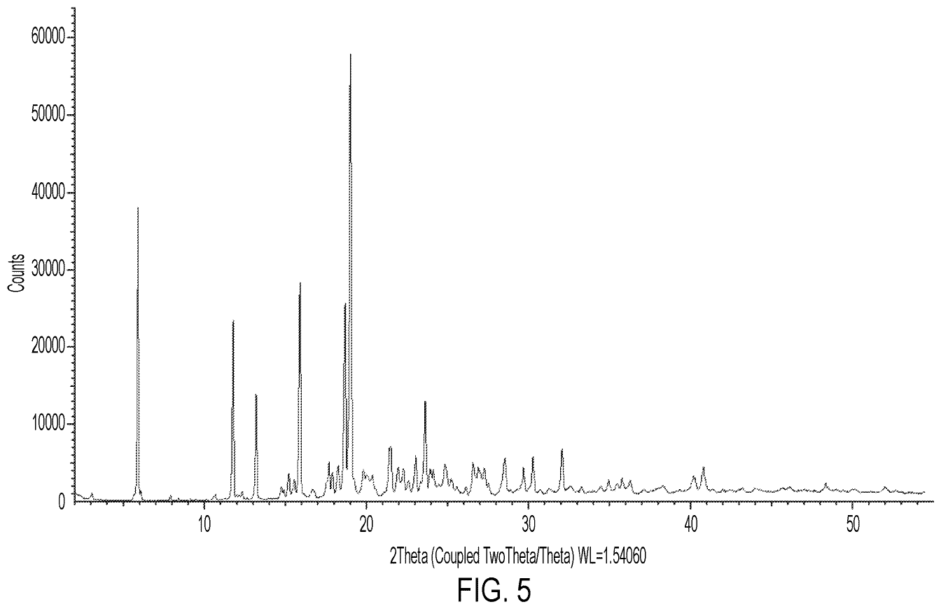

Embodiment D6A1 is a further embodiment of Embodiment D6A, wherein the crystalline mono mesylate salt of 3-fluoro-4-(((6-(piperidin-4-yl) pyridin-2-yl)oxy)methyl)benzonitrile has a PXRD substantially as FIG. 5.

Embodiment D7 is a further embodiment of Embodiment D1, wherein the present invention provides mono sulfate salt of 3-fluoro-4-(((6-(piperidin-4-yl) pyridin-2-yl)oxy)methyl)benzonitrile.

Embodiment D7A is a further embodiment of Embodiment D7, wherein the mono sulfate salt of 3-fluoro-4-(((6-(piperidin-4-yl) pyridin-2-yl)oxy)methyl)benzonitrile is crystalline.

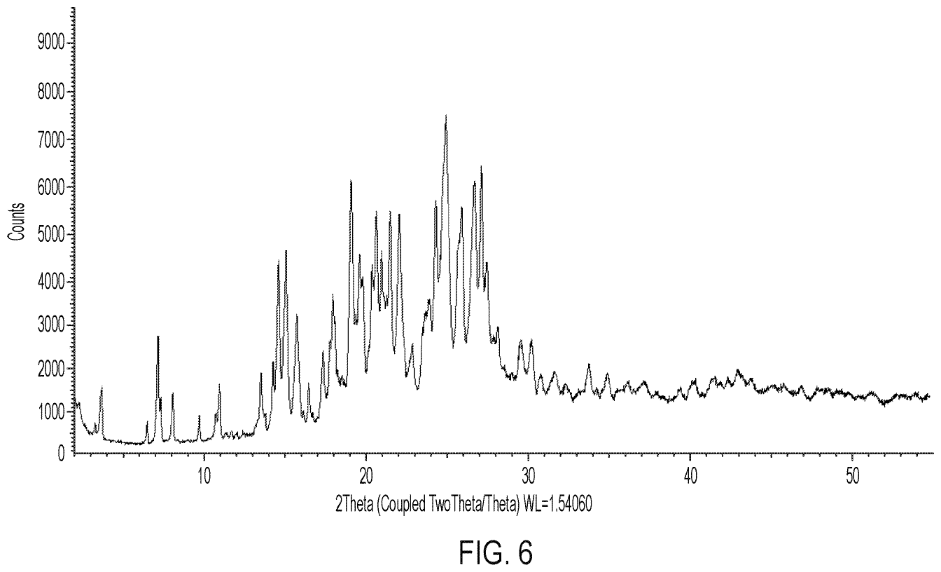

Embodiment D7A1 is a further embodiment of Embodiment D7A, wherein the crystalline mono sulfate salt of 3-fluoro-4-(((6-(piperidin-4-yl) pyridin-2-yl)oxy)methyl)benzonitrile has a PXRD substantially as FIG. 6.

Embodiment D8 is a further embodiment of Embodiment D1, wherein the present invention provides hemi sulfate salt of 3-fluoro-4-(((6-(piperidin-4-yl) pyridin-2-yl)oxy)methyl)benzonitrile.

Embodiment D8A is a further embodiment of Embodiment D8, wherein the hemi sulfate salt of 3-fluoro-4-(((6-(piperidin-4-yl) pyridin-2-yl)oxy)methyl)benzonitrile is crystalline.

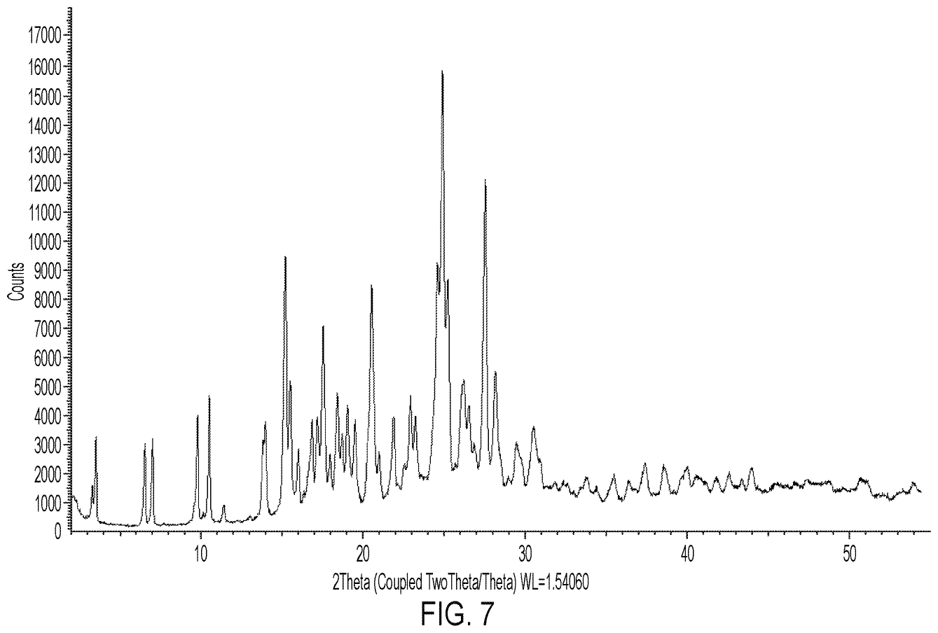

Embodiment D8A1 is a further embodiment of Embodiment D8A, wherein the crystalline hemi sulfate salt of 3-fluoro-4-(((6-(piperidin-4-yl) pyridin-2-yl)oxy)methyl)benzonitrile has a PXRD substantially as FIG. 7.

Embodiment D9 is a further embodiment of Embodiment D1, wherein the present invention provides a crystalline methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate.

Embodiment D9A is a further embodiment of Embodiment D9, wherein the crystalline methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate is Form X and wherein Form X has a powder X-ray diffraction pattern (PXRD) comprising at least two peaks, in terms of 2θ, selected from those at 8.4.0±0.2°, 13.7±0.2°, and 15.0±0.2°.

Embodiment D9A1 is a further embodiment of Embodiment D9A, wherein Form X has a PXRD comprising at least three peaks, in terms of 2θ, at 8.4.0±0.2°, 13.7±0.2°, and 15.0±0.2°.

Embodiment D9A2 is a further embodiment of Embodiment D9A, wherein Form X has a PXRD comprising at least two peaks, in terms of 2θ, selected from those at 8.4.0±0.2°, 11.9±0.2°, 13.7±0.2°, and 15.0±0.2°, and 19.2±0.2°.

Embodiment D9A3 is a further embodiment of Embodiment D9A, wherein Form X has a PXRD comprising at least three peaks, in terms of 2θ, selected from those at. 4.0±0.2°, 11.9±0.2°, 13.7±0.2°, and 15.0±0.2°, and 19.2±0.2°.

Embodiment D9A4 is a further embodiment of Embodiment D9A, wherein Form X has a PXRD comprising peaks, in terms of 2θ, at. 4.0±0.2°, 11.9±0.2°, 13.7±0.2°, and 15.0±0.2°, and 19.2±0.2°.

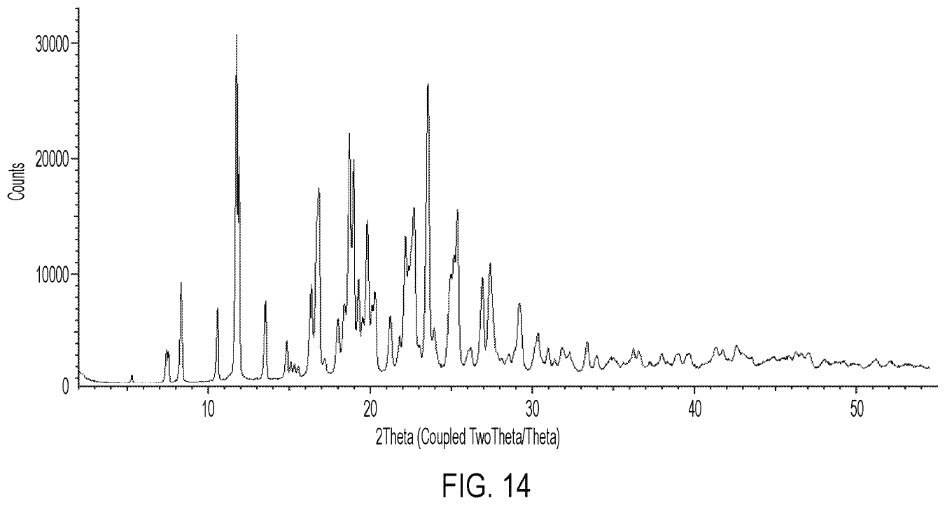

Embodiment D9A5 is a further embodiment of Embodiment D9A, wherein Form X has a PXRD substantially as FIG. 14.

Embodiment D10 is a further embodiment of Embodiment D1, wherein the present invention provides hemi-barium salt of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid, the structure of which is shown below:

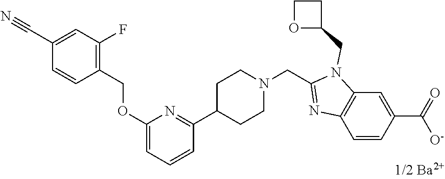 [SMILES: N#Cc1ccc(COc2cccc(C3CCN(Cc4nc5ccc(C(=O)[O-])cc5n4C[C@@H]4CCO4)CC3)n2)c(F)c1]

Embodiment D10A1 is a further embodiment of Embodiment D10, wherein the hemi-barium salt of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid is crystalline.

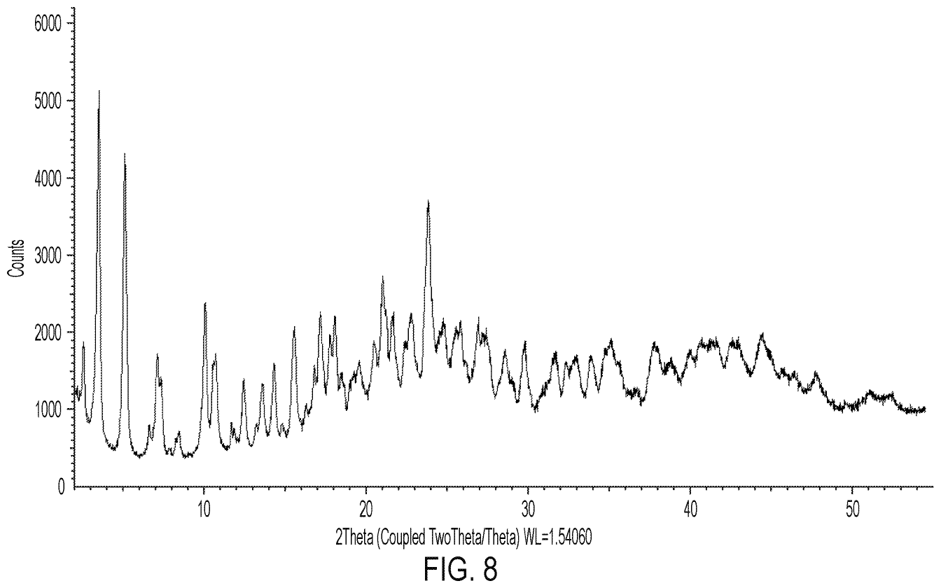

Embodiment D10A2 is a further embodiment of Embodiment D10A1, wherein the crystalline hemi-barium salt of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid has a powder X-ray diffraction pattern (PXRD) substantially as FIG. 8.

In one embodiment (Embodiment E1), the present invention provides a process for preparing methyl(S)-2-(chloromethyl)-1-(oxetan-2-ylmethyl)-1H-benzo[d]imidazole-6-carboxylate:

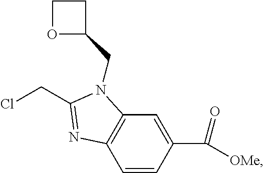 [SMILES: COC(=O)c1ccc2nc(CCl)n(C[C@@H]3CCO3)c2c1]

which process comprising:
(a1) providing methyl(S)-4-amino-3-(oxetane-2-carboxamido)benzoate;
(a2) reducing the methyl(S)-4-amino-3-(oxetane-2-carboxamido)benzoate in the presence of a reducing reagent to form methyl(S)-4-amino-3-((oxetan-2-ylmethyl)amino)benzoate; and
(a3) reacting the methyl(S)-4-amino-3-((oxetan-2-ylmethyl)amino)benzoate with 2-chloro-1,1,1-trimethoxyethane in the presence of an acid (e.g. an organic acid such as citric acid or p-toluenesulfonic acid) to form methyl(S)-2-(chloromethyl)-1-(oxetan-2-ylmethyl)-1H-benzo[d]imidazole-6-carboxylate.

The progress of the reaction in each of Steps (a2) and (a3) can be monitored by a variety of techniques, for example by chromatographic techniques (e.g., LC, TLC, or reverse phase HPLC). The reaction in each of Steps (a2) and (a3) is carried out for a time and under conditions sufficient to form the intended product (and to allow the reaction to go completion).

In some further embodiments of Embodiment E1, methyl(S)-4-amino-3-(oxetane-2-carboxamido)benzoate can be prepared by a method similar to that described in Example 5 hereinafter. Ethyl(S)-oxetane-2-carboxylate is hydrolyzed in the presence of a base (for example, an inorganic strong base such as alkali metal hydroxide, e.g. KOH or NaOH) to form to a salt such as potassium salt of(S)-oxetane-2-carboxylic acid. Triethylamine hydrochloride can be used to treat the potassium salt of(S)-oxetane-2-carboxylic acid to form triethylamine salt of(S)-oxetane-2-carboxylic acid. Then triethylamine salt of(S)-oxetane-2-carboxylic acid can be reacted with methyl 3,4-diaminobenzoate in the presence of a base (e.g. diisopropylethylamine) to form methyl(S)-4-amino-3-(oxetane-2-carboxamido)benzoate.

Embodiment E2 is a further embodiment of Embodiment E1, wherein the reducing reagent in Step (a2) comprises a metal borohydride such as lithium borohydride or sodium borohydride. In some further embodiments, the reducing reagent comprises lithium borohydride. In some yet further embodiments, the amount of the metal borohydride is about 1.0 to about 1.5 (e.g. about 1.25) molar equivalents to the amount of the methyl(S)-4-amino-3-(oxetane-2-carboxamido)benzoate in Step (a2).

Embodiment E3 is a further embodiment of Embodiment E1 or E2, wherein the reducing reagent in Step (a2) comprises a metal borohydride such as lithium borohydride or sodium borohydride; and wherein the reduction reaction is carried out in the presence of a borate ester compound (for example, a trialkyl borate such as triethyl borate). In some embodiments, the amount of the borate ester (e.g. triethyl borate) is about 2.5 to about 3.5 (e.g. 3.0) molar equivalents to the amount of the methyl(S)-4-amino-3-(oxetane-2-carboxamido)benzoate in Step (a2).

Embodiment E4 is a further embodiment of any one Embodiments E1 to E3, wherein the reducing reagent in Step (a2) comprises a metal borohydride such as lithium borohydride or sodium borohydride; and wherein the reduction reaction is carried out in the presence of a borate ester compound (for example, a trialkyl borate such as triethyl borate). In some embodiments, the amount of the borate ester (e.g. triethyl borate) is about 2.5 to about 3.5 (e.g. 3.0) molar equivalents to the amount of the methyl(S)-4-amino-3-(oxetane-2-carboxamido)benzoate in Step (a2).

The progress of the reaction in Step (a2) can be monitored by a variety of techniques, for example by chromatographic techniques (e.g., LC, TLC, or reverse phase HPLC). Suitable organic solvent such as dichloromethane can be used to carried out the reaction. The reaction is carried out at a suitable temperature, for example, at about 10° C. to about 35° C. (e.g. 20° C. or room/ambient temperature). Work-up can be carried out by quenching the reaction mixture (after reaction completion) with water and separating the organic phase from the aqueous phase. The organic phase can be washer with acidic aqueous solution [e.g. phosphoric acid solution (0.5M) and/or aqueous citric acid solution (0.5M)].

The organic phase containing the methyl(S)-4-amino-3-((oxetan-2-ylmethyl)amino)benzoate is concentrated by evaporating the solvents, for example, under vacuum (to remove most or all of the solvents). The residue can be used in Step (a3).

Embodiment E5 is a further embodiment of any one Embodiments E1 to E4, wherein the amount of 2-chloro-1,1,1-trimethoxyethane in Step (a3) is about 1.00 to 1.1 (e.g. 1.02 to about 1.07 such as 1.05) molar equivalents to the amount of to the amount of the methyl(S)-4-amino-3-(oxetane-2-carboxamido)benzoate in Step (a2).

Embodiment E6 is a further embodiment of any one Embodiments E1 to E5, wherein the reaction in Step (a3) is carried out in a solvent system that comprises isopropanol.

Embodiment E7 is a further embodiment of any one Embodiments E1 to E6, wherein the amount of the acid is Step (a3) is about less about 1.5 molar % equivalent (or less than 1.2 molar %, or about 1.0 molar %) of the amount of the methyl(S)-4-amino-3-(oxetane-2-carboxamido)benzoate in Step (a2). In some further embodiments, the acid is citric acid.

Embodiment E8 is a further embodiment of any one Embodiments E1 to E7, wherein the reaction in Step (a3) is carried out at about 40° C. to about 60° C. (e.g. 50° C.).

Embodiment E9 is a further embodiment of any one Embodiments E1 to E8, further comprising isolating the methyl(S)-2-(chloromethyl)-1-(oxetan-2-ylmethyl)-1H-benzo[d]imidazole-6-carboxylate formed in Step (a3).

Reaction in Step (a3) can be worked out as follows [when the solvent comprises or is isopropanol (IPA)]. After reaction completion, the reaction mixture is cooled to about 30° C. or below. Then water is added to form a suspension. The suspension is then cooled to about 5° C., followed by filtration and washing with IPA/Water (e.g. 1:9 vol ratio). The solid collected by filtration is dried, for example, under vacuum and at a temperature of about 30° C. to 50° C. (e.g. 40° C.).

In one embodiment (Embodiment F1), the present invention provides a process for preparing methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate,

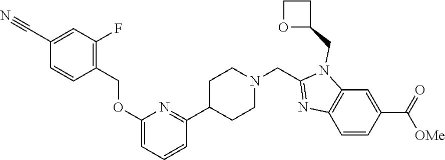 [SMILES: COC(=O)c1ccc2nc(CN3CCC(c4cccc(OCc5ccc(C#N)cc5F)n4)CC3)n(C[C@@H]3CCO3)c2c1]

which process comprising:
(a1) reacting methyl(S)-2-(chloromethyl)-1-(oxetan-2-ylmethyl)-1H-benzo[d]imidazole-6-carboxylate with bis(4-methylbenzenesulfonate) salt of 3-fluoro-4-(((6-(piperidin-4-yl) pyridin-2-yl)oxy)methyl)benzonitrile in the presence diisopropylethylamine in a solvent system comprising acetonitrile, to form the methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate, wherein the amount of the bis(4-methylbenzenesulfonate) salt of 3-fluoro-4-(((6-(piperidin-4-yl) pyridin-2-yl)oxy)methyl)benzonitrile is about 1.1 to about 1.5 molar equivalents to the methyl(S)-2-(chloromethyl)-1-(oxetan-2-ylmethyl)-1H-benzo[d]imidazole-6-carboxylate, and wherein the amount of the diisopropylethylamine is about 4.0 to about 6.0 (e.g. 5.0) molar equivalents to the methyl(S)-2-(chloromethyl)-1-(oxetan-2-ylmethyl)-1H-benzo[d]imidazole-6-carboxylate.

The progress of the reaction in Step (a1) can be monitored by a variety of techniques, for example by chromatographic techniques (e.g., LC, TLC, or reverse phase HPLC). The reaction in Step (a1) is carried out for a time and under conditions sufficient to form the intended product (and to allow the reaction to go completion).

Embodiment F2 is a further embodiment of Embodiment F1, wherein the amount of the solvent system is about 7 ml/g to about 9 ml/g (e.g. 8 ml/g) based on the amount of the methyl(S)-2-(chloromethyl)-1-(oxetan-2-ylmethyl)-1H-benzo[d]imidazole-6-carboxylate.

Embodiment F3 is a further embodiment of Embodiment F1 or F2, wherein the process comprises:
(b1) adding the total amount of the solvent system to a reactor, wherein the solvent system is held at a temperature of about 20° C. to about 30° C. (designated as “the holding temperature”, e.g. 25° C.), and wherein the amount of the solvent system is about 8 ml/g based on the amount of the methyl(S)-2-(chloromethyl)-1-(oxetan-2-ylmethyl)-1H-benzo[d]imidazole-6-carboxylate;
(b2) adding a portion (e.g. ½) of the total amount of the bis(4-methylbenzenesulfonate) salt of 3-fluoro-4-(((6-(piperidin-4-yl) pyridin-2-yl)oxy)methyl)benzonitrile to the reactor, while maintaining the temperature within 5° C. from the holding temperature in Step (b1);
(b3) adding a portion of the total amount of diisopropylethylamine to the reactor over a period of time, while maintaining the temperature within 5° C. from the holding temperature in Step (b1), wherein the portion of the total amount of diisopropylethylamine is the same as the portion of the total amount of the bis(4-methylbenzenesulfonate) salt of 3-fluoro-4-(((6-(piperidin-4-yl) pyridin-2-yl)oxy)methyl)benzonitrile in step (b2);
(b4) repeat Steps (b2) and (b3) until the total amount of the bis(4-methylbenzenesulfonate) salt of 3-fluoro-4-(((6-(piperidin-4-yl) pyridin-2-yl)oxy)methyl)benzonitrile and the diisopropylethylamine is added to the reactor;
(b5) adding the total amount of the methyl(S)-2-(chloromethyl)-1-(oxetan-2-ylmethyl)-1H-benzo[d]imidazole-6-carboxylate to the reactor;
(b6) heating the reaction mixture to a temperature of about 45° C. to about 60° C. (e.g. 50° C.±5.0° C.), and maintaining the reaction mixture at that temperature for a time sufficient to form the methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate;
(b7) seeding the reaction mixture with a seed material of methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate, wherein the amount of the seed material is about 1 weight % of the methyl(S)-2-(chloromethyl)-1-(oxetan-2-ylmethyl)-1H-benzo[d]imidazole-6-carboxylate, wherein the reaction mixture, after seeding, is maintained at a temperature of about 45° C. to about 60° C. for a period of time of greater than about 1 minute;
(b8) adding water [wherein the amount of the water is about 11 ml/g to about 13 ml/g (e.g. 12 ml/g) based on the amount of the methyl(S)-2-(chloromethyl)-1-(oxetan-2-ylmethyl)-1H-benzo[d]imidazole-6-carboxylate] to the reaction mixture over a period of time while maintaining the temperature at about 40° C. to about 55° C.; (b9) cooling the reaction mixture to 15° C.±5.0° C. over about 3 hours (rate of about 0.2° C./min);
(b10) stirring the slurry resulting from Step (b9) at 15° C.±5.0° C. for at least 5 hours (e.g. at least 8 hours);
(b11) filtering the slurry from Step (b10);
(b12) washing the reaction vessel with water and acetonitrile, wherein the wash is cooled to 15° C.±5.0° C.;
(b13) transferring the wash from Step (b12) onto the filter; (b14) collecting the solid from the filtration [Step (b13)]; and (b15) drying the solid collected, optionally under vacuum at about 45° C. to about 55° C. (e.g. 50° C.).

In one embodiment (Embodiment G1), the present invention provides a process for preparing methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate,

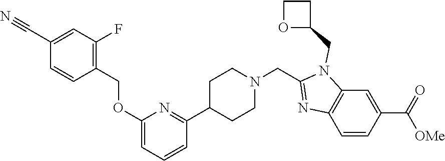 [SMILES: COC(=O)c1ccc2nc(CN3CCC(c4cccc(OCc5ccc(C#N)cc5F)n4)CC3)n(C[C@@H]3CCO3)c2c1]

which process comprising:
(a1) reacting methyl(S)-2-(chloromethyl)-1-(oxetan-2-ylmethyl)-1H-benzo[d]imidazole-6-carboxylate with bis(4-methylbenzenesulfonate) salt of 3-fluoro-4-(((6-(piperidin-4-yl) pyridin-2-yl)oxy)methyl)benzonitrile in the presence diisopropylethylamine in a solvent system comprising methanol, to form the methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate, wherein the amount of the bis(4-methylbenzenesulfonate) salt of 3-fluoro-4-(((6-(piperidin-4-yl) pyridin-2-yl)oxy)methyl)benzonitrile is about 1.0 to about 1.2 (e.g. 1.1) molar equivalents to the methyl(S)-2-(chloromethyl)-1-(oxetan-2-ylmethyl)-1H-benzo[d]imidazole-6-carboxylate, and wherein the amount of the diisopropylethylamine is about 4.0 to about 6.0 (e.g. 5.0) molar equivalents to the methyl(S)-2-(chloromethyl)-1-(oxetan-2-ylmethyl)-1H-benzo[d]imidazole-6-carboxylate.

The progress of the reaction in Step (a1) can be monitored by a variety of techniques, for example by chromatographic techniques (e.g., LC, TLC, or reverse phase HPLC). The reaction in Step (a1) is carried out for a time and under conditions sufficient to form the intended product (and to allow the reaction to go completion).

Embodiment G2 is a further embodiment of Embodiment G1, wherein the amount of the solvent system is about 11 ml/g to about 13 ml/g (e.g. 12 ml/g) based on the amount of the methyl(S)-2-(chloromethyl)-1-(oxetan-2-ylmethyl)-1H-benzo[d]imidazole-6-carboxylate.

Embodiment G3 is a further embodiment of Embodiment G1 or G2, wherein the process comprises:
(b1) adding the total amount of the solvent system to a reactor, wherein the solvent system is held at a temperature of about 20° C. to about 30° C. (designated as “the holding temperature”, e.g. 25° C.), and wherein the amount of the solvent system is about 11 ml/g to about 13 ml/g (e.g. 12 ml/g) based on the amount of the methyl(S)-2-(chloromethyl)-1-(oxetan-2-ylmethyl)-1H-benzo[d]imidazole-6-carboxylate;
(b2) adding the total amount of the bis(4-methylbenzenesulfonate) salt of 3-fluoro-4-(((6-(piperidin-4-yl) pyridin-2-yl)oxy)methyl)benzonitrile to the reactor, while maintaining the temperature within 5° C. from the holding temperature in Step (b1);
(b3) adding the total amount of diisopropylethylamine to the reactor over a period of time, while maintaining the temperature within 5° C. from the holding temperature in Step (b1), (b4) adding the total amount of the methyl(S)-2-(chloromethyl)-1-(oxetan-2-ylmethyl)-1H-benzo[d]imidazole-6-carboxylate to the reactor;
(b5) heating the reaction mixture to a temperature of about 45° C. to about 60° C. (e.g. 50° C.±5.0° C.), and maintaining the reaction mixture at that temperature for a time sufficient to form the methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate;
(b6) seeding the reaction mixture with a seed material of methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate, wherein the amount of the seed material is about 1 weight % of the methyl(S)-2-(chloromethyl)-1-(oxetan-2-ylmethyl)-1H-benzo[d]imidazole-6-carboxylate, wherein the reaction mixture, after seeding, is maintained at a temperature of about 45° C. to about 60° C. for a period of time of greater than about 1 minute;
(b7) adding water [wherein the amount of the water is about 3 ml/g to about 5 ml/g (e.g. 4 ml/g) based on the amount of the methyl(S)-2-(chloromethyl)-1-(oxetan-2-ylmethyl)-1H-benzo[d]imidazole-6-carboxylate] to the reaction mixture over a period of time while maintaining the temperature at about 40° C. to about 55° C.;
(b8) cooling the reaction mixture to 20° C.±5.0° C. over at least two hours (rate of about 0.2° C./min);
(b9) stirring the slurry resulting from Step (b8) at 15° C.±5.0° C. for at least 2 hours (e.g. at least 3 hours);
(b10) filtering the slurry from Step (b9);
(b11) washing the reaction vessel with water and methanol, wherein the wash is cooled to 20° C.±5.0° C.;
(b12) transferring the wash from Step (b11) onto the filter;
(b13) collecting the solid from the filtration [Step (b12)]; and
(b14) drying the solid collected, optionally under vacuum at about 45° C. to about 55° C. (e.g. 50° C.).

In one embodiment (Embodiment H1), the present invention provides a process for preparing hemi-barium salt of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid

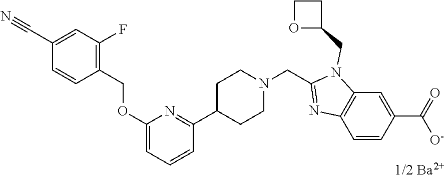 [SMILES: N#Cc1ccc(COc2cccc(C3CCN(Cc4nc5ccc(C(=O)[O-])cc5n4C[C@@H]4CCO4)CC3)n2)c(F)c1]

which process comprising:
(a1) reacting methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate with barium hydroxide [Ba(OH)₂] in a solvent system comprising an organic solvent and water, to form hemi-barium salt of (S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid, wherein the amount of barium hydroxide [Ba(OH)₂] is about 0.5 to about 0.6 (e.g. about 0.5) molar equivalent to the methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate; and
(a2) optionally isolating the hemi-barium salt of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid from Step (a1).

The progress of the reaction in Step (a1) can be monitored by a variety of techniques, for example by chromatographic techniques (e.g., LC, TLC, or reverse phase HPLC). The reaction in Step (a1) is carried out for a time and under conditions sufficient to form the intended product (and to allow the reaction to go completion).

Embodiment H2 is a further embodiment of Embodiment H1, wherein the organic solvent in the solvent system in Step (a1) comprises an aprotic organic solvent, wherein the aprotic organic solvent is miscible with water. In some further embodiments, the organic solvent in the solvent system is acetone or acetonitrile. In some yet further embodiments, the volume ratio of the organic solvent and water in the solvent system is from about 4.5:1 to about 5.5:1, or from about 4.5:1 to about 5.0:1, such as about 4.7:1, about 4.8:1, or about 5:1.

Embodiment H3 is a further embodiment of Embodiment H1 or H2, wherein the organic solvent in the solvent system in Step (a1) is acetonitrile, and wherein the volume ratio of the organic solvent and water in the solvent system is from about 4.5:1 to about 5.0:1.

Embodiment H4 is a further embodiment of any one of Embodiments H1 to H3, wherein the organic solvent in the solvent system in Step (a1) is acetonitrile; wherein the volume ratio of the organic solvent and water in the solvent system is from about 4.5:1 to about 5.0:1; and wherein the volume amount of the acetonitrile is from about 5 ml/g to about 15 ml/g based on the amount of the methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate.

Embodiment H5 is a further embodiment of any one of Embodiments H1 to H4, wherein the reaction in Step (a1) is carried out at an elevated temperature, for example, at a temperature of about 40° C. to about 65° C. (e.g. 50° C. or 60° C.).

Embodiment H6 is a further embodiment of Embodiment H1 or H2, wherein the organic solvent in the solvent system is acetone, and wherein the volume ratio of the organic solvent and water in the solvent system is from about 4.5:1 to about 5.5:1 (e.g. 5.0:1).

Embodiment H7 is a further embodiment of Embodiment H6, wherein the volume amount of the acetone is about 5 ml/g to about 15 ml/g (e.g. 6 ml/g) based on the amount of the methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate.

Embodiment H8 is a further embodiment of Embodiment H6 or H7, wherein the reaction in Step (a1) is carried out at an elevated temperature, for example, at a temperature of about 45° C. to about 55° C. (e.g. 50° C.).

The progress of the reaction in Step (a1) can be monitored by a variety of techniques, for example by chromatographic techniques (e.g., LC, TLC, or reverse phase HPLC). Upon reaction completion, the reaction mixture can be cooled from the elevated temperature to room/ambient temperature. The hemi-barium salt of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid formed precipitates from reaction solvent system.

Embodiment H9 is a further embodiment of any one of Embodiments H1 to H8, wherein isolating the hemi-barium salt of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid in Step (a2) comprises filtering the hemi-barium salt of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid formed.

Embodiment H10 is a further embodiment of any one of Embodiments H1 to H8, wherein the isolating the hemi-barium salt of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid in Step (a2) comprises filtering the hemi-barium salt of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid formed; and washing the hemi-barium salt of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid obtained by filtration with the same organic solvent as that in the organic solvent system in Step (a1).

Embodiment H11 is a further embodiment of any one of Embodiments H1 to H10, wherein the isolated hemi-barium salt of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid in Step (a2) is further dried, optionally under vacuum and at an elevated temperature (e.g. 45° C. to about 55° C.).

In one embodiment (Embodiment J1), the present invention provides a process for preparing tris salt of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid,

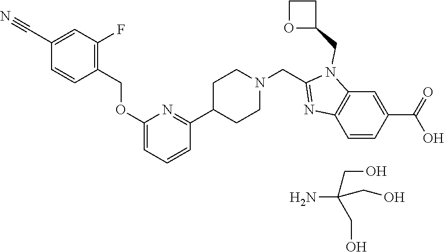

which process comprising:
(a1) reacting methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate with barium hydroxide [Ba(OH)₂] in a solvent system comprising acetonitrile and water, to form hemi-barium salt of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid, wherein the amount of barium hydroxide [Ba(OH)₂] is about 0.5 to about 0.6 (e.g. about 0.5) molar equivalent to the methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate;
(a2) adding water, a water-immiscible organic solvent (e.g. toluene, TBME, or Ethyl acetate), and an organic acid (e.g. acetic acid) to the reaction mixture in Step (a1), and mixing the resultant mixture to form(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid;
(a3) separating the organic phase/solution from the aqueous phase/solution from the mixture from Step (a2);
(a4) adding 2-amino-2-(hydroxymethyl)-1,3-propanediol to the separated organic phase from Step (a3) whereby reacting 2-amino-2-(hydroxymethyl)-1,3-propanediol and the(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid, to form tris salt of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid, wherein the reaction mixture is held at a holding temperature of about 35° C. to about 55° C. (e.g. about 40° C. to about 50° C., or about 45° C.);
(a5) adding a seed crystalline material of tris salt of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid to the reaction mixture from Step (a4) to form a slurry, wherein the slurry is held at the holding temperature for a period of time of greater than about 1 minute;
(a6) cooling the slurry from Step (a5) to a temperature of about 20° C. to about 30° C. (e.g. about 25° C.), and holding the slurry at that temperature for a period of time of greater than about 1 minute;
(a7) optionally heating the slurry from Step (a6) to the holding temperature [about 40° C. to about 50° C., e.g. about 45° C.] and holding the slurry at the holding temperature for a period of time of greater than about 1 minute;
(a8) optionally cooling the slurry from Step (a7) to a temperature of about 15° C. to about 25° C. (e.g. 20° C.), and holding the slurry at that temperature for a period of time of greater than about 1 minute; and
(a9) isolating the tris salt of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid in the slurry from Step (a6) or (a8).

The progress of the reaction in each of Steps (a1), (a2), and (a4) can be monitored by a variety of techniques, for example by chromatographic techniques (e.g., LC, TLC, or reverse phase HPLC). The reaction in each of Steps (a1) and (a4) is carried out for a time and under conditions sufficient to form the intended product (and to allow the reaction to go completion).

Embodiment J2 is a further embodiment of Embodiment J1, wherein the volume ratio of the acetonitrile and water in the solvent system in Step (a1) is from about 4.5:1 to about 5.5:1, or from about 4.5:1 to about 5.0:1, such as about 4.7:1, about 4.8:1, or about 5:1.

Embodiment J3 is a further embodiment of Embodiment J1 or J2, wherein the volume amount of the acetonitrile in Step (a1) is from about 5 ml/g to about 10 ml/g (e.g. about 7 ml/g) based on the amount of the methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate. In some further embodiments, the volume amount of the acetonitrile in Step (a1) is from about 6 ml/g to about 8 ml/g (e.g. about 7 ml/g) based on the amount of the methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate.

Embodiment J4 is a further embodiment of any one of Embodiments J1 to J3, wherein the reaction in Step (a1) is carried out at an elevated temperature, for example, at a temperature of about 40° C. to about 65° C. (e.g. about 50° C. or about 60° C.).

The progress of the reaction in Step (a1) can be monitored by a variety of techniques, for example by chromatographic techniques (e.g., LC, TLC, or reverse phase HPLC). Upon reaction completion, the reaction mixture can be cooled from the elevated temperature to a lower temperature such as room/ambient temperature, or a temperature from about 20° C. to about 25° C. The hemi-barium salt of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid formed precipitates from reaction solvent system, and the reaction mixture is in the form of a slurry. This slurry is carried over directly to Step (a2).

Embodiment J5 is a further embodiment of any one of Embodiments J1 to J4, wherein the volume amount of the water added in Step (a2) is from about 3 ml/g to about 4 ml/g (e.g. about 3.5 ml/g) based on the amount of the methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate used in Step (a1).

Embodiment J6 is a further embodiment of any one of Embodiments J1 to J5, wherein the volume amount of the water-immiscible organic solvent (e.g. toluene) added in Step (a2) is about 0.8 ml/g or greater, such as from 0.8 ml/g to about 1.2 ml/g (e.g. about 1.0 ml/g) based on the amount of the methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate used in Step (a1).

Embodiment J7 is a further embodiment of any one of Embodiments J1 to J6, wherein the amount of the organic acid (e.g. acetic acid) added in Step (a2) is from about 1.0 to about 2.0 (e.g. about 1.5) molar equivalents of the methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate used in Step (a1). In some further embodiments, the organic acid added in Step (a2) is acetic acid.

In Step (a2), water and toluene are added to the reaction mixture from Step (a1). An organic acid (e.g. acetic acid) is added to the mixture to covert the hemi-barium salt of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid to the free acid of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid. The free acid is extracted into the organic phase that includes the water-immiscible organic solvent (e.g. toluene).

In Step (3), the organic phase that includes the water-immiscible organic solvent (e.g. toluene) from Step (a2) is separated from the aqueous phase. The Organic phase is carried over to Step (a4) directly.

Embodiment J8 is a further embodiment of any one of Embodiments J1 to J7, wherein the amount of the 2-amino-2-(Hydroxymethyl)-1,3-propanedio added in Step (a4) is from about 1.0 to about 1.5 (e.g. from about 1.1 to about 1.3, or about 1.2) molar equivalents of the methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate used in Step (a1).

Embodiment J9 is a further embodiment of any one of Embodiments J1 to J8, wherein the 2-amino-2-(Hydroxymethyl)-1,3-propanedio added in Step (a4) is in the form of an aqueous solution.

Embodiment J10 is a further embodiment of any one of Embodiments J1 to J9, wherein the reaction in Step (a4) is carried out at an elevated temperature of about 45° C.

Embodiment J11 is a further embodiment of any one of Embodiments J1 to J10, wherein the amount of the seed is about 0.20 to about 0.50 molar % (e.g., about 0.25 to about 0.35 molar %, or about 0.3 molar %) of the amount of the methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate used in Step (a1) [e.g. about 0.004 g/g based on the amount of the methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate used in Step (a1).

Embodiment J12 is a further embodiment of any one of Embodiments J1 to J11, wherein Steps (a7) and (a8) are carried out. Steps (a7) and (a8) reduce some impurities, for example, methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate.

Embodiment J13 is a further embodiment of any one of Embodiments J1 to J12, wherein isolating the tris salt of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid in the slurry from Step (a6) or (a8) comprises filtration, for example, filtration under vacuum.

Embodiment J14 is a further embodiment of Embodiment J13, wherein the filtration or the filtration under vacuum further comprises washing with the solid with methyl ethyl ketone (MEK). In some further embodiments, washing with MEK comprises 1, 2, or 3 washings. In some further embodiments, the amount of the MEK used for each washing is about 2 mL/g to about 4 mL/g (e.g. about 3 mL/g) based on the amount of the methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate used in Step (a1).

Embodiment J15 is a further embodiment of any one of Embodiments J1 to J14, wherein the isolated tris salt of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid from Step (a9) is further dried, for example, dried under vacuum at a temperature of no more than about 40° C., no more than about 50° C., no more than about 60° C., or from about 45° C. to about 55° C. (e.g. 50° C.).

Embodiment J16 is a further embodiment of any one of Embodiments J1 to J15, wherein the slurry in Step (a5) is held at the holding temperature for a period of time greater than about 1 minute, greater than about 5 minutes, greater than about 10 minutes, greater than about 15 minutes, greater than about 20 minutes, greater than about 30 minutes, greater than about 45 minutes, or greater than about 60 minutes.

Embodiment J17 is a further embodiment of any one of Embodiments J1 to J16, wherein the slurry in Step (a6) is held at the temperature of about 20° C. to about 30° C. for a period of time greater than about 1 minute, greater than about 5 minutes, greater than about 10 minutes, greater than about 15 minutes, greater than about 20 minutes, greater than about 30 minutes, greater than about 45 minutes, or greater than about 60 minutes.

Embodiment J18 is a further embodiment of any one of Embodiments J1 to J17, wherein the slurry in Step (a7) is held at the holding temperature for a period of time greater than about 1 minute, greater than about 5 minutes, greater than about 10 minutes, greater than about 15 minutes, greater than about 20 minutes, greater than about 30 minutes, greater than about 45 minutes, or greater than about 60 minutes.

Embodiment J19 is a further embodiment of any one of Embodiments J1 to J18, wherein the slurry in Step (a8) is held at the temperature of about 15° C. to about 25° C. for a period of time greater than about 1 minute, greater than about 5 minutes, greater than about 10 minutes, greater than about 15 minutes, greater than about 20 minutes, greater than about 30 minutes, greater than about 45 minutes, greater than about 60 minutes, greater than about 1 hour, greater than about 2 hours, greater than about 3 hours, greater than about 4 hours, greater than about 5 hours, greater than about 6 hours, greater than about 7 hours, greater than about 8 hours, or greater than about 9 hours. In some further embodiments, the holding time in Step (a8) is greater than about 4 hours, greater than about 5 hours, greater than about 6 hours, greater than about 7 hours, or greater than about 8 hours.

In one embodiment (Embodiment K1), the present invention provides a process for preparing tris salt of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid

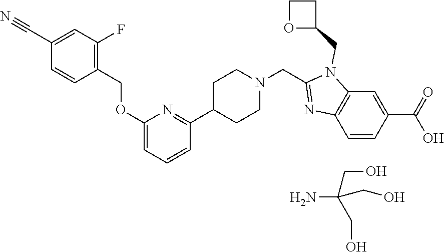

which process comprising:
(a1) reacting methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate with barium hydroxide [Ba(OH)₂] in a solvent system comprising acetone and water, to form hemi-barium salt of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid, wherein the amount of barium hydroxide [Ba(OH)₂] is about 0.5 to about 0.6 (e.g. about 0.5) molar equivalent to the methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate;
(a2) adding water, a water-immiscible organic solvent (e.g. toluene, TBME, or Ethyl acetate), and an organic acid (e.g. acetic acid) to the reaction mixture from Step (a1), and mixing the resultant mixture to form(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylic acid;
(a3) separating the organic phase from the aqueous phase from the mixture from Step (a2) and optionally adding methanol to the separated organic phase;
(a4) adding 2-amino-2-(hydroxymethyl)-1,3-propanediol to the separated organic phase from Step (a3) whereby reacting 2-amino-2-(hydroxymethyl)-1,3-propanediol and the(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid, to form tris salt of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid, wherein the reaction mixture is held at a holding temperature of about 35° C. to about 55° C. (e.g. about 40° C. to about 50° C., or about 45° C.);
(a5) adding a seed crystalline material of tris salt of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid to the reaction mixture from Step (a4) to form a slurry, wherein the slurry is held at the holding temperature for a period of time of greater than about 1 minute;
(a6) cooling the slurry from Step (a5) to a temperature of about 15° C. to about 20° C. (e.g. 20° C.), and holding the slurry at that temperature for a period of time of greater than about 1 minute; and
(a7) isolating the tris salt of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid from the slurry from Step (a6).

The progress of the reaction in each of Steps (a1), (a2), and (a4) can be monitored by a variety of techniques, for example by chromatographic techniques (e.g., LC, TLC, or reverse phase HPLC). The reaction in each of Steps (a1) and (a4) is carried out for a time and under conditions sufficient to form the intended product (and to allow the reaction to go completion).

Embodiment K2 is a further embodiment of Embodiment K1, wherein the volume ratio of the acetone and water in the solvent system in Step (a1) is from about 4.5:1 to about 5.5:1, such as about 5:1.

Embodiment K3 is a further embodiment of Embodiment K1 or K2, wherein the volume amount of the acetone in Step (a1) is from about 5 ml/g to about 8 ml/g (e.g. about 6 ml/g) based on the amount of the methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate. In some further embodiments, the volume amount of the acetonitrile in Step (a1) is from about 5 ml/g to about 7 ml/g (e.g. about 6 ml/g) based on the amount of the methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate.

Embodiment K4 is a further embodiment of any one of Embodiments K1 to K3, wherein the reaction in Step (a1) is carried out at an elevated temperature, for example, at a temperature of from about 40° C. to about 60° C. (e.g. from about 45° C. to about 55° C., or about 50° C.).

Embodiment K5 is a further embodiment of any one of Embodiments K1 to K4, wherein the volume amount of the water added in Step (a2) is from about 2.5 ml/g to about 4 ml/g (e.g. about 3.0 ml/g) based on the amount of the methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate used in Step (a1).

Embodiment K6 is a further embodiment of any one of Embodiments K1 to K5, wherein the volume amount of the water-immiscible organic solvent (e.g. toluene, TBME, or ethyl acetate) added in Step (a2) is about 2.0 ml/g or greater, such as from 2.0 ml/g to about 4.0 ml/g, or from 3.0 ml/g to about 4.0 ml/g (e.g. about 3.5 ml/g) based on the amount of the methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate used in Step (a1). In some further embodiments, the water-immiscible organic solvent is toluene.

Embodiment K7 is a further embodiment of any one of Embodiments K1 to K6, wherein the amount of the organic acid (e.g. acetic acid) added in Step (a2) is from about 1.0 to about 2.0 (e.g. about 1.5) molar equivalents of the methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate used in Step (a1). In some further embodiments, the organic acid added in Step (a2) is acetic acid.

The progress of the reaction in Step (a1) can be monitored by a variety of techniques, for example by chromatographic techniques (e.g., LC, TLC, or reverse phase HPLC). Upon reaction completion, the reaction mixture can be cooled from the elevated temperature to a lower temperature such as room/ambient temperature, or a temperature from about 20° C. to about 30° C. (e.g. from about 22° C. to about 28° C., or from about 23° C. to about 27° C., or about 25° C.). The hemi-barium salt of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid formed precipitates from reaction solvent system, and the reaction mixture is in the form of a slurry. This slurry is carried over directly to Step (a2).

In Step (a2) of some embodiments, water and toluene are added to the reaction mixture from Step (a1). An organic acid (e.g. acetic acid) is added to the mixture to covert the hemi-barium salt of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid to the free acid of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid. After addition of the organic acid, the reaction mixture in Step (a2) is stirred for a time and under conditions sufficient to complete the reaction (e.g. at a temperature of about 20° C. to about 30° C., such as 25° C.). Then the free acid formed in Step (a2) is extracted into the organic phase that includes the water-immiscible organic solvent (e.g. toluene) in Step (a3).

In Step (a3), the organic phase that includes the water-immiscible organic solvent (e.g. toluene) from Step (a2) is separated from the aqueous phase. After the separation, optionally methanol is added to the organic phase and the resultant solution is carried over to Step (a4) directly.

Embodiment K8 is a further embodiment of any one of Embodiments K1 to K7, wherein the amount of methanol used in Step (a3), if present, is from about 0.5 mL/g to 1.0 mL/g (e.g. 0.75 mL/g) based on the amount of the methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate used in Step (a1).

Embodiment K9 is a further embodiment of any one of Embodiments K1 to K8, wherein the amount of the 2-amino-2-(Hydroxymethyl)-1,3-propanedio added in Step (a4) is from about 1.0 to about 1.5 (e.g. from about 1.1 to about 1.3, or about 1.2) molar equivalents of the methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate used in Step (a1).

Embodiment K10 is a further embodiment of any one of Embodiments K1 to K9, wherein the 2-amino-2-(Hydroxymethyl)-1,3-propanedio added in Step (a4) is in the form of an aqueous solution.

Embodiment K11 is a further embodiment of any one of Embodiments K1 to K10, wherein the reaction in Step (a4) is carried out at an elevated temperature of about 45° C.

Embodiment K12 is a further embodiment of any one of Embodiments K1 to K11, wherein the amount of the seed is about 0.20 to about 0.50 molar % (e.g. about 0.25 to about 0.35 molar %, or about 0.3 molar %) of the amount of the methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate used in Step (a1) [e.g. about 0.004 g/g based on the amount of the methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate used in Step (a1). In some further embodiments, after the seeding is carried out, the slurry is stirred for a period of time greater than about 1 hour.

Embodiment K13 is a further embodiment of any one of Embodiments K1 to K12, wherein isolating the tris salt of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid in Step (a7) comprises filtration, for example, filtration under vacuum.

Embodiment K14 is a further embodiment of Embodiment K13, wherein the filtration or the filtration under vacuum further comprises washing with the solid with acetone. In some further embodiments, washing with acetone comprises 1, 2, or 3 washings (e.g. 2 washings). In some further embodiments, the amount of the acetone used for each washing is about 2 mL/g to about 4 mL/g (e.g. about 3 mL/g) based on the amount of the methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate used in Step (a1).

Embodiment K15 is a further embodiment of any one of Embodiments K1 to K14, wherein the isolated tris salt of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid from Step (a7) is further dried, for example, dried under vacuum at a temperature of no more than about 40° C., of no more than about 50° C., of no more than about 60° C., or from about 45° C. to about 55° C. (e.g. 50° C.).

Embodiment K16 is a further embodiment of any one of Embodiments K1 to K15, wherein the slurry in Step (a5) is held at the holding temperature for a period of time greater than about 1 minute, greater than about 5 minutes, greater than about 10 minutes, greater than about 15 minutes, greater than about 20 minutes, greater than about 30 minutes, greater than about 45 minutes, or greater than about 60 minutes.

Embodiment K17 is a further embodiment of any one of Embodiments K1 to K16, wherein the slurry in Step (a6) is held at the temperature of about 15° C. to about 20° C. for a period of time greater than about 1 minute, greater than about 5 minutes, greater than about 10 minutes, greater than about 15 minutes, greater than about 20 minutes, greater than about 30 minutes, greater than about 45 minutes, greater than about 60 minutes, greater than about 1 hour, greater than about 2 hours, greater than about 3 hours, greater than about 4 hours, greater than about 5 hours, greater than about 6 hours, greater than about 7 hours, greater than about 8 hours, or greater than about 9 hours. In some further embodiments, the holding time in Step (a8) is greater than about 4 hours, greater than about 5 hours, greater than about 6 hours, greater than about 7 hours, or greater than about 8 hours.

In one embodiment (Embodiment L1), the present invention provides a method for preparing Form 1 of tris salt of 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid, which method comprises:
(a) suspending tris salt of 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid in a solvent system, wherein the solvent system consists of dimethyl sulfoxide (DMSO) and water, wherein the volume ratio of DMSO:water is from about 10:1 to about 6:1 (e.g., from about 9:1 to about 7:1, or about 8:1), and wherein the ratio of the water (volume) to the tris salt of 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid (weight) is from about 0.8 mL/g to about 1.2 mL/g (e.g. 1.0 mL/g) at a temperature of from about 20° C. to about 35° C. (e.g. from about 20° C. to about 30° C.);
(b) heating the suspension in Step (a) to an elevated temperature from about 60° C. to about 70° C. (e.g. about 65° C.) to form a solution, and then mixing the solution at the elevated temperature for a period of time of greater than about 1 minute;
(c) adding water to the solution from Step (b) slowly while maintaining the reaction mixture as a solution, wherein the amount of water added is about the same as the water used in Step (a), and then holding the resultant solution at the elevated temperature for a period of time of greater than about 1 minute;
(d) seeding the solution with a crystalline Form 1 material of tris salt of 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid while maintaining the temperature at the elevated temperature, wherein the amount of the seed crystalline is about 0.5 weight % or more (e.g. about 0.5 weight % or about 1.0 weight %) of the tris salt of 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid used in Step (a), and then holding the resultant mixture at the elevated temperature for a period of time of greater than about 1 minute;
(e) optionally cooling the temperature of the mixture from Step (d) to about 30° C. slowly (e.g. at a rate of about 0.2° C./min), and then holding the mixture at that temperature for a period of time of greater than about 1 minute;
(f) optionally heating the mixture from Step (e) to about 40° C. to about 50° C. slowly (e.g. at a rate of about 0.5° C./min), and then holding the mixture at that temperature for a period of time of greater than about 1 minute;
(g) cooling the temperature of the mixture from Step (d) or Step (f) [if Steps (e) and (f) are carried out] to about 15° C. slowly (e.g. at a rate of about 0.2° C./min), and then holding the mixture at that temperature for a period of time of greater than about 1 minute; and
(h) isolating the solid from the resultant mixture from Step (g) to afford the Form I of tris salt of 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid.

Embodiment L1 relates to a recrystallization process wherein (1) a solvent system consisting of dimethyl sulfoxide (DMSO) and water is used to dissolve tris salt of 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid at an elevated temperature; (2) adding water (an anti-solvent) to the solution at the elevated temperature while maintain the resultant mixture as a solution; (3) seeding the solution at the elevated temperature with crystalline Form I materials; (4) cooling the resultant mixture to a low temperature (e.g. about 15° C.); (5) optionally heating up and then cooling the mixture; and (6) isolating the crystalline From I from the cooled mixture.

Embodiment L2 is a further embodiment of Embodiment L1, wherein the volume ratio of DMSO:water in Step (a) is from about 9:1 to about 7:1.

Embodiment L3 is a further embodiment of Embodiment L1 or L2, wherein the volume ratio of DMSO:water in Step (a) is about 8:1.

Embodiment L4 is a further embodiment of any one of Embodiments L1 to L3, wherein the ratio of the water (volume) to the tris salt of 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid (weight) in Step (a) is about 1.0 mL/g.

Embodiment L5 is a further embodiment of any one of Embodiments L1 to L4, wherein the suspension is heated up at an elevated temperature (e.g. about 65° C.) to form a solution in Step (b). In some further embodiments, the solution in Step (b) is mixed at the elevated temperature for a period of time greater than about 1 minute, greater than about 5 minutes, greater than about 10 minutes, greater than about 15 minutes, greater than about 20 minutes, or greater than about 30 minutes.

Embodiment L6 is a further embodiment of any one of Embodiments L1 to L5, wherein water is added in Step (c) slowly so that the mixture maintains as a solution and the temperature of the solution is maintained without deviating from the elevated temperature more than 5.0° C. In some further embodiments, the holding time in Step (c) is greater than about 1 minute, greater than about 5 minutes, greater than about 10 minutes, greater than about 15 minutes, greater than about 20 minutes, or greater than about 30 minutes.

Embodiment L7 is a further embodiment of any one of Embodiments L1 to L6, wherein seeding in Step (d) is carried out so that the temperature of the mixture is maintained without deviating from the elevated temperature more than 5.0° C., and so that at least some seeding material remains as solid after seeding is complete and that some solid exists after the holding period in Step (d). In some further embodiments, the holding time in Step (d) is greater than about 1 minute, greater than about 5 minutes, greater than about 10 minutes, greater than about 15 minutes, greater than about 20 minutes, greater than about 30 minutes, greater than about 45 minutes, or greater than about 60 minutes. In some further embodiments, the holding time in Step (e) is greater than about 1 hour.

Embodiment L8 is a further embodiment of any one of Embodiments L1 to L7, wherein Steps (e) and (f) are carried out.

Embodiment L9 is a further embodiment of any one of Embodiments L1 to L8, wherein the cooling in Step (e) is carried out at a rate of less than about 1° C./min, less than about 0.8° C./min, less than about 0.5° C./min, less than about 0.3° C./min (e.g. about 0.2° C./min). In some further embodiments, the cooling in Step (e) is carried out at a rate of about 0.2° C./min.

Embodiment L10 is a further embodiment of any one of Embodiments L1 to L9, wherein the holding time in Step (e) is greater than about 1 minute, greater than about 5 minutes, greater than about 10 minutes, greater than about 15 minutes, greater than about 20 minutes, greater than about 30 minutes, or greater than about 1 hour. In some further embodiments, the holding time in Step (e) is greater than about 1 hour.

Embodiment L11 is a further embodiment of any one of Embodiments L1 to L10, wherein the heating in Step (f) is carried out at a rate of less than about 1° C./min, less than about 0.8° C./min, less than about 0.7° C./min, less than about 0.6° C./min, less than about 0.5° C./min, less than about 0.3° C./min. In some further embodiments, the heating in Step (f) is carried out at a rate of about 0.5° C./min.

Embodiment L12 is a further embodiment of any one of Embodiments L1 to L11, wherein the holding time in Step (f) is greater than about 1 minute, greater than about 5 minutes, greater than about 10 minutes, greater than about 15 minutes, greater than about 20 minutes, or greater than about 30 minutes. In some further embodiments, the holding time in Step (f) is about 30 minutes.

Embodiment L13 is a further embodiment of any one of Embodiments L1 to L12, wherein the cooling in Step (g) is carried out at a rate of less than about 1° C./min, less than about 0.8° C./min, less than about 0.5° C./min, or less than about 0.3° C./min (e.g. about 0.2° C./min). In some further embodiments, the cooling in Step (g) is carried out at a rate of about 0.2° C./min.

Embodiment L14 is a further embodiment of any one of Embodiments L1 to L13, wherein the holding time in Step (g) is greater than about 1 hour, greater than about 2 hours, greater than about 3 hours, greater than about 4 hours, greater than about 5 hours, greater than about 6 hours, greater than about 7 hours, greater than about 8 hours, or greater than about 9 hours. In some further embodiments, the holding time in Step (g) is greater than about 8 hours.

Embodiment L15 is a further embodiment of any one of Embodiments L1 to L14, wherein isolating the solid in Step (f) comprises filtration, for example, filtration under vacuum.

Embodiment L16 is a further embodiment of Embodiment L15, wherein the filtration or the filtration under vacuum further comprises washing with the solid with methyl ethyl ketone (MEK). In some further embodiments, washing with MEK comprises 1, 2, or 3 washings (e.g. 2 washings). In some yet further embodiments, the amount of the MEK used for each washing is about 2 mL/g to about 4 mL/g (e.g. about 3 mL/g) based on the amount of the methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate used in Step (a1). In still further embodiments, the MEK used for each washing is cooled to about 15° C. before washing.

Embodiment L16 is a further embodiment of any one of Embodiments L1 to L15, wherein the isolated solid from Step (h) is further dried, for example, dried under vacuum at a temperature of no more than about 40° C., of no more than about 50° C., of no more than about 60° C., or from about 50° C. to about 60° C. (e.g. 55° C.).

In one embodiment (Embodiment M1), the present invention provides a method for preparing Form 1 of tris salt of 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid, which method comprises:
(a) suspending tris salt of 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid in a solvent system, wherein the solvent system consists of tetrahydrofuran (THF) and water, wherein the volume ratio of THF:water is from about 1:1 to about 4:1, (e.g. from about 2.5:1 to about 3.5:1, from about 2.8:1 to about 3.2:1, from about 2.9:1 to about 3.1:1, or about 3.0:1), and wherein the ratio of the water (volume) to the tris salt of 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid (weight) is from about 1.1 mL/g to about 3.8 mL/g (e.g. from about 1.3 mL/g to about 1.5 mL/g, or about 1.39 mL/g) at a temperature of from about 20° C. to about 35° C. (e.g. from about 20° C. to about 30° C., about 22° C.);
(b) heating the suspension in Step (a) to a high temperature of from about 49° C. to about 59° C. (e.g. about 55° C.) to form a solution, cooling the temperature to a holding temperature of from about 47° C. to about 51° C. (e.g. about 49° C.) while the mixture remains as a solution, and optionally mixing the solution at the holding temperature for a period of time of greater than about 1 minute;
(c) seeding the solution from Step (b) with a crystalline Form 1 material of tris salt of 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid while maintaining the temperature at the holding temperature, wherein the amount of the seed crystalline is about 0.5 weight % or more (e.g. about 0.5 weight % or about 1.0 weight %) of the tris salt of 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid used in Step (a), and then holding the resultant mixture at the holding temperature for a period of time of greater than about 1 minute;
(d) cooling the temperature of the mixture from Step (c) to an intermediate temperature of about 35° C. slowly, and then holding the mixture at the intermediate temperature for a period of time of greater than about 1 minute;
(e) adding a water-miscible organic solvent (e.g. acetonitrile, isopropanol, or acetone) to the mixture from Step (d) slowly while maintaining the temperature of the mixture at the intermediate temperature, and then holding the mixture at intermediate temperature for a period of time of greater than about 1 minute;
(f) cooling the temperature of the mixture from Step (e) to a low temperature of about 10° C. slowly, and then holding the mixture at the low temperature for a period of time of greater than about 1 minute;
(g) taking a sample of the mixture (slurry) to determine the particle size of the solid in the mixture;
(h) performing high shear wet milling until the D90 of the particle size of the solid in the mixture is less than about 150 μM;
(i) isolating the solid from the resultant mixture from Step (f) to afford the Form I of tris salt of 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid.

Embodiment M1 relates to a recrystallization process, wherein (1) a solvent system consisting of THF and water is used to dissolve tris salt of 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid at a high temperature (e.g. 55° C.) and then cooling the solution to a holding temperature (e.g. 49° C.); (2) seeding the solution at the holding temperature with crystalline Form I materials; (3) cooling the resultant mixture to an intermediate temperature (e.g. 35° C.), and adding acetonitrile at intermediate temperature; (4) cooling the resultant mixture to a low temperature (e.g. about 10° C.); and (5) isolating the crystalline From I from the cooled mixture.

Embodiment M2 is a further embodiment of Embodiment M1, wherein the volume ratio of THF:water in Step (a) is from about 2.9:1 to about 3.1:1.

Embodiment M3 is a further embodiment of Embodiment M1 or M2, wherein the volume ratio of THF:water in Step (a) is about 3.0:1.

Embodiment M4 is a further embodiment of any one of Embodiments M1 to M3, wherein the ratio of the water (volume) to the tris salt of 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid (weight) in Step (a) is about 1.39 mL/g.

Embodiment M5 is a further embodiment of any one of Embodiments M1 to M4, wherein the suspension is heated up at a high temperature of from about 53° C. to about 59° C. (e.g. about 55° C.) to form a solution in Step (b) first, then the temperature of the solution is cooled to a holding temperature of from about 48° C. to about 50° C. while the mixture remains as a solution. In some further embodiments, the suspension is heated up at a high temperature of from about 53° C. to about 57° C. (e.g. about 55° C.) to form a solution in Step (b) first, then the temperature of the solution is cooled to a holding temperature of about 49° C. In some yet further embodiments, the solution in step (b) is mixed at the holding temperature for a period of time greater than about 1 minute, greater than about 5 minutes, greater than about 10 minutes, greater than about 15 minutes, greater than about 20 minutes, greater than about 30 minutes, greater than about 45 minutes, or greater than about 1 hour.

Embodiment M6 is a further embodiment of any one of Embodiments M1 to M5, wherein seeding in Step (c) is carried out so that the temperature of the mixture is maintained without deviating from the holding temperature more than 1.0° C. or more than 2.0° C., and so that at least some seeding material remains as solid after seeding is complete and that some solids exist after the holding period in Step (c). In some yet further embodiments, after seeding the mixture in step (c) is mixed at the holding temperature for a period of time greater than about 1 minute, greater than about 5 minutes, greater than about 10 minutes, greater than about 15 minutes, greater than about 20 minutes, greater than about 30 minutes, greater than about 45 minutes, greater than about 60 minutes, or greater than about 75 minutes.

Embodiment M7 is a further embodiment of any one of Embodiments M1 to M6, wherein the cooling in Step (d) is carried out at a rate of less than about 0.5° C./min, less than about 0.4° C./min, less than about 0.3° C./min, less than about 0.2° C./min (e.g. from about 0.1° C./min to about 0.2° C./min). In some further embodiments, the cooling in Step (d) is carried out at a rate of from about 0.1° C./min to about 0.2° C./min. In some yet further embodiments, after the mixture in step (d) is cooled to the intermediate temperature, the mixture is held at the intermediate temperature for a period of time greater than about 5 minutes, greater than about 10 minutes, greater than about 15 minutes, greater than about 20 minutes, greater than about 30 minutes, greater than about 45 minutes, greater than about 60 minutes, or greater than about 75 minutes.

Embodiment M8 is a further embodiment of any one of Embodiments M1 to M7, wherein the water-miscible organic solvent (e.g. acetonitrile) is added in Step (e) slowly so that the temperature of the mixture is maintained without deviating from the intermediate temperature more than 1.0° C., more than 2.0° C., or more than 3.0° C. In some embodiments, the water-miscible organic solvent is acetonitrile. In some further embodiments, the addition of acetonitrile is carried in a period of time of more than 1 hour, more than 1.5 hours, or more than 2 hours.

Embodiment M9 is a further embodiment of any one of Embodiments M1 to M8, wherein after addition of acetonitrile is complete in Step (e), the resultant mixture is held at the intermediate temperature for a period of time greater than about 1 minute, greater than about 10 minutes, greater than about 15 minutes, greater than about 20 minutes, greater than about 30 minutes, greater than about 45 minutes, greater than about 60 minutes, or greater than about 75 minutes.

Embodiment M10 is a further embodiment of any one of Embodiments M1 to M9, wherein the cooling in Step (f) is carried out at a rate of less than about 0.5° C./min, less than about 0.4° C./min, less than about 0.3° C./min, or less than about 0.2° C./min (e.g. about 0.2° C./min). In some further embodiments, the cooling in Step (f) is carried out at a rate of about 0.2° C./min.

Embodiment M11 is a further embodiment of any one of Embodiments M1 to M10, wherein the holding time in Step (f) is greater than about 1 minute, greater than about 10 minutes, greater than about 15 minutes, greater than about 20 minutes, greater than about 30 minutes, greater than about 45 minutes, greater than about 1 hour, greater than about 2 hours, greater than about 3 hours, greater than about 4 hours, greater than about 5 hours, greater than about 6 hours, greater than about 7 hours, greater than about 8 hours, or greater than about 9 hours. In some further embodiments, the holding time in Step (f) is greater than about 5 hours, about 6 hours, about 7 hours, or about 8 hours.

Embodiment M12 is a further embodiment of any one of Embodiments M1 to M11, wherein after high shear wet milling is performed in Step (h) a sample of the mixture to determine the particle size of the solid in the mixture, if the D90 of the particle size of the solid in the mixture is greater than about 125 μM, then high shear wet milling is performed again until the D90 value is less than about 125 μM.

Embodiment M13 is a further embodiment of any one of Embodiments M1 to M12, wherein isolating the solid in Step (i) comprises filtration, for example, filtration under vacuum.

Embodiment M14 is a further embodiment of Embodiment M13, wherein the filtration or the filtration under vacuum further comprises washing with the solid with methyl ethyl ketone (MEK). In some further embodiments, washing with MEK comprises 1, 2, or 3 washings (e.g. 2 washings). In some yet further embodiments, the amount of the MEK used for each washing is about 2 mL/g to about 2.5 mL/g (e.g. about 2.2 mL/g) based on the amount of the methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate used in Step (a1). In still further embodiments, the MEK used for each washing is at room temperature of optionally cooled to about 10° C. before washing.

Embodiment M15 is a further embodiment of any one of Embodiments M1 to M14, wherein the isolated solid from Step (i) is further dried, for example, dried under vacuum at a temperature of no more than about 40° C., of no more than about 50° C., of no more than about 60° C., or from about 50° C. to about 60° C. (e.g. 55° C.).

### Preparation

C111, tris salt thereof, solid forms of tris salt of C111, and certain intermediates may be prepared by the general and specific methods described below, coupled with the common general knowledge of one skilled in the art of synthetic organic chemistry and/or solid forms of pharmaceutical compounds. Such common general knowledge can be found in standard reference books such as Comprehensive Organic Chemistry, Ed. Barton and Ollis, Elsevier; Comprehensive Organic Transformations: A Guide to Functional Group Preparations, Larock, John Wiley and Sons; and Compendium of Organic Synthetic Methods, Vol. I-XII (published by Wiley-Interscience). The starting materials used herein are commercially available or may be prepared by routine methods known in the art.

In the preparation of the compounds, salts, and solid forms (e.g. crystalline form and amorphous) of the invention, it is noted that some of the preparation methods described herein may require protection of remote functionality (e.g., primary amine, secondary amine, carboxyl in precursors). The need for such protection will vary depending on the nature of the remote functionality and the conditions of the preparation methods. The need for such protection is readily determined by one skilled in the art. The use of such protection/deprotection methods is also within the skill in the art. For a general description of protecting groups and their use, see T. W. Greene, Protective Groups in Organic Synthesis, John Wiley & Sons, New York, 1991.

For example, certain compounds contain primary amines or carboxylic acid functionalities which may interfere with reactions at other sites of the molecule if left unprotected. Accordingly, such functionalities may be protected by an appropriate protecting group which may be removed in a subsequent step. Suitable protecting groups for amine and carboxylic acid protection include those protecting groups commonly used in peptide synthesis (such as N-t-butoxycarbonyl (Boc), benzyloxycarbonyl (Cbz), and 9-fluorenylmethylenoxycarbonyl (Fmoc) for amines and lower alkyl or benzyl esters for carboxylic acids) which are generally not chemically reactive under the reaction conditions described and can typically be removed without chemically altering other functionality in the compounds.

The descriptions below are intended to provide a general description of the methodology employed in the preparation of the compounds and solid forms of the present invention. Some of the compounds of the present invention may contain single or multiple chiral centers with the stereochemical designation (R) or(S). It will be apparent to one skilled in the art that all of the synthetic transformations can be conducted in a similar manner whether the materials are enantioenriched or racemic. Moreover the resolution to the desired optically active material may take place at any desired point in the sequence using well known methods such as described herein and in the chemistry literature. For example, intermediates and finals may be separated using chiral chromatographic methods. Alternatively, chiral salts may be utilized to isolate enantiomerically enriched intermediates and final compounds.

### Examples

The following illustrate the synthesis of non-limiting compounds (including solid forms thereof) of the present invention.

Experiments were generally carried out under inert atmosphere (nitrogen or argon), particularly in cases where oxygen- or moisture-sensitive reagents or intermediates were employed. Commercial solvents and reagents were generally used without further purification. Anhydrous solvents were employed where appropriate, generally AcroSeal® products from Acros Organics, Aldrich® Sure/Seal™ from Sigma-Aldrich, or DriSolv® products from EMD Chemicals. In other cases, commercial solvents were passed through columns packed with 4 Å molecular sieves, until the following QC standards for water were attained: a) <100 ppm for dichloromethane, toluene, N,N-dimethylformamide, and tetrahydrofuran; b) <180 ppm for methanol, ethanol, 1,4-dioxane, and diisopropylamine. For very sensitive reactions, solvents were further treated with metallic sodium, calcium hydride, or molecular sieves, and distilled just prior to use. Products were generally dried under vacuum before being carried on to further reactions or submitted for biological testing. Mass spectrometry data is reported from either liquid chromatography-mass spectrometry (LCMS), atmospheric pressure chemical ionization (APCI) or gas chromatography-mass spectrometry (GCMS) instrumentation. The symbol ♦ denotes that the chlorine isotope pattern was observed in the mass spectrum.

Chiral separations were used to separate enantiomers or diastereomers of some intermediates during the preparation of the compounds of the invention.

Reactions proceeding through detectable intermediates were generally followed by LCMS, and allowed to proceed to full conversion prior to addition of subsequent reagents. For syntheses referencing procedures in other Examples or Methods, reaction conditions (reaction time and temperature) may vary. In general, reactions were followed by thin-layer chromatography or mass spectrometry, and subjected to work-up when appropriate. Purifications may vary between experiments: in general, solvents and the solvent ratios used for eluents/gradients were chosen to provide appropriate Rᵢs or retention times. All starting materials in these Preparations and Examples are either commercially available or can be prepared by methods known in the art or as described herein.

### Example 1. Preparation of 3-Fluoro-4-(((6-(piperidin-4-yl) pyridin-2-yl)oxy)methyl)benzonitrile Bis(4-methylbenzenesulfonate) Salt (anhydrous)

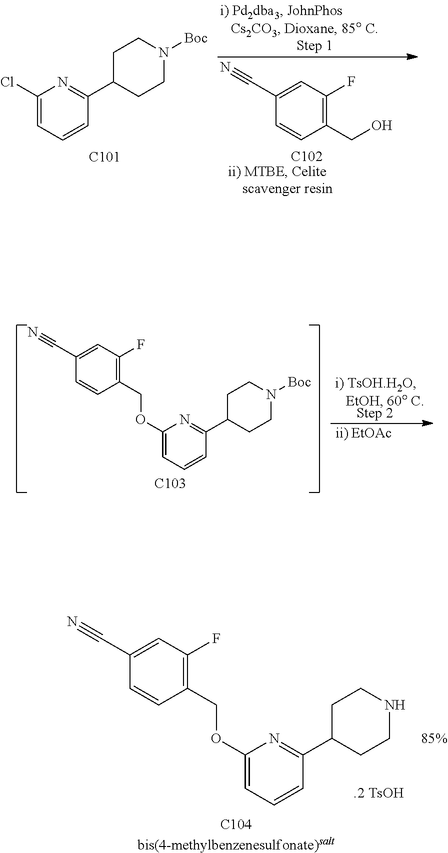

### Step 1. Preparation of C103 [tert-butyl 4-(6-((4-cyano-2-fluorobenzyl)oxy)pyridin-2-yl) piperidine-1-carboxylate]

A clean and dry reactor was evacuated and filled with nitrogen to normal pressure until the oxygen content was ≤1.0%. 1,4-Dioxane (594 kg, 8.3 kg/kg) was charged into the reactor at 15 to 25° C. Maintaining the temperature at 15 to 25° C. C101 [72 kg, 1.0 eq (limiting reagent), tert-butyl 4-(6-chloropyridin-2-yl) piperidine-1-carboxylate] was added into the mixture before C102 (38.5 kg, 1.05 eq., 3-fluoro-4-(hydroxymethyl)benzonitrile) and stirred for 10 to 20 min. Cesium carbonate (132.5 kg, 1.8 kg/kg) was added into the mixture under protection of nitrogen followed by tris (dibenzylideneacetone) dipalladium (2.4 kg, 0.01 eq, Pd₂dba₃) and 2-(Di-tert-butylphosphino) biphenyl (JohnPhos 1.6 kg, 0.02 eq.) The mixture was heated to 83° C. to 90° C. under protection of nitrogen. After 8 to 16 h, upon reaction completion, the reaction mixture was cooled to 15 to 25° C. The mixture was filtered through celite (25 kg, 0.2 kg/kg). The filter cake was washed with MTBE (3×270 kg, 3×3.75 kg/kg, methyl tert-butyl ether). To the combined filtrate at 10 to 35° C. was added a silicon-based metal elimination agent (72 kg, 1 kg/kg). The mixture was agitated for 12 to 18 h at 40 to 50° C. After 12-18 h, upon reaction completion (checked by LC), the mixture was cooled to 20 to 30° C., filtered and the cake was washed with MTBE (324 kg, 4.5 kg/kg). The filtrate was transferred through an in-line filter and concentrated at T≤50° C. under reduced pressure (P≤−0.08 MPa) to a final volume of 2.8 to 3.5 L/kg. Anhydrous ethanol (182 kg, 2.5 kg/kg was added and the mixture concentrated at T≤50° C. under reduced pressure to a final volume of 2.8 to 3.5 L/kg. This process was repeated a further 3 times to provide a concentrated mixture that contained C103.

### Step 2. Preparation of 3-Fluoro-4-(((6-(piperidin-4-yl) pyridin-2-yl)oxy)methyl)benzonitrile bis(4-methylbenzenesulfonate) salt (C104 bis(4-methylbenzenesulfonate))

Anhydrous ethanol (193 kg, 2.7 kg/kg) was added into the concentrated mixture containing C103 from Step 1. In a separate vessel a solution of p-Toluenesulfonic acid (118 kg, 2.55 eq) in anhydrous ethanol (144 kg, 2.0 kg/kg) was prepared at 15 to 25° C. confirming full dissolution. The prepared p-Toluenesulfonic acid/ethanol solution was added to the C103 containing ethanol mixture. The mixture was heated to 55 to 65° C. under the protection of nitrogen. After 2 to 4 h, the mixture was sampled for HPLC analysis. Maintaining the temperature at 55 to 65° C., ethyl acetate (580 kg, 8 kg/kg) was added into the mixture. The mixture was cooled to 0 to 5° C. resulting in crystallization. The mixture was slurried at 0 to 5° C. for 2-5 h, and then was filtered. The filter cake was washed with ethyl acetate (2×90 kg, 1.25 kg/kg). The solid was dried at 30 to 40° C. for 8 to 16 h. The solid was cooled to 15 to 30° C. before collection (weight 135 kg). Karl Fischer titration was carried out to determine the solid collected to be anhydrous.

¹H NMR (600 MHz, DMSO-d6) δ: 8.53 (br s, 1H), 8.26 (br s, 1H), 7.89 (d, 1H), 7.67-7.78 (m, 3H), 7.48 (d, 4H), 7.11 (d, 4H), 6.90 (d, 1H), 6.79 (d, 1H), 5.48 (s, 2H), 3.35 (d, 2H), 2.96-3.09 (m, 2H), 2.79-2.96 (m, 1H), 2.29 (s, 6H), 1.93-2.03 (m, 2H), 1.77-1.90 (m, 2H).

The relative amount of each reagent used in this example refers to the limiting reagent, which is C101.

### Example 2. Alternative Preparation of 3-Fluoro-4-(((6-(piperidin-4-yl) pyridin-2-yl)oxy)methyl)benzonitrile Bis(4-methylbenzenesulfonate) Salt (anhydrous)

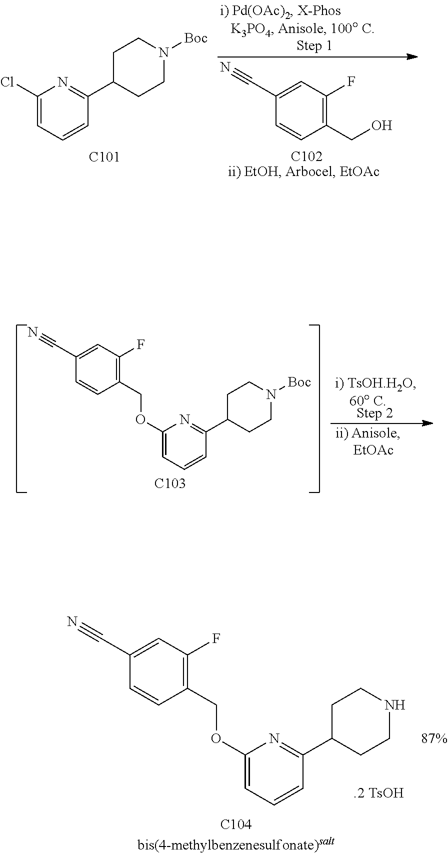

### Step 1. Preparation of C103 [tert-butyl 4-(6-((4-cyano-2-fluorobenzyl)oxy)pyridin-2-yl) piperidine-1-carboxylate]

To a clean reactor vessel was charged C101 (25 g, 1.0 eq., tert-butyl 4-(6-chloropyridin-2-yl) piperidine-1-carboxylate, limiting reagent), C102 (13.5 g, 1.05 eq., 3-fluoro-4-(hydroxymethyl)benzonitrile) and Potassium Phosphate Tribasic (31.1 g, 1.7 eq.). To the reactor was charged Anisole (200 ml, 8 ml/g) in one portion and the reactor stirred at under a nitrogen sweep at 25° C. to degas the system (Target<0.15% w/w O₂). In a separate vessel was dissolved Palladium (II) acetate (0.05 g, 0.0025 eq.) in Anisole (1.0 ml, 0.04 ml/g) at 20° C. to give a clear pale orange/brown solution. In a separate vessel was dissolved X-Phos (0.1 g, 0.0025 eq.) in Anisole (1.0 ml, 0.04 ml/g) to give a clear solution. The solutions of Palladium acetate and X-Phos were charged to the reactor vessel via syringe in one portion. The resulting suspension was stirred at 100° C. for 20 hours and then sampled to check for reaction completion (LC). Upon reaction completion the reaction mixture was cooled to 20-25° C. and to the clear orange solution was added Ethanol (50 ml, 2 mL/g) at 20-25° C. before being filtered through Arbocel® (˜6.25 g, 0.25 g/g). The Arbocel® filter cake was washed with Ethyl Acetate (100 ml, 4 ml/g).

### Step 2. Preparation of 3-Fluoro-4-(((6-(piperidin-4-yl) pyridin-2-yl)oxy)methyl)benzonitrile bis(4-methylbenzenesulfonate) salt (C104 bis(4-methylbenzenesulfonate))

The resulting filtrate from Step 1 was charged to a clean reactor vessel. p-Toluenesulfonic acid monohydrate (36 g, 2.2 eq) was added in one portion at 20-25° C. and the suspension stirred until full dissolution. The reaction mixture was stirred at 40° C. for 20 hours (overnight) and then sampled for reaction completion. Upon reaction completion the pale yellow suspension was cooled to 0° C. and stirred for at least 1 hour. The suspension was filtered under vacuum with Anisole (100 ml, 4 ml/g) and used as a cake wash in 2 portions (50 ml, 2 ml/g per portion). The filter cake was further washed with Ethyl Acetate (50 ml, 2 ml/g) before transferring to vacuum oven. The product was dried under vacuum for 16 hours (overnight) at 50° C. before an additional 16 hours (overnight) at 60° C. Crude C104 bis(4-methylbenzenesulfonate) salt was isolated as a white-off white solid (48.3 g, 87% yield). Karl Fischer titration was carried out to determine the white-off white solid to be anhydrous.

The relative amount of each reagent used in this example refers to the limiting reagent, which is C101.

Powder X-Ray Diffraction (PXRD) Method (Used Herein for all the PXRD Data Described in this Patent).

A powder x-ray diffraction pattern was generated using a Bruker AXS D8 Endeavor diffractometer equipped with a Cu radiation source. The tube voltage and amperage were set to 40 kV and 40 mA, respectively. The motorized divergence slits were set at constant illumination of 11 mm. Diffracted radiation was detected using a LYNXEYE XE-T energy dispersive X-ray detector, with the position sensitive detector (PSD) opening set at 4.00°. Data was collected on the theta-theta goniometer at the Cu wavelength from 2.0 to 55.0 degrees 2-theta (° 2θ) using a step size of 0.019° 2θ and a time per step of 0.2 seconds. Samples were prepared for analysis by placing them in a silicon low background small divot holder and rotated at 15 rpm during data collection. Data were analyzed in DIFFRAC.EVA V5.0 software. Peak lists were prepared using reflections with a relative intensity≥5% of the most intense band in each respective diffraction pattern. A typical error of ±0.2° 2θ in peak positions (USP-941) applies to this data. The minor error associated with this measurement can occur because of a variety of factors including: (a) sample preparation (e.g., sample height), (b) instrument characteristics, (c) instrument calibration, (d) operator input (e.g. in determining the peak locations), and (e) the nature of the material (e.g. preferred orientation and transparency effects).

A sample of the anhydrous bis-tosylate salt of C104 (as prepared by a procedure either as Example 1 or 2) was used to generate the powder X-ray diffraction pattern (herein designated as Form 1). The PXRD pattern for Form 1 of bis-tosylate salt of C104 is provided in FIG. 1 and the corresponding peak list is provided in Table E2 (with peaks the relative intensity of which is 5% or greater).

TABLE E2
PXRD Peak Table for bis-tosylate
salt of C104 (Anhydrous Form 1)

| Angle, | Rel. |
|---|---|
| °2Theta (°2θ) | Intensity, % |
| 5.0 | 100.0 |
| 7.5 | 37.0 |
| 10.0 | 13.3 |
| 11.9 | 16.0 |
| 12.8 | 14.7 |
| 13.3 | 26.6 |
| 14.7 | 43.5 |
| 14.9 | 8.0 |
| 15.5 | 24.1 |
| 17.2 | 12.4 |
| 17.4 | 41.4 |
| 18.1 | 5.3 |
| 19.1 | 18.7 |
| 19.9 | 16.9 |
| 21.1 | 11.7 |
| 21.7 | 5.0 |
| 22.1 | 14.1 |
| 23.8 | 52.8 |
| 24.2 | 13.7 |
| 24.5 | 9.2 |
| 25.0 | 18.2 |
| 25.6 | 9.2 |
| 27.7 | 7.8 |
| 28.1 | 18.6 |
| 28.4 | 13.0 |
| 28.9 | 8.7 |

### Example 3. Preparation of 3-Fluoro-4-(((6-(piperidin-4-yl) pyridin-2-yl)oxy)methyl)benzonitrile Bis(4-methylbenzenesulfonate) Salt (mono hydrate)

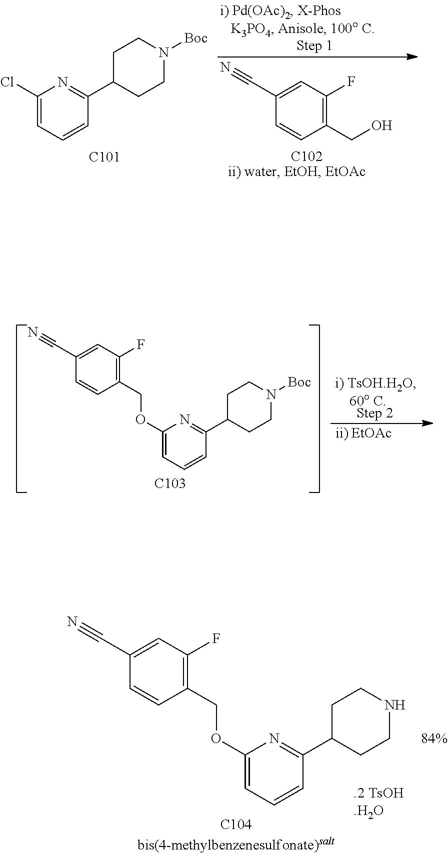

### Step 1. Preparation of C103 [tert-butyl 4-(6-((4-cyano-2-fluorobenzyl)oxy)pyridin-2-yl) piperidine-1-carboxylate]

To a clean reactor vessel was charged C101 [25 g, 1.0 eq., limiting reagent, tert-butyl 4-(6-chloropyridin-2-yl) piperidine-1-carboxylate], C102 (13.5 g, 1.05 eq., 3-fluoro-4-(hydroxymethyl)benzonitrile), and Potassium Phosphate Tribasic (31.1 g, 1.7 eq.). To the reactor was charged anisole (200 ml, 8 ml/g) in one portion and the reactor stirred at under a nitrogen sweep at 25° C. to degas the system (Target<0.15% w/w O₂). In a separate vessel was dissolved Palladium (II) acetate (0.05 g, 0.0025 eq.) in anisole (5.0 ml, 0.2 ml/g) at 20° C. to give a clear pale orange/brown solution. In a separate vessel was dissolved X-Phos (0.1 g, 0.0025 eq.) in anisole (5.0 ml, 0.2 ml/g) to give a clear solution. The solutions of Palladium acetate and X-Phos were charged to the reactor vessel via syringe in one portion. The resulting suspension was stirred at 100° C. for 16 hours and then sampled to check for reaction completion (LC). Upon reaction completion the reaction mixture was cooled to 20-25° C. and to the reaction mixture was added water (75 ml, 3 ml/g), ethyl acetate (100 ml, 4 ml/g) and ethanol (50 ml, 2 ml/g). The layers were allowed to settle, and the phases were separated.

### Step 2. Preparation of 3-Fluoro-4-(((6-(piperidin-4-yl) pyridin-2-yl)oxy)methyl)benzonitrile bis(4-methylbenzenesulfonate) salt (C104 bis(4-methylbenzenesulfonate))

To the organic phase (from Step 1) was added p-Toluenesulfonic acid monohydrate (40.7 g, 2.5 eq) was added in one portion at 20-25° C. and the reaction mixture was heated to 40° C. After 20 hours the slurry was sampled to check for reaction completion. Upon reaction completion the suspension was cooled to 5° C. and stirred for at least 1 hour. The suspension was filtered under vacuum and the filter cake was washed twice with Ethyl Acetate (2×50 ml, 2×2 ml/g) before transferring to vacuum oven. The product was dried under vacuum for 16 hours (overnight) at 50° C. before an additional 16 hours (overnight) at 60° C. Crude C104 bis(4-methylbenzenesulfonate) salt was isolated as a white-off white solid (46.4 g, 84% yield). Karl Fischer titration was carried out to determine the white-off white solid to be a monohydrate.

The relative amount of each reagent used in this example refers to the limiting reagent, which is C101.

| | |
|---|---|
| Salt Stoichiometry | C104:TsOH = 1.0:2.0 |
| ¹H NMR | ¹H NMR (600 MHz, DMSO-d₆) δ = 8.57 (br s, 1H), 8.30 (br s, 1H), |
| | 7.90 (dd, J = 1.1, 9.9 Hz, 1H), 7.75-7.73 (m, 1H), 7.73-7.72 (m, |
| | 1H), 7.69 (dd, J = 7.2, 8.2 Hz, 1H), 7.53-7.42 (m, 4H), 7.18-7.05 |
| | (m, 4H), 6.89 (d, J = 7.2 Hz, 1H), 6.78 (d, J = 8.2 Hz, 1H), 5.47 (s, |
| | 2H), 3.43-3.30 (m, 2H), 3.00 (ddddd, J = 3.0 (x2), 10.8, 12.8 (x2) |
| | Hz, 2H), 2.91 (dddd, J = 3.7, 11.7 Hz, 1H), 2.28 (s, 6H), 2.02-1.90 |
| | (m, 2H), 1.83 (dddd, J = 4.2, 11.7, 12.9, 14.2 Hz, 2H) |
| ¹³C NMR | ¹³C {1H} NMR (151 MHz, DMSO-d₆) δ = 161.76, 160.57, 159.59 |
| | (d, J = 248.5 Hz, 1C), 145.39 (s, 2C), 140.24, 137.81 (s, 2C), |
| | 131.33 (d, J = 4.6 Hz, 1C), 130.72 (d, J = 14.4 Hz, 1C), 128.82 (d, |
| | J = 4.0 Hz, 1C), 128.12 (s, 4C), 125.46 (s, 4C), 119.29 (d, J = 25.4 |
| | Hz, 1C), 117.59 (d, J = 2.9 Hz, 1C), 114.77, 112.12 (d, J = 10.4 Hz, |
| | 1C), 108.72, 60.18 (d, J = 3.5 Hz, 1C), 43.21 (s, 2C), 39.79, 27.71 |
| | (s, 2C), 20.79 (s, 2C) |
| MS | Measured m/z 312.1506, calculated m/z 312.1507 |

A sample of the monohydrate bis-tosylate salt of C104 (as prepared by a procedure as described in Example 3) was used to generate the powder X-ray diffraction pattern (herein designated as Form 2). The PXRD pattern for Form 2 of bis-tosylate salt of C104 (monohydrate) is provided in FIG. 2 and the corresponding peak list is provided in Table E3 (with peaks the relative intensity of which is 5% or greater).

TABLE E3
PXRD Peak Table for bis-tosylate
salt of C104 Monohydrate (Form 2)

| Angle, °2Theta (°2θ) | Rel. Intensity, % |
|---|---|
| 5.0 | 100.0 |
| 7.5 | 26.7 |
| 9.9 | 8.6 |
| 12.4 | 34.0 |
| 13.0 | 17.1 |
| 13.7 | 12.6 |
| 15.3 | 9.8 |
| 16.6 | 31.8 |
| 17.0 | 28.1 |
| 17.4 | 31.3 |
| 18.3 | 24.4 |
| 19.0 | 18.5 |
| 19.5 | 7.1 |
| 19.8 | 9.8 |
| 19.9 | 17.8 |
| 20.6 | 6.0 |
| 21.2 | 37.1 |
| 21.5 | 6.1 |
| 21.7 | 5.2 |
| 22.3 | 20.5 |
| 22.5 | 46.0 |
| 22.7 | 67.0 |
| 23.8 | 11.6 |
| 23.9 | 16.0 |
| 25.0 | 10.0 |
| 25.6 | 7.6 |
| 27.9 | 41.5 |
| 28.0 | 6.9 |
| 28.5 | 5.1 |
| 29.5 | 6.6 |

### Example 4. A Yet Further Alternative Preparation of 3-Fluoro-4-(((6-(piperidin-4-yl) pyridin-2-yl)oxy)methyl)benzonitrile Bis(4-methylbenzenesulfonate) Salt (anhydrous)

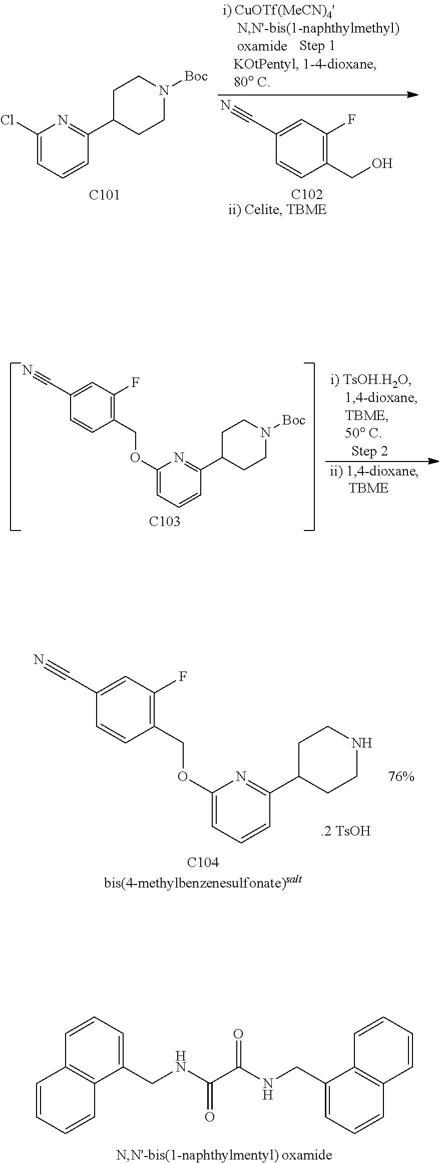

### Step 1. Preparation of C103 [tert-butyl 4-(6-((4-cyano-2-fluorobenzyl)oxy)pyridin-2-yl) piperidine-1-carboxylate]

C101 (3.00 g, limiting reagent), C102 (1.83 g, 1.2 eq.), N,N′-bis(1-naphthylmethyl) oxamide (0.219 g, 0.05 eq.), tetrakisacetonitrile copper (I) triflate (0.194 g, 0.05 eq.) Phenanthrene (0.139 ml, 0.08 eq.) and 1,4-dioxane (24 mL, 8 ml/g) charged to a reaction vessel in glovebox at <100 ppm O₂ (actual=66 ppm). Sodium tert-pentoxide (in toluene) (4.2 mL, 3.6 mol/L, 1.5 eq.) was added in one portion and the resultant reaction mixture heated to 80° C. Upon reaction completion the reaction mixture was cooled to 20-25° C. and filtered through celite. The reaction vessel was rinsed with 1,4-dioxane (5 ml, 1.67 ml/g), the celite filter cake was washed with methyl tert-butyl ether (MTBE, 2×10 ml, 2×3.33 ml/g) and the combined organics concentrated in vacuo to provide an oil.

### Step 2. Preparation of 3-Fluoro-4-(((6-(piperidin-4-yl) pyridin-2-yl)oxy)methyl)benzonitrile bis(4-methylbenzenesulfonate) salt [C104 bis(4-methylbenzenesulfonate)]

The resulting oil from Step 1 was redissolved in 1,4-dioxane (5 ml, 1.67 ml/g) and MTBE (2.5 ml, 0.83 ml/g); and p-toluenesulfonic acid monohydrate (4.1 g, 2.1 eq.) was added, then the resultant mixture was heated to 50° C. Additional 1,4-dioxane (10 ml, 3.33 ml/g) was added to mobilize the slurry. Solids were isolated by filtration and washed with 1:1 1,4-dioxane:MTBE (10 ml, 3.33 ml/g) before drying in vac oven at 60° C. to afford C104 bis(4-methylbenzenesulfonate) salt (5.05 g, 76.2% yield). Karl Fischer titration was carried out to determine the dried final product to be anhydrous.

The relative amount of each reagent used in this example compares to the limiting reagent, which is C101.

### Example 5. Preparation of Mono Tosylate Salt, Bis Mesylate Salt, Mono Mesylate Salt, Mono Sulfate Salt, and Hemi Sulfate Salt of C104

General procedure for the preparation of Mono Tosylate Salt, Bis Mesylate Salt, Mono Mesylate Salt, Mono Sulfate Salt, and Hemi Sulfate Salt of C104:

C104 (250 mg, 1.0 eq. limiting reagent) was dissolved with stirring in Anisole (2 ml, 8 ml/g). To the solution was added the desired acid in defined stoichiometry (0.5 eq-2.0 eq). To the mixture was then added ethyl acetate (2 ml, 8 ml/g) as antisolvent. The resulting slurries were filtered and dried in a vacuum oven at 50° C.

Analytical data for the salts prepared were collected.

### 5A. Mono Tosylate Salt of C104

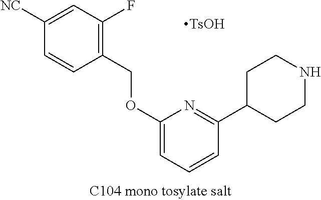

| | |
|---|---|
| Salt Stoichiometry | C104:TsOH (p-Toluenesulfonic acid) = 1.0:1.0 |
| ¹H NMR | ¹H NMR (600 MHz, DMSO-d₆) δ = 8.41 (br s, 2H), 7.90 (dd, J = 1.4, |
| | 10.4 Hz, 1H), 7.76-7.71 (m, 2H), 7.70 (dd, J = 7.2, 7.9 Hz, 1H), 7.53- |
| | 7.44 (m, 2H), 7.17-7.06 (m, 2H), 6.90 (d, J = 7.2 Hz, 1H), 6.78 (d, J = |
| | 7.9 Hz, 1H), 5.48 (s, 2H), 3.40-3.30 (m, 2H), 3.01 (dt, J = 3.0, 12.8 |
| | Hz, 2H), 2.91 (tt, J = 3.7, 11.6 Hz, 1H), 2.28 (s, 3H), 2.00-1.93 (m, |
| | 2H), 1.83 (dddd, J = 3.0, 11.6, 12.8, 14.1 Hz, 2H) |
| ¹³C NMR | ¹³C {1H} NMR (151 MHz, DMSO-d₆) δ = 161.77, 160.57, 159.58 (d, |
| | J = 248.8 Hz, 1C), 145.57, 140.20, 137.68, 131.29 (d, J = 4.9 Hz, 1C), |
| | 130.72 (d, J = 14.7 Hz, 1C), 128.81 (d, J = 3.8 Hz, 1C), 128.09, 125.47, |
| | 119.28 (d, J = 25.4 Hz, 1C), 117.58 (d, J = 2.6 Hz, 1C), 114.77, 112.11 |
| | (d, J = 9.8 Hz, 1C), 108.71, 60.15 (d, J = 3.5 Hz, 1C), 43.20 (s, 2C), |
| | 39.93, 27.71 (s, 2C), 20.76 |
| MS | Measured m/z 312.1507, calculated m/z 312.1507 |

A sample of the Mono Tosylate (toluenesulfonate) Salt of C104 (as prepared by a procedure as described in Example 5) was used to generate the powder X-ray diffraction pattern. The PXRD pattern for the Mono Tosylate Salt of C104 (as prepared by a procedure as described in Example 5) is provided in FIG. 3.

### 5B. Bis Mesylate Salt of C104

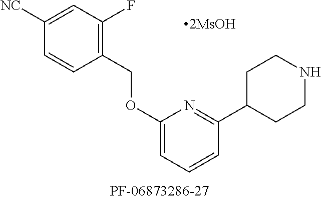 [SMILES: N#Cc1ccc(COc2cccc(C3CCNCC3)n2)c(F)c1 ; "·2MsOH"]

| | |
|---|---|
| Salt Stoichiometry | C104:MsOH (Methanesulfonic acid) = 1.0:2.0 |
| ¹H NMR | ¹H NMR (400 MHz, DMSO-d₆) δ = 8.60 (br s, 1H), 8.33 (br s, 1H), |
| | 7.88 (dd, J = 1.6, 9.9 Hz, 1H), 7.81-7.63 (m, 3H), 6.91 (d, J = 7.2 |
| | Hz, 1H), 6.78 (d, J = 8.0 Hz, 1H), 5.48 (s, 2H), 3.42-3.31 (m, 2H), |
| | 3.01 (tq, J = 3.6, 12.8 Hz, 2H), 2.92 (tt, J = 3.6, 11.7 Hz, 1H), 2.41 |
| | (s, 6H), 2.06-1.92 (m, 2H), 1.85 (dddd, J = 3.6, 11.7, 12.8, 13.5 |
| | Hz, 2H) |
| ¹³C NMR | ¹³C {¹H} NMR (101 MHz, DMSO-d₆) δ = 161.75, 160.55, 159.70 |
| | (d, J = 224.0 Hz, 1C), 140.21, 131.30 (d, J = 4.7 Hz, 1C), 130.58 |
| | (d, J = 15.0 Hz, 1C), 128.79 (d, J = 3.5 Hz, 1C), 119.25 (d, J = 23.0 |
| | Hz, 1C), 117.52 (d, J = 3.0 Hz, 1C), 114.74, 112.12 (d, J = 11.0 |
| | Hz, 1C), 108.68, 60.13 (d, J = 4.0 Hz, 1C), 43.16 (s, 2C), 39.97, |
| | 39.67 (s, 2C), 27.67 (s, 2C) |
| MS | Measured m/z 312.1515, calculated m/z 312.1507 |

A sample of the Bis Mesylate (methanesulfonate) Salt of C104 (as prepared by a procedure as described in Example 5) was used to generate the powder X-ray diffraction pattern. The PXRD pattern for the Bis Mesylate Salt of C104 (as prepared by a procedure as described in Example 5) is provided in FIG. 4.

### 5C. Mono Mesylate Salt of C104

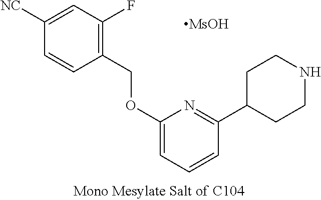 [SMILES: "·MsOH" ; N#Cc1ccc(COc2cccc(C3CCNCC3)n2)c(F)c1]

| | |
|---|---|
| Salt Stoichiometry | C104:MsOH = 1.0:1.2 |
| ¹H NMR | ¹H NMR (600 MHz, DMSO-d₆) δ = 8.62 (br s, 1H), 8.35 (br s, 1H), 7.90 (dd, J = |
| | 1.4, 10.2 Hz, 1H), 7.76-7.72 (m, 2H), 7.70 (dd, J = 7.2, 7.8 Hz, 1H), 6.91 (d, J = |
| | 7.2 Hz, 1H), 6.78 (d, J = 7.8 Hz, 1H), 5.48 (s, 2H), 3.38-3.32 (m, 2H), 3.01 (tdt, |
| | J = 3.4, 11.0, 12.8 Hz, 2H), 2.91 (tt, J = 3.7, 11.6 Hz, 1H), 2.36 (s, 3H), 1.99- |
| | 1.92 (m, 2H), 1.85 (dddd, J = 3.4, 11.6, 12.8, 13.8 Hz, 2H) |
| ¹³C NMR | ¹³C {1H} NMR (151 MHz, DMSO-d₆) δ = 161.77, 160.60, 159.60 (d, J = 248.7 |
| | Hz, 1C), 140.23, 131.34 (d, J = 5.0 Hz, 1C), 130.73 (d, J = 14.6 Hz, 1C), 128.83 |
| | (d, J = 3.6 Hz, 1C), 119.29 (d, J = 25.3 Hz, 1C), 117.59 (d, J = 2.5 Hz, 1C), |
| | 114.77, 112.11 (d, J = 9.9 Hz, 1C), 108.71, 60.17 (d, J = 3.6 Hz, 1C), 43.16 (s, |
| | 2C), 39.97, 39.74, 27.69 (s, 2C) |
| MS | Measured m/z 312.1505, calculated m/z 312.1507, difference = 0.2 mDa |

A sample of the Mono Mesylate Salt of C104 (as prepared by a procedure as described in Example 5) was used to generate the powder X-ray diffraction pattern. The PXRD pattern for the Mono Mesylate Salt of C104 (as prepared by a procedure as described in Example 5) is provided in FIG. 5.

### 5D. Mono Sulfate Salt of C104

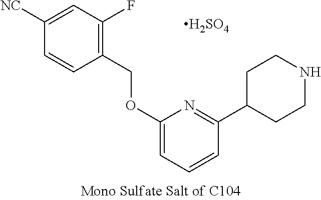

| Characterisation | Results |
|---|---|
| Salt Stoichiometry | C104:H₂SO₄ = 1.0:1.3 (measure), 1:0:1.0 (theoretical) |
| ¹H NMR | ¹H NMR (600 MHz, ACETONITRILE-d₃) δ = 7.71 (dd, J = 7.4, 7.9 Hz, 1H), |
| | 7.67 (dd, J = 7.4, 7.8 Hz, 1H), 7.56 (dd, J = 1.5, 7.8 Hz, 1H), 7.55 (dd, J = |
| | 1.5, 9.8 Hz, 1H), 6.92 (d, J = 7.4 Hz, 1H), 6.77 (d, J = 7.9 Hz, 1H), 5.47 (s, |
| | 2H), 3.42 (ddd, J = 2.8, 12.9 Hz, 2H), 3.03 (ddd, J = 2.8, 12.9 Hz, 2H), 2.92 |
| | (dddd, J = 3.7, 11.6 Hz, 1H), 1.99-2.06 (m, 2H), 1.90 (dddd, J = 4.2, 11.6, |
| | 12.9, 14.0 Hz, 2H) |
| ¹³C NMR | ¹³C NMR {¹H} (151 MHz, ACETONITRILE-d₃) δ = 163.07, 161.15, 160.99 |
| | (d, J = 249.1 Hz, 1C), 142.27, 132.21 (d, J = 4.6 Hz, 1C), 131.84 (d, J = |
| | 14.7 Hz, 1C), 120.27 (d, J = 25.4 Hz, 1C), 118.66 (d, J = 2.9 Hz, 1C), |
| | 116.16, 113.61 (d, J = 9.8 Hz, 1C), 109.74, 62.21 (d, J = 4.0 Hz, 1C), 44.77 |
| | (s, 2C), 40.99, 28.62 (s, 2C), 1.52 |
| MS | Measured m/z 312.1515, calculated m/z 312.1507 |

A sample of the Mono Sulfate Salt of C104 (as prepared by a procedure as described in Example 5) was used to generate the powder X-ray diffraction pattern. The PXRD pattern for the Mono Sulfate Salt of C104 (as prepared by a procedure as described in Example 5) is provided in FIG. 6.

### 5E. Hemi Sulfate Salt of C104

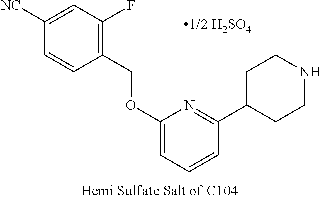 [SMILES: N#Cc1ccc(COc2cccc(C3CCNCC3)n2)c(F)c1 ; "·1/2 H2SO4"]

| | |
|---|---|
| Salt Stoichiometry | C104:H₂SO₄ = 1.0:0.5 |
| ¹H NMR | ¹H NMR (600 MHz, ACETONITRILE-d₃) δ = 7.66 (t, J = 7.8 Hz, |
| | 1H), 7.62 (dd, J = 7.4, 8.2 Hz, 1H), 7.54 (dd, J = 1.5, 7.8 Hz, 1H), |
| | 7.53 (dd, J = 1.5, 9.8 Hz, 1H), 6.87 (d, J = 7.4 Hz, 1H), 6.68 (d, J = |
| | 8.3 Hz, 1H), 5.44 (s, 2H), 3.45-3.40 (m, 2H), 3.01 (dt, J = 3.5, 13.0 |
| | Hz, 2H), 2.87 (tt, J = 3.7, 11.6 Hz, 1H), 2.04-1.95 (m, 2H), 1.88 |
| | (dddd, J = 3.6, 11.6, 13.0, 14.4 Hz, 2H) |
| ¹³C NMR | ¹³C {1H} NMR (151 MHz, ACETONITRILE-d₃) δ = 163.26, 161.74, |
| | 161.00 (d, J = 249.0 Hz, 1C), 141.34, 132.24 (d, J = 4.7 Hz, 1C), |
| | 132.18 (d, J = 14.3 Hz, 1C), 129.72 (d, J = 3.6 Hz, 1C), 120.23 (d, |
| | J = 25.6 Hz, 1C), 118.73 (d, J = 3.0 Hz, 1C), 115.96, 113.43 (d, J = |
| | 9.9 Hz, 1C), 109.56, 61.71 (d, J = 4.1 Hz, 1C), 44.76 (s, 2C), 41.39, |
| | 28.73 (s, 2C) |
| MS | Measured m/z 312.1515, calculated m/z 312.1507 |

A sample of the Hemi Sulfate Salt of C104 (as prepared by a procedure as described in Example 5) was used to generate the powder X-ray diffraction pattern. The PXRD pattern for the Hemi Sulfate Salt of C104 (as prepared by a procedure as described in Example 5) is provided in FIG. 7.

### Example 6. Preparation of Methyl(S)-2-(chloromethyl)-1-(oxetan-2-ylmethyl)-1H-benzo[d]imidazole-6-carboxylate (C109)

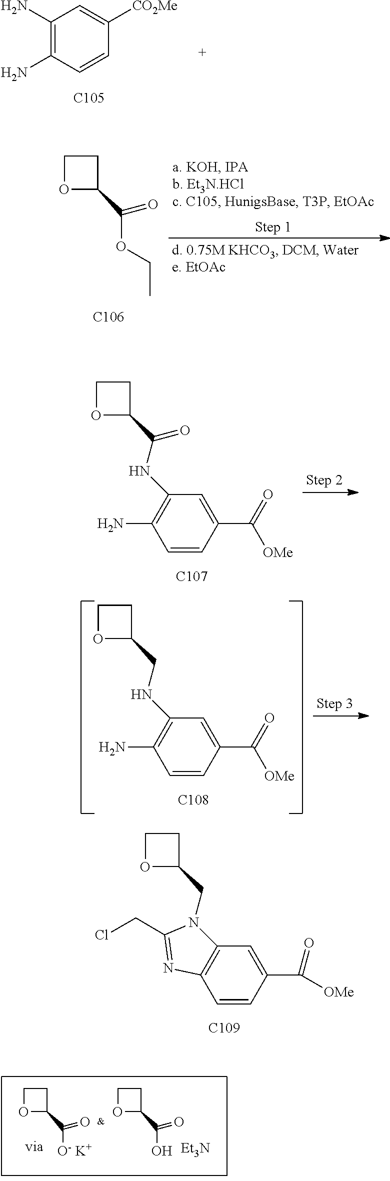

### Step 1. Preparation of methyl(S)-4-amino-3-(oxetane-2-carboxamido)benzoate (C107)

To a Jacketed vessel with overhead stirring and nitrogen purged was added Isopropanol (4.0 mL/g, 15.7 g, 20.0 mL) followed by Potassium Hydroxide Pellets (85 mass %, 1.65 equiv., 3.28 g, 49.6 mmol). The mixture was heated to 40° C. for 30 minutes to dissolve most/all the KOH. Then the mixture was Cooled to 10° C. at 0.5° C./minute. C106 (88.4 mass %, 1.05 equiv., 4.65 g, 31.6 mmol; ethyl(S)-oxetane-2-carboxylate) was then added over 15-30 mins. Once the addition was complete the reaction mixture was stirred for 30 minutes at 10° C. and then warmed to 25° C. at 0.5° C./minute, and the reaction mixture was Left to stir at 25° C. After 1 hour, the reaction mixture was sampled for reaction completion (ester hydrolysis, which was checked by ¹H NMR in CD₃OD). To the reaction mixture at 25° C. was added Triethylamine Hydrochloride (1.95 equiv., 8.08 g, 58.7 mmol), and the resultant mixture was left to stir at 25° C. After ˜ 12 hours at 25° C. the reaction mixture was diluted with Ethyl Acetate (for reaction) (10.0 mL/g, 45.0 g, 50.0 mL). To the reaction mixture was then added C105 (5.0 g, 30.1 mmol; methyl 3,4-diaminobenzoate, limiting reagent). To the reaction mixture was then added Diisopropylethylamine (3.00 equiv., 11.7 g, 15.7 mL, 90.3 mmol). The reaction mixture was then stirred at 25° C. for 30 minutes to homogenize/dissolve C105 before cooling to 10° C. at 0.5° C./minute. To the reaction mixture was then added 50 wt % T3P (propanephosphonic acid anhydride, aka. PPAA, cyclic trimer) in EtOAc (ethyl acetate) (50 mass %, 1.50 equiv., 28.7 g, 26.9 mL, 45.1 mmol) over 30 minutes maintaining temperature at 8-15° C. The resultant reaction mixture was left to stir at 10° C. After 1 Hour the reaction mixture was sampled for reaction completion (HPLC analysis for in-process check). The reaction mixture was quenched with Potassium Hydrogen Carbonate (aq.) (0.75M) (10.0 mL/g, 50.0 mL). The reaction mixture was warmed to 25° C. and stirred to fully mix the phases before stirring stopped and phases allowed to separate (two clear phases, no solids). The lower aqueous phase was drained from the vessel. The aqueous phase was then back extracted with Dichloromethane (10.0 mL/g, 66.3 g, 50.0 mL). Stirred to fully mix the phases before stirring stopped and phases allowed to separate (two clear phases, no solids). The phases were separated, and upper aqueous phase sent to waste. The EtOAc & DCM extracts were combined. The combined organic phases were then washed with Water (3.0 mL/g, 15.0 g, 15.0 mL). The phases were separated, and upper aqueous phase sent to waste. The product solution was concentrated to (˜12 mL/g, 60 mL) at jacket temp 40-50° C. and 150-250 mbar. Vacuum was released and the mixture self-seeded. Stirred at 40-45° C. for 15 minutes as material started to crystallize. The product solution was concentrated to (˜8 mL/g, 40 mL) at jacket temp 40-50° C. and 150-250 mbar. Vacuum was released and the resulting fine suspension was held at 40-45° C. for 30 mins before cooling to 5° C. at 0.2° C./minute. The suspension was stirred at 5° C. for 1 hour. The suspension was filtered using vacuum filtration. The filter cake was washed with pre-cooled Ethyl Acetate (EtOAc, 2.0 mL/g, 9.00 g, 10.0 mL). After pulling dry for 1 hour the solid was dried at 40-50° C. under vacuum for 12 to 48 hours. On removing from the oven C107 was obtained as an off-white solid (Crude Weight: 4.62 g, Crude Yield: 61%).

| | |
|---|---|
| ¹H NMR | ¹H NMR (400 MHz, DMSO-d₆) δ: 9.24 (br s, 1H), 7.83 (d, J = 2.0 Hz, 1H), |
| | 7.57 (dd, J = 8.5, 2.0 Hz, 1H), 6.76 (d, J = 8.5 Hz, 1H), 5.73 (s, 2H), 5.10 (dd, |
| | J = 8.8, 6.5 Hz, 1H), 4.55-4.76 (m, 2H), 3.76 (s, 3H), 2.96 (dddd, J = 11.2, |
| | 8.8(x2), 6.5 Hz, 1H), 2.66 (dddd, J = 11.2, 8.8, 6.5(x2) Hz, 1H) |
| ¹³C NMR | ¹³C {1H} NMR (101 MHz, DMSO-d₆) δ: 171.3, 166.0, 147.7, 128.2, 128.0, |
| | 121.1, 116.3, 114.7, 78.8, 68.9, 51.3, 25.6 |
| MS | Measured m/z 251.1024, calculated m/z 251.1026 |

The relative amount of each reagent used in Step 1 of this example compares to the limiting reagent, which is C105.

### Step 2. Preparation of Methyl(S)-4-amino-3-((oxetan-2-ylmethyl)amino)benzoate (C108)

To a Jacketed vessel with overhead stirring and nitrogen purged was added Dichloromethane (DCM, 15.0 mL/g, 1590 g, 1200 mL). To the reactor vessel was added C107 (80.00 g, 319.7 mmol; limiting reagent; methyl(S)-4-amino-3-(oxetane-2-carboxamido)benzoate) and stirred at 20° C. To the reaction was then added triethyl borate (3.00 eq., 140 g, 164 mL, 959 mmol) and stirred at 20° C. To the suspension was added lithium borohydride (2 M) in tetrahydrofuran (1.25 equiv., 180 g, 200 mL, 400 mmol) over 30 mins and stirred at 20° C. Once the addition of LiBH₄ was complete the reaction was left to stir at 20° C. and sampled periodically to check for reaction completion. Upon reaction completion the reaction mixture was slowly quenched with water (5.0 mL/g, 400.0 g, 400.0 mL) over no less than 30 mins. The resulting mixture was stirred for no less than 2 hours before proceeding to the phase separation. Stirring stopped and phases were allowed to separate, the lower DCM phase was drained off and transferred to a holding vessel. To the DCM product solution was then added phosphoric Acid (0.5M) in water (5.0 mL/g, 400.0 mL) and stirred at 20° C. for 30 mins. Stirring stopped and phases allowed to separate, the lower DCM phase was drained off and transferred to a holding vessel. To the DCM product solution was then added aqueous citric acid (0.5M) in water (5.0 mL/g, 400.0 mL) and stirred at 20° C. for 30 mins. Stirring stopped and phases were allowed to separate, the lower DCM phase was drained off and transferred to a holding vessel. The DCM solution of C108 was then held to be analysed and taken forward into the next step without isolation of C108 assuming 100% yield for the next step (Step 3).

The relative amount of each reagent used in Step 2 of this example compares to the limiting reagent, which is C107.

### Step 3. Preparation of Methyl(S)-2-(chloromethyl)-1-(oxetan-2-ylmethyl)-1H-benzo[d]imidazole-6-carboxylate (C109)

To a Jacketed vessel with overhead stirring and nitrogen purged was added the DCM solution of C108 from previous step (Step 2) (Total volume=˜1350 mL, Theory=75.5 g, 319.5 mmol, limiting reagent) and stirred at 20° C. Under vacuum the reaction contents were concentrated to a total volume of 3 to 4 mL/g (226-301 mL). The vacuum was released and next was charged Isopropanol (8.0 mL/g, 474.7 g). The mixture was again concentrated to a total volume of 3 to 4 mL/g (226-301 mL). To the mixture at 25° C. was added 2-Chloro-1,1,1-trimethoxyethane (1.05 equiv., 51.87 g, 45.22 mL, 335.5 mmol) in one portion. To the reaction was added Citric Acid (0.01 equiv., 0.6139 g, 3.195 mmol) in one portion. The reaction was then warmed to 50° C. Upon reaction completion the temperature was reduced to 30° C. before the addition of water (12.0 mL/g, 906.0 g, 906.0 mL) over 1 hour. At end of the water addition the mixture was a fine suspension which was held at 30° C. for 30 mins before cooling to 5° C. After 16 hours at 5° C. the suspension was filtered and the solids washed with IPA/Water (1:9 vol ratio) (3.0 mL/g, 226.5 mL). The filter cake was pulled dry under vacuum for 2 hours and then placed in a vac oven to dry at 40° C./for at least 12 hours affording 61.6 g (65%-2 steps) of C109 as a white solid.

¹H NMR (600 MHZ, CDCl₃) δ 8.12 (s, 1H), 8.00 (d, 1H), 7.79 (d, 1H), 5.16-5.26 (m, 1H), 5.03 (s, 2H), 4.57-4.66 (m, 2H), 4.48-4.56 (m, 1H), 4.33 (m, 1H), 3.95 (s, 3H), 2.71-2.81 (m, 1H), 2.36-2.47 (m, 1H).

The relative amount of each reagent used in Step 3 of this example compares to the limiting reagent, which is C108.

### Example 7. Preparation of Methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate (C110)

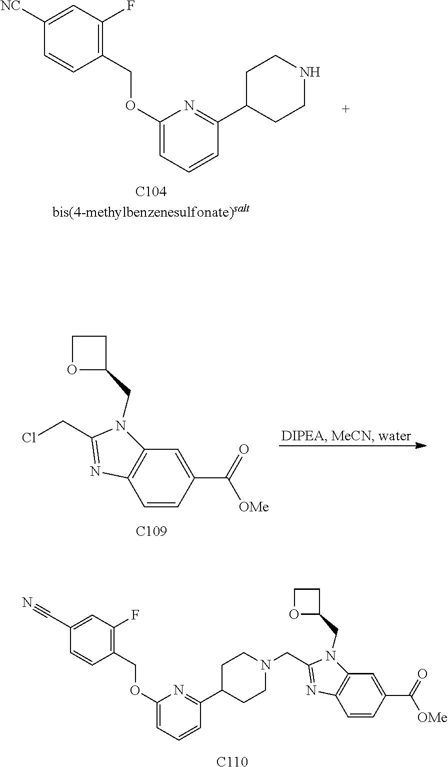 [SMILES: N#Cc1ccc(COc2cccc(C3CCNCC3)n2)c(F)c1 ; "C104bis(4-methylbenzenesulfonate)salt" ; COC(=O)c1ccc2nc(CCl)n(C[C@@H]3CCO3)c2c1 ; "C109" ; COC(=O)c1ccc2nc(CN3CCC(c4cccc(OCc5ccc(C#N)cc5F)n4)CC3)n(C[C@@H]3CCO3)c2c1 ; "C110"]

To a vessel was added Acetonitrile (400 mL, 8 L/Kg) and the solvent was held at 25° C. To the vessel was added C104 bis(4-methylbenzenesulfonate) salt (57.3 g, 0.087 mol, 0.515 eq) maintaining a temperature of 25° C.±5.0° C. To the vessel was slowly added Diisopropylethylamine (74.0 mL, 0.424, 2.5 eq.) over at least 30 minutes keeping temperature at 25° C.±5.0° C. To the vessel was added the second charge of C104 bis(4-methylbenzenesulfonate) salt (57.3 g, 0.087 mol, 0.515 eq) maintaining a temperature of 25° C.±5.0° C. To the vessel was slowly added the second charge of Diisopropylethylamine (74.0 mL, 0.424, 2.5 eq.) over at least 30 minutes while maintaining a temperature of 25° C.±5.0° C. To the vessel was added C109 (50.0 g, 0.170 mol, 1.00 eq., limiting reagent). The reaction mixture was heated to 50° C.±5.0° C. over 30 minutes. This mixture was held at 50° C.±5.0° C. for at least 6 h. A sample was removed for UPLC analysis. To the vessel was added C110 seed (0.5 g, 0.88 mmol, 0.01 g/g). Reaction mixture was left to granulate for 2 h at 50° C. To the vessel was added Water (600 mL, 12 L/Kg) over at least 1 h maintaining a temperature of 50° C. (Temperature of 40 to 55° C. acceptable). Cooled to 15° C.±5.0° C. over 3 h (rate of 0.2° C./min). The slurry was stirred at 15° C. for at least 8 h. The reaction mixture was filtered. To the empty vessel was added Water (175 mL, 3.5 mL/g). To the vessel was added Acetonitrile (25 mL, 0.5 mL/g). The wash solution was stirred at 15° C.±5.0° C. The wash solution of acetonitrile and water was transferred onto the filter. The product was pulled dry and loaded into a vacuum oven. The product was dried under vacuum for at least 12 h at 50° C. (93.7 g, 97% yield).

¹H NMR (600 MHZ, DMSO-d6) δ 8.28 (s, 1H), 7.87 (d, 1H), 7.80 (d, 1H), 7.55-7.73 (m, 4H), 6.87 (d, 1H), 6.70 (d, 1H), 5.45 (s, 2H), 5.04-5.19 (m, 1H), 4.81 (dd, 1H), 4.66 (dd, 1H), 4.41-4.54 (m, 1H), 4.36 (dt, 1H), 3.94 (d, 1H), 3.86 (s, 3H), 3.76 (d, 1H), 2.97 (d, 1H), 2.82 (d, 1H), 2.63-2.77 (m, 1H), 2.49-2.63 (m, 1H), 2.37-2.46 (m, 1H), 2.18-2.29 (m, 1H), 2.05-2.18 (m, 1H), 1.47-1.82 (m, 4H). LC-MS (ES+): 570.5 (M+H).

The relative amount of each reagent used in this example compares to the limiting reagent, which is C109.

A sample of C110 (as prepared by a procedure as described in Example 7) was used to generate the powder X-ray diffraction pattern and was found to be crystalline. An observed PXRD pattern for the crystalline C110 (herein designated as Form X) is provided in FIG. 14 and the corresponding peak list is provided in Table E7 (with peaks the relative intensity of which is 5% or greater).

TABLE E7
PXRD Peak Table for crystalline C110 (Form X)

| Angle, 2Theta (2θ) | Relavtive Intensity, % |
|---|---|
| 7.5 | 9.5 |
| 7.6 | 8.9 |
| 8.4 | 28.3 |
| 10.7 | 21.3 |
| 11.9 | 100.0 |
| 12.0 | 64.1 |
| 13.7 | 22.9 |
| 15.0 | 11.0 |
| 16.5 | 26.0 |
| 16.9 | 46.2 |
| 17.0 | 53.8 |
| 18.2 | 15.5 |
| 18.6 | 20.5 |
| 18.9 | 70.6 |
| 19.2 | 62.3 |
| 19.5 | 26.9 |
| 19.7 | 15.9 |
| 20.0 | 43.9 |
| 20.3 | 19.4 |
| 20.5 | 23.3 |
| 21.4 | 15.6 |
| 22.0 | 10.4 |
| 22.4 | 39.0 |
| 22.6 | 29.3 |
| 22.8 | 35.2 |
| 22.9 | 45.6 |
| 23.3 | 6.6 |
| 23.8 | 84.2 |
| 24.2 | 11.8 |
| 25.2 | 26.7 |
| 25.4 | 32.9 |
| 25.6 | 46.1 |
| 26.4 | 6.0 |
| 27.2 | 25.9 |
| 27.7 | 29.9 |
| 29.2 | 5.6 |
| 29.5 | 18.2 |

### Preparation of C110 Seed Materials

A seed material of C110 can be prepared by a similar procedure as described in this example except for not using a seed material (i.e., relying on self-nucleate).

### Example 7A. An Alternative Preparation of Methyl(S)-2-((4-(6-((4-cyano-2-fluorobenzyl)oxy)pyridin-2-yl) piperidin-1-yl)methyl)-1-(oxetan-2-ylmethyl)-1H-benzo[d]imidazole-6-carboxylate (C110)

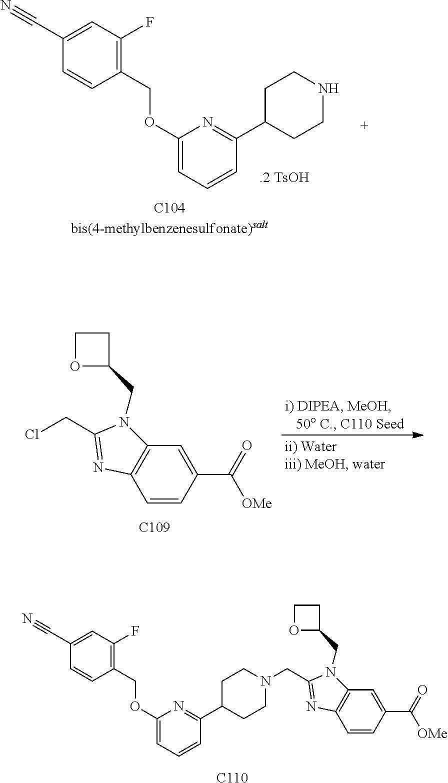 [SMILES: N#Cc1ccc(COc2cccc(C3CCNCC3)n2)c(F)c1 ; "C104bis(4-methylbenzenesulfonate)salt" ; COC(=O)c1ccc2nc(CCl)n(C[C@@H]3CCO3)c2c1 ; "C109" ; COC(=O)c1ccc2nc(CN3CCC(c4cccc(OCc5ccc(C#N)cc5F)n4)CC3)n(C[C@@H]3CCO3)c2c1 ; "C110"]

To the vessel was added Methanol (1200 mL, 12 L/kg) and the solvent was held at 25° C. To the vessel was added C104 bis(4-methylbenzenesulfonate) salt (244.8 g, 373 mol, 1.1 eq.) at 25° C. To the vessel was slowly added diisopropylethylamine (297 mL, 1697 mol, 5.0 eq.) over at least 15 minutes keeping temperature at 25° C. To the vessel was added C109 (100.0 g, 339 mol, 1.0 eq., limiting reagent) and the reaction was heated to 50° C. This mixture was held at 50° C. for 15 minutes. To the vessel was added C110 seed (1.0 g, 1.8 mmol, 0.01 kg/kg). This mixture was held at 50° C. for at least 24 h. A sample was removed for UPLC analysis. To the vessel was added Water (400 mL, 4 L/kg) over at least 1 h maintaining a temperature of 50° C. (Temperature of 45 to 55° C. acceptable). The reaction mixture was granulated at 50° C. for 1 h. Cooled to 20° C. at a rate of 0.2 K/min. The slurry was stirred at 20° C. for at least 2 h. The reaction was filtered. To the empty vessel was added Water (350 mL, 3.5 L/kg). To the vessel was added Methanol (50 mL, 0.5 L/kg). The wash solution was stirred at 20° C. The wash solution of methanol and water was transferred onto the filter. To the empty vessel was added Water (350 mL, 3.5 L/kg). To the vessel was added Methanol (50 mL, 0.5 L/kg). The wash solution was stirred at 20° C. The wash solution of methanol and water was transferred onto the filter. The product was pulled dry on the filter for 2 h. The product was transferred to a vacuum oven and dried under vacuum for at least 16 h at 50° C. (200.6 g, 100% yield). Comparing to the process in Example 7, the process 7A reduced impurity IMP-A in the final product C110.

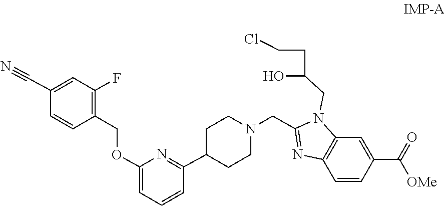 [SMILES: COC(=O)c1ccc2nc(CN3CCC(c4cccc(OCc5ccc(C#N)cc5F)n4)CC3)n(CC(O)CCCl)c2c1]

The relative amount of each reagent used in this example compares to the limiting reagent, which is C109.

### Preparation of C110 Seed Materials

A seed material of C110 can be prepared by a similar procedure as described in this example except for not using a seed material (i.e., relying on self-nucleate). Alternatively, a seed material of C110 can be prepared by a similar procedure as described in Example 6.

### Example 8. Preparation of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid hemi-barium salt

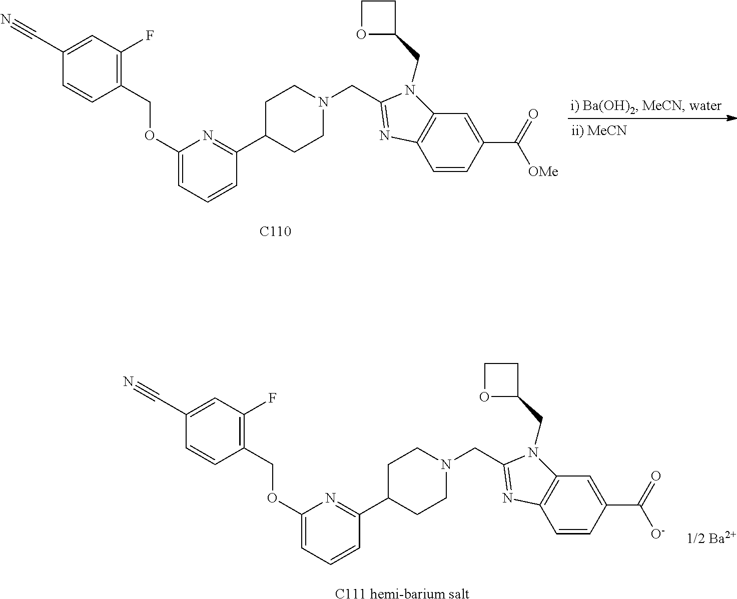

To a 100 ml reaction flask was charged C110 (17.56 mmol, 10.00 g, limiting reagent) followed by the addition of Acetonitrile (120 ml, 12 ml/g). To this was then added Barium Hydroxide (3.11 g, 0.55 eq.). To this was added Water (25 ml, 2.5 ml/g). The resulting reaction was then heated to a reaction temperature of 60° C. and was left to stir at 60° C. overnight. On reaching reaction completion the reaction cooled to 25° C. to await isolation. The reaction was filtered and washed with 3× Acetonitrile (3×20 ml, 3×2 ml/g) and the resulting solid C111 hemi-barium salt dried in a vacuum oven at 50° C. overnight (10.47 g, 95.7% yield). ICP-MS quantification of Ba % w/w afforded relative ratio of Ba to C111 in the salt.

The relative amount of each reagent used in this example compares to the limiting reagent, which is C110.

| | |
|---|---|
| Salt Stoichiometry | C111:Ba = 1.0:0.5 |
| ¹H NMR | ¹H NMR (400 MHz, Acetic acid-d4) δ = 8.35 (d, J = 0.9 Hz, 1H), 8.07 |
| | (dd, J = 1.5, 8.6 Hz, 1H), 7.87 (d, J = 8.6 Hz, 1H), 7.74-7.67 (m, 1H), |
| | 7.66 (d, J = 7.0 Hz, 1H), 7.59-7.54 (m, 1H), 7.56 (s, 1H), 6.94 (d, J = |
| | 7.2 Hz, 1H), 6.80 (d, J = 8.0 Hz, 1H), 5.49 (s, 2H), 5.37 (dq, J = 2.3, 7.3 |
| | Hz, 1H), 4.95 (d, J = 15.1 Hz, 1H), 4.89 (dd, J = 7.3, 16.0 Hz, 1H), 4.87 |
| | (d, J = 15.1 Hz, 1H), 4.81-4.72 (m, 2H), 4.54 (td, J = 6.0, 9.3 Hz, 1H), |
| | 3.90 (s, 1H), 3.77 (br s, 1H), 3.52-3.37 (m, 2H), 3.06 (tt, J = 3.4, 11.5 |
| | Hz, 1H), 2.92-2.81 (m, 1H), 2.56 (dddd, J = 7.0, 7.3, 9.3, 12.0 Hz, 1H), |
| | 2.30-2.14 (m, 2H), 2.12-1.96 (m, 3H) |
| ¹³C NMR | ¹³C {¹H} NMR (101 MHz, Acetic acid-d4) δ = 169.65, 162.31, 159.52, |
| | 159.76 (d, J = 248.6 Hz, 1C), 147.47, 144.43, 140.73, 135.00, 131.24 |
| | (d, J = 14.4 Hz, 1C), 130.72 (d, J = 4.5 Hz, 1C), 128.78 (d, J = 4.1 Hz, |
| | 1C), 126.38, 125.20, 119.31, 119.05, 117.77 (d, J = 3.3 Hz, 1C), |
| | 115.40, 113.71, 112.12 (d, J = 9.8 Hz, 1C), 108.89, 81.56, 69.81, 60.73 |
| | (d, J = 4.1 Hz, 1C), 53.59 (br s, 1C), 53.23, 51.36, 49.14, 39.57, 28.55, |
| | 28.35, 23.87 |
| MS | Measured m/z 556.2356, calculated m/z 556.2355 |

A sample of the hemi-barium salt of C111 (as prepared by a procedure as described in Example 8) was used to generate the powder X-ray diffraction pattern. The PXRD pattern for the hemi-barium salt of C111 (as prepared by a procedure as described in Example 8) is provided in FIG. 8.

### Example 9. Preparation of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid, Tris Salt (Tris Salt of C111)

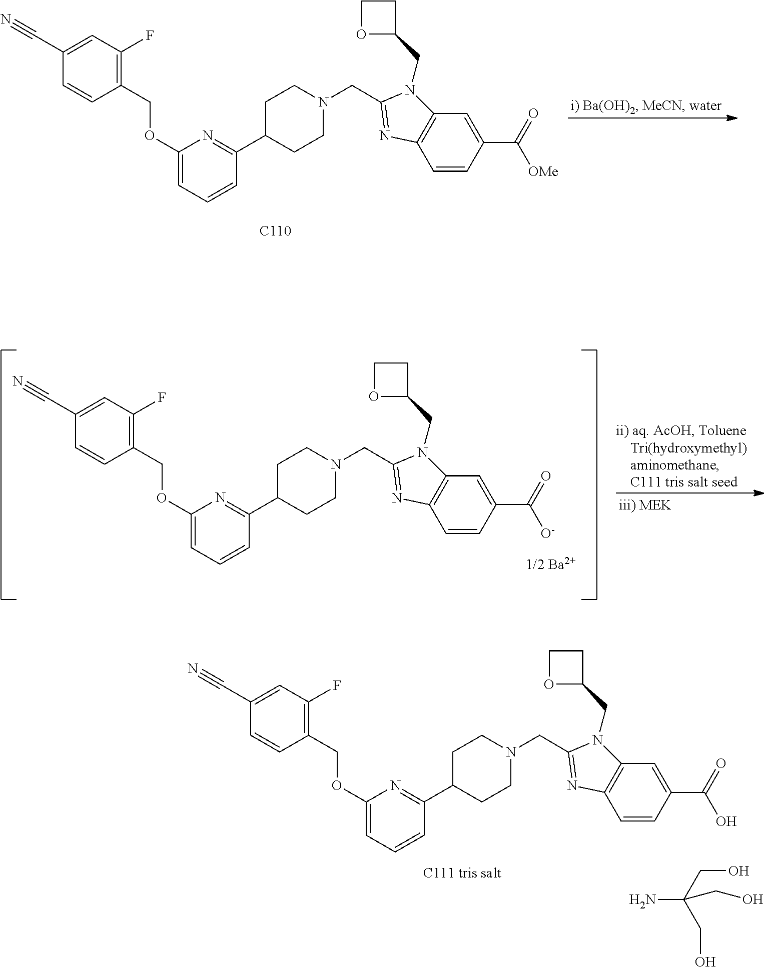

To a 2 L reactor ready was added H₂O (1.5 mL/g; 105 mL). To the vessel was then added MeCN (7 mL/g; 490 mL) in one portion. To the vessel was added Ba(OH)₂ (0.5 equivs; 10.53 g; 61.44 mmol) as a solid and the vessel was stirred to ensure suspension of the barium hydroxide. To the same vessel was added C110 (70.0 g; 122.9 mmol, limiting reagent) as a solid in one portion with small amount of MeCN used to rinse the addition funnel. The reaction then heated to a contents temperature of 55° C. at a rate of 1° C./min. The reaction was held under these conditions for a total of 24 hrs under N₂ (g). A sample of the reaction taken to check for reaction completion (LC). Upon reaction completion, the reaction mixture was then cooled to a contents temperature of 25° C. at a rate of 1° C./min. To this slurry is added H₂O (3.5 mL/g; 245 ml). To the slurry is added Toluene (1 ml/g; 70 ml). To this slurry is added Acetic Acid (1.5 equivs; 10.56 mL; 184.3 mmol). The resulting mixture is stirred for 30 minutes at 25° C. Phases separated. The organic solution was heated to 45° C. To the organic solution was added 2-Amino-2-(Hydroxymethyl)-1,3-propanediol (1.20 equivs; 18.05 g; 147.5 mmol) as a solution in water (1 mL/g; 70 mL). A seed loading of 0.004 g/g of C111 tris salt was added to the solution (0.280 g; 0.413 mmol; 0.003 molar equivs). The reaction slurry held at 45° C. for 60 minutes. The reaction cooled to 25° C. at a rate of 1° C./min and then held for 60 mins. The reaction heated to 45° C. at a rate of 1° C./mins and then held for 60 mins. The reaction cooled to 20° C. at a rate of 1° C./min and held overnight. The slurry was isolated by filtration and the liquors were pulled through to the top of the cake. Cake was washed with first MEK (3 mL/g; 210 ml, methyl ethyl ketone) wash and pulled through to the top of the cake. Cake was washed with second MEK (3 mL/g; 210 ml) wash and pulled through to the top of the cake. The resulting cake pulled dry for 30 minutes. The solids (C111 tris salt) were off-loaded and dried in a vacuum oven at 50° ° C. overnight (73.01 g; 107.9 mmol; ˜ 87% yield).

The final product (C111 tris salt) prepared in this example contains reduced amount of Impurity IMP-2 comparing to the method in Example 4A-01 of U.S. Pat. No. 10,208,019.

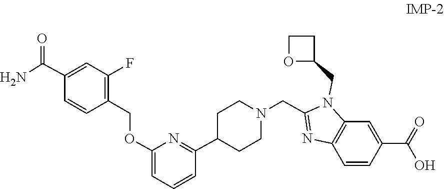 [SMILES: NC(=O)c1ccc(COc2cccc(C3CCN(Cc4nc5ccc(C(=O)O)cc5n4C[C@@H]4CCO4)CC3)n2)c(F)c1]

The relative amount of each reagent used in this example compares to the limiting reagent, which is C110.

### Preparation of Tris Salt of C111 Seed Materials

A seed material of Tris Salt of C111 can be prepared by a similar procedure as described in this example except for not using a seed material (i.e., relying on self-nucleate).

### Example 10. An Alternative Preparation of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid tris salt (Tris Salt of C111)

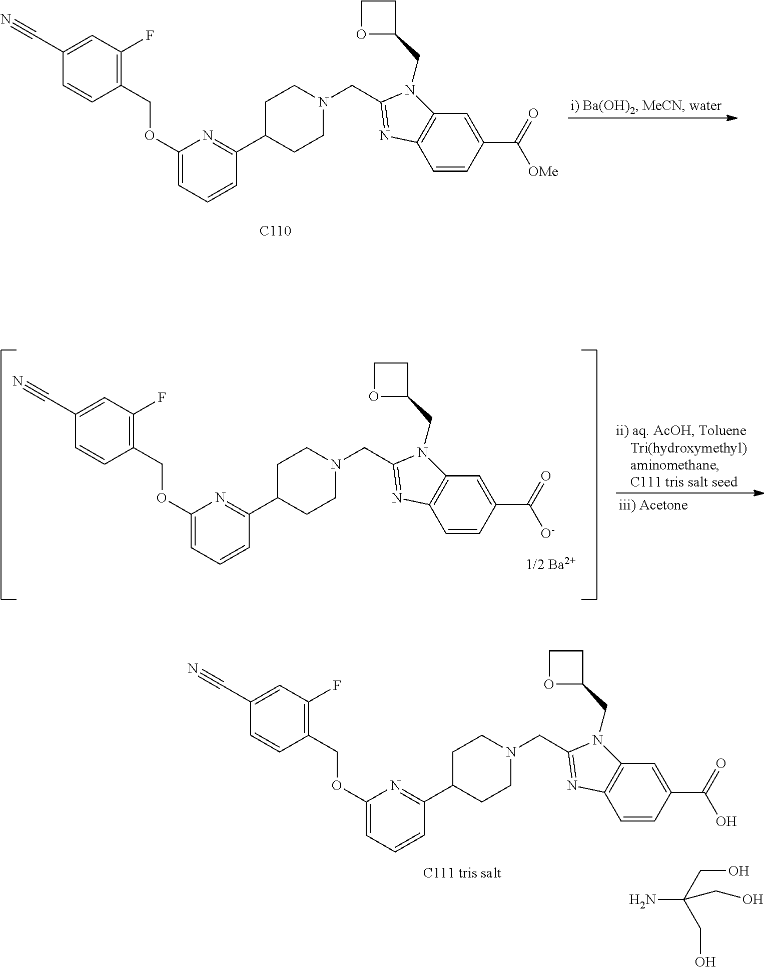

To the vessel was added Acetone (6.0 L/kg; 300 mL) in one portion. To the same vessel was added C110 (50.0 g; 87.7 mmol) as a solid in one portion. To the vessel was added Ba(OH)₂ (0.5 eq.; 7.52 g; 43.89 mmol) as a solid and the slurry was stirred to ensure suspension of the barium hydroxide. To the vessel was added H₂O (1.2 L/kg; 60 mL) in one portion. The reaction then heated to a contents temperature of 50° C. at a rate of 1° C./min. The reaction was held under these conditions for a total of 20-36 hrs under N₂ (g). A sample of the reaction mixture was taken to check for reaction completion (LC). Upon reaction completion, the reaction mixture was cooled to a contents temperature of 25° C. at a rate of 1° C./min. To this slurry is added H₂O (3.0 L/kg; 150 ml). To the slurry is added Toluene (3.5 L/kg; 175 ml). To this slurry is added Acetic Acid (1.5 eq.; 7.55 mL; 131.7 mmol). The resulting mixture is stirred for not less than 1 hour at 25° C. Phases separated. To the organic phase was added Methanol (0.75 L/kg; 37.5 ml). The organic solution was heated to 45° C. To the organic solution was added 2-Amino-2-(Hydroxymethyl)-1,3-propanediol (1.20 equivs; 12.76 g; 105.3 mmol) as a solution in water (1 L/kg; 50 mL). A seed loading of 0.004 g/g of C111 tris salt was added to the solution (0.20 g; 0.296 mmol; 0.003 equivs). The reaction slurry held at 45° C. for 60 minutes. The reaction cooled to 20° C. at a rate of 0.2° C./min and then held overnight. The slurry was isolated by filtration and the liquors were pulled through to the top of the cake. Cake was washed with first Acetone (3 L/kg; 150 ml) wash and pulled through to the top of the cake. Cake was washed with second Acetone (3 L/kg; 150 ml) wash and pulled through to the top of the cake. The resulting cake pulled dry for 30 minutes. The solids were off-loaded and dried in a vacuum oven at 50° C. overnight (52.0 g, 88% yield).

The final product (C111 tris salt) prepared in this example contains reduced amount of Impurity IMP-2 comparing to the method in Example 4A-01 of U.S. Pat. No. 10,208,019.

The relative amount of each reagent used in this example compares to the limiting reagent, which is C110.

### Preparation of Tris Salt of C111 Seed Materials

A seed material of Tris Salt of C111 can be prepared by a similar procedure as described in this example except for not using a seed material (i.e., relying on self-nucleate). Alternatively, a seed material of Tris Salt of C111 can be prepared by a similar procedure as described in Example 9.

### Example 11. Preparation of Form 1 of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid, Tris Salt (Form 1 of Tris Salt of C111)

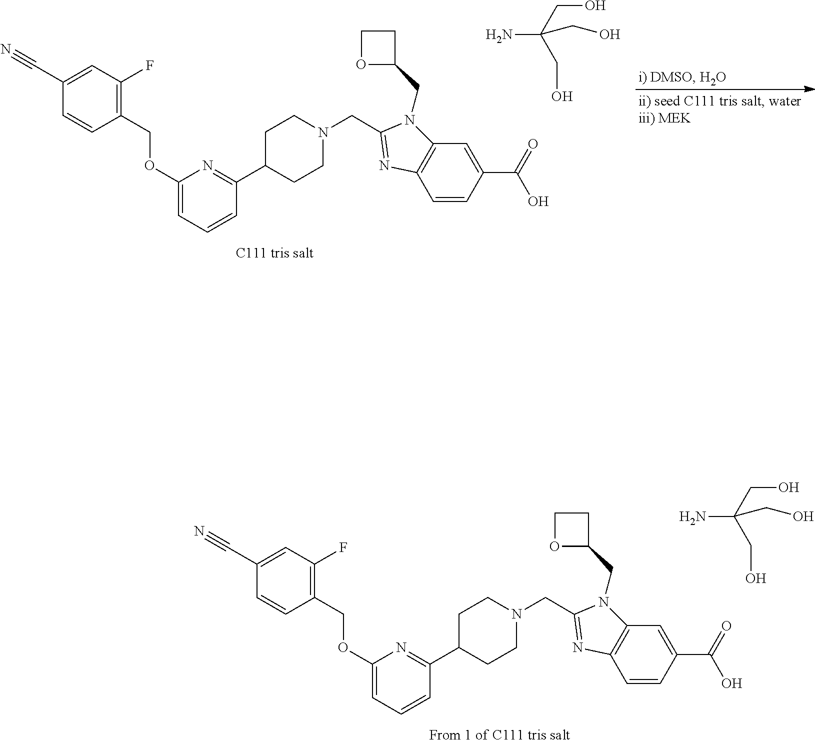 [SMILES: N#Cc1ccc(COc2cccc(C3CCN(Cc4nc5ccc(C(=O)O)cc5n4C[C@@H]4CCO4)CC3)n2)c(F)c1 ; NC(CO)(CO)CO ; N#Cc1ccc(COc2cccc(C3CCN(Cc4nc5ccc(C(=O)O)cc5n4C[C@@H]4CCO4)CC3)n2)c(F)c1 ; NC(CO)(CO)CO]

To a vessel was added DMSO (16 mL, 8 L/Kg) and the solvent was held at 25° C. To the vessel was added Water (2 mL, 1 L/Kg) and the solvent was held at 25° C. To the vessel was added C111 tris salt (2.0 g, 2.96 mmol, 1.0 eq, limiting reagent) maintaining a temperature of 25° C.±5.0° C. The reaction was heated to 65° C. over 30 minutes. The reaction was held at 65° C. for a minimum of 30 minutes. To the vessel was slowly added Water (2 mL, 1 L/Kg) over 30 minutes maintaining the temperature at 65° C. The reaction was held at 65° C. for a minimum of 30 minutes. To the vessel was added a seed C111 tris salt (10 mg, 0.01 mmol, 0.005 g/g) maintaining a temperature of 65° C. The reaction was held at 65° C. for a minimum of 1 hour. The reaction was cooled to 30° C. at a rate of 0.2° C./min. The reaction was held at 30° C. for 1 hour. The reaction was heated to 45° C. over 30 minutes. The reaction was held at 45° C. The reaction was cooled to 15° C. at a rate of 0.2° C./min. The reaction was left to granulate at 15° C. for at least 8 hours. The batch was filtered and pulled dry. To the vessel was added Butan-2-one (or methyl ethyl ketone, MEK) (6 mL, 3 L/Kg) and the solvent was cooled to 15° C. The cake wash was transferred onto the filter and the batch pulled dry. The product was transferred to the oven and dried at 55° C. for at least 8 hours.

The final product (C111 tris salt) prepared in this example contains reduced amount of Impurities IMP-1 and IMP-2 comparing to the method in Example 4A-01 of U.S. Pat. No. 10,208,019.

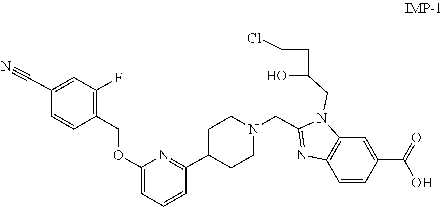 [SMILES: N#Cc1ccc(COc2cccc(C3CCN(Cc4nc5ccc(C(=O)O)cc5n4CC(O)CCCl)CC3)n2)c(F)c1]

### Preparation of Form 1 of Tris Salt of C111 Seed Materials

A seed material of Form 1 of Tris Salt of C111 can be prepared by a similar procedure as described in this example except for not using a seed material (i.e., relying on self-nucleate).

### Example 12. An alternative Preparation of Form 1 of(S)-2-((4-(6-((4-Cyano-2-fluorobenzyl)oxy)pyridin-2-yl) piperidin-1-yl)methyl)-1-(oxetan-2-ylmethyl)-1H-benzo[d]imidazole-6-carboxylic acid, Tris Salt (Form 1 of Tris Salt of C111)

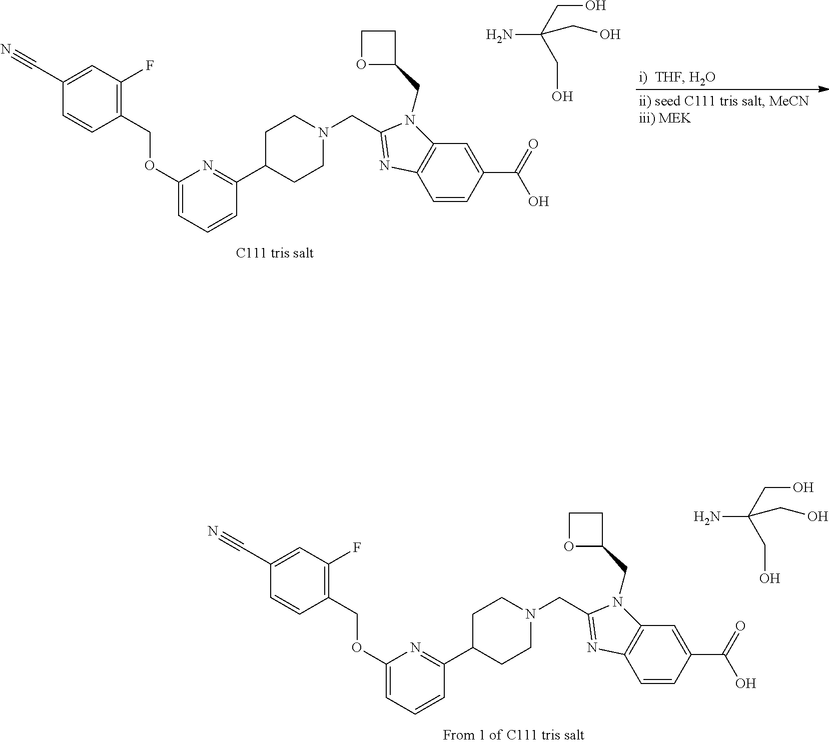 [SMILES: N#Cc1ccc(COc2cccc(C3CCN(Cc4nc5ccc(C(=O)O)cc5n4C[C@@H]4CCO4)CC3)n2)c(F)c1 ; NC(CO)(CO)CO ; N#Cc1ccc(COc2cccc(C3CCN(Cc4nc5ccc(C(=O)O)cc5n4C[C@@H]4CCO4)CC3)n2)c(F)c1 ; NC(CO)(CO)CO]

4.5 g C111 tris salt (limiting reagent), 18.75 ml THF (4.17 L/kgW) and 6.25 ml water (1.39 L/kgW) were charged to a vessel and agitated. The slurry was heated to 55° C. and checked for dissolution. The mixture was then cooled to 49° C. at 1K/min and contents was checked to remain a solution. To the solution was added 1% of a seed of C111 tris salt. The mixture was held at 49° C. for 1 hour after seeding and checked for slurry. The mixture was cooled to 35° C. at 0.1-0.2 K/min, and then was held at 35° C. for 1 hour. 25 ml of Acetonitrile (5.56 L/kgW) was added over 1.5 hours. The resulting mixture was held at 35° C. for 1 hour, and then cooled to 10° C. at 0.2 K/min. The mixture was held at 10° C. for at least 8 hours. Take a sample of the slurry to check particle size. If the D90 of the particles in the slurry is 125 microns or greater, then perform high shear wet milling. Repeated the sampling for particle size check and wet milling steps a few times until the D90 of particles in the slurry is below 125 microns. Then the slurry was filtered under pressure and wash with 2×10 ml MEK (2×2.22 L/kg) with agitation. The material was transferred to dryer and dried at 55° C. jacket temperature under reduced pressure (4.14 g, 92%).

The final product (C111 tris salt) prepared by the method in this example contains reduced amount of Impurities IMP-1, IMP-2, and IMP-3 comparing to the method in Example 4A-01 of U.S. Pat. No. 10,208,019. Moreover, the process in this example provided the final products in larger particle size and the particle size was relatively uniform (comparing to, for example, the method in in Example 4A-01 of U.S. Pat. No. 10,208,019).

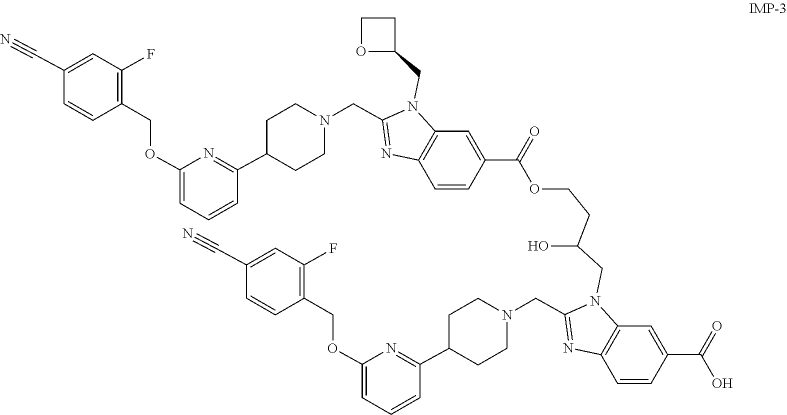 [SMILES: N#Cc1ccc(COc2cccc(C3CCN(Cc4nc5ccc(C(=O)O)cc5n4CC(O)CCOC(=O)c4ccc5nc(CN6CCC(c7cccc(OCc8ccc(C#N)cc8F)n7)CC6)n(C[C@@H]6CCO6)c5c4)CC3)n2)c(F)c1]

### Preparation of Form 1 of Tris Salt of C111 Seed Materials

A seed material of Form 1 of Tris Salt of C111 can be prepared by a similar procedure as described in this example except for not using a seed material (i.e., relying on self-nucleate). Alternatively, a seed material of Form 1 of Tris Salt of C111 can be prepared by a similar procedure as described in Example 11.

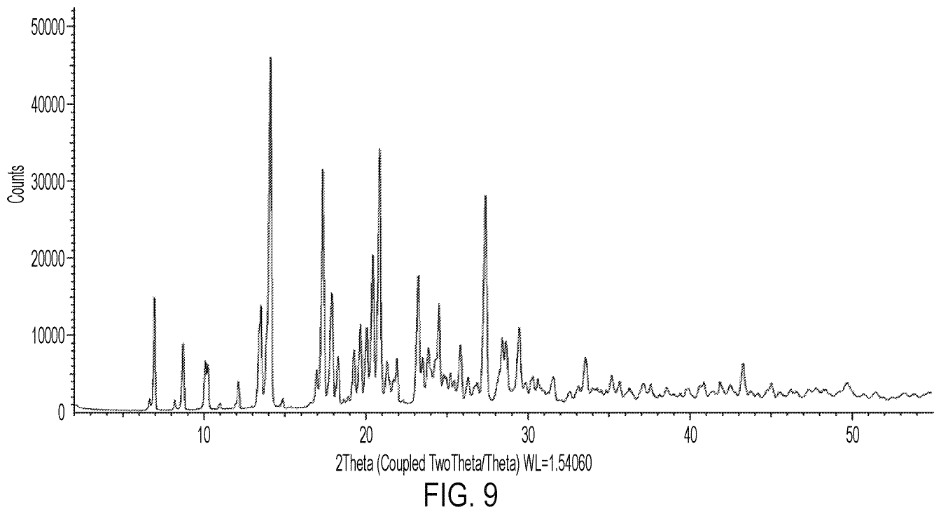

A sample of Form 1 of Tris Salt of C111 (as prepared by a procedure as described in Example 11 or 12) was used to generate the powder X-ray diffraction pattern. The PXRD pattern for the Form 1 of Tris Salt of C111 (as prepared by a procedure as described in Example 11 or 12) is provided in FIG. 9 and the corresponding peak list is provided in Table E12 (with peaks the relative intensity of which is 5% or greater).

TABLE E12
PXRD peak list for Form 1 of Tris Salt of C111

| Angle (°2θ) | Rel. Intensity (%) |
|---|---|
| 7.1 | 31 |
| 8.9 | 18 |
| 10.3 | 13 |
| 10.4 | 12 |
| 12.3 | 8 |
| 13.7 | 28 |
| 14.3 | 100 |
| 17.1 | 10 |
| 17.5 | 65 |
| 18.0 | 31 |
| 18.4 | 14 |
| 19.4 | 15 |
| 19.8 | 22 |
| 20.2 | 22 |
| 20.6 | 41 |
| 21.0 | 70 |
| 21.5 | 11 |
| 21.9 | 7 |
| 22.1 | 12 |
| 23.4 | 35 |
| 23.6 | 12 |
| 24.0 | 15 |
| 24.4 | 12 |
| 24.7 | 27 |
| 25.0 | 7 |
| 25.1 | 7 |
| 25.4 | 8 |
| 25.6 | 5 |
| 26.0 | 15 |
| 26.5 | 6 |
| 27.0 | 5 |
| 27.5 | 56 |
| 28.6 | 19 |
| 28.8 | 16 |
| 29.6 | 20 |
| 30.0 | 5 |

### Example 13. Solid State NMR Analysis of Form 1 of Tris Salt of C111

Solid state NMR (ssNMR) analysis [using a sample of Form 1 of Tris Salt of C111 as prepared by a procedure as described in Example 11 or 12] was conducted on a Bruker Avance III HD 400 MHz (¹H frequency) NMR spectrometer. A 4 mm magic angle spinning (MAS) probe at MAS rates of 10 KHz was used for the ¹³C analysis. A ¹⁹F spectrum was recorded using a 3.2 mm MAS probe with a spin rate of 20 KHz. ¹⁵N ssNMR analysis was conducted on a Bruker AVANCE NEO 400 MHZ NMR spectrometer equipped with a 4 mM MAS probe with a spin rate of 20 kHz. All spectra were acquired with the temperature was regulated to 20° C.

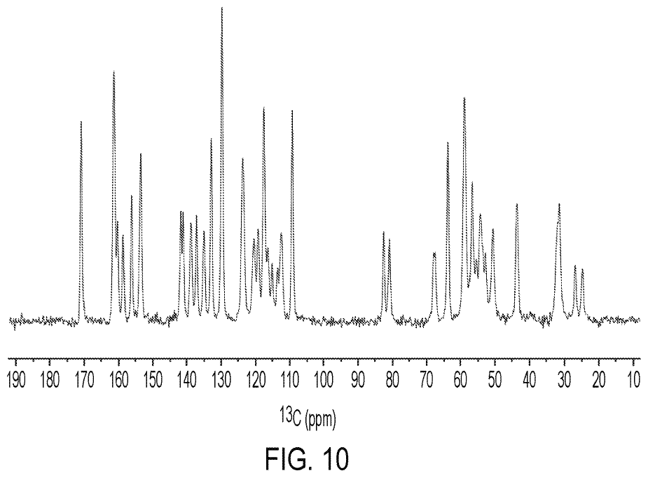

A ¹³C cross-polarization (CP) measurement with TOSS spinning sideband suppression was recorded with a 4 ms CP contact time and recycle delay of 40 s (see FIG. 10 and Table E13-a). A phase modulated proton decoupling field of ˜100 kHz was applied during spectral acquisition. Carbon spectral referencing is relative to neat tetramethylsilane, carried out by setting the high-frequency signal from an external sample of adamantane to 38.5 ppm.

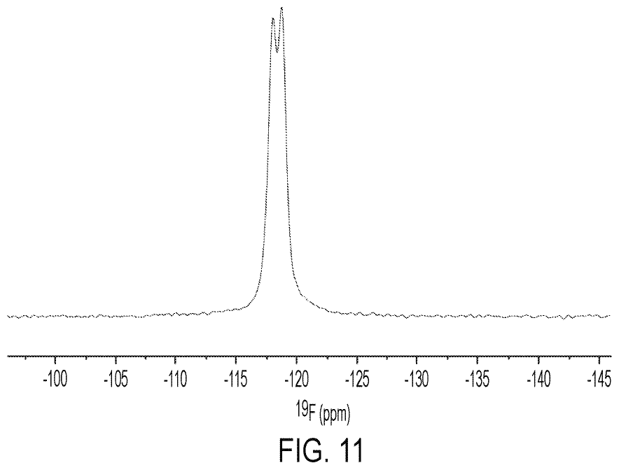

A ¹⁹F spectrum was collected by direct excitation with proton decoupling and a 120 s recycle delay (see FIG. 11 and Table E13-b). Spectral referencing is with respect to CFCl₃, carried out by setting the resonance from an external sample of 50% v/v trifluoroacetic acid in H₂O to −76.54 ppm.

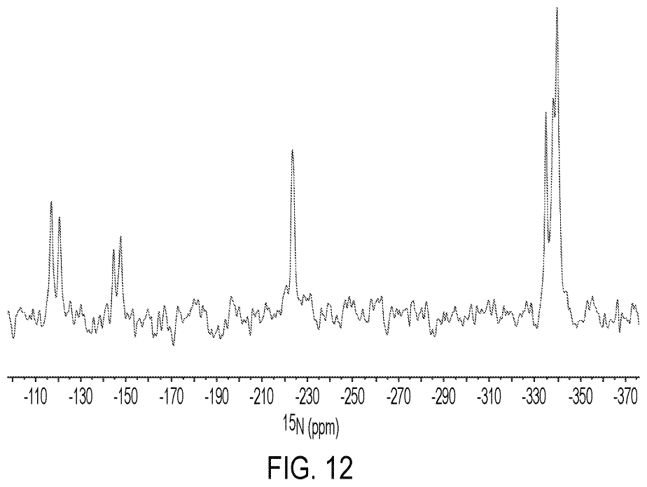

A ¹⁵N CP spectrum was recorded with a 10 ms CP contact time and a recycle delay of 3 s (see FIG. 12 and Table E13-c). Nitrogen spectral referencing is relative to neat nitromethane, carried out by setting the signal from an external sample of glycine to −346.8 ppm.

TABLE E13-a
¹³C ssNMR peak list for Form 1 of Tris Salt of C111.

| ¹³C Chemical Shifts δ (ppm) | Relative Intensity (%) |
|---|---|
| 24.7 | 16.6 |
| 26.9 | 17.6 |
| 31.5 | 37.4 |
| 43.9 | 37.3 |
| 50.9 | 29.3 |
| 53.0 | 21.7 |
| 54.6 | 34.2 |
| 55.8 | 19.5 |
| 57.0 | 44.4 |
| 59.2 | 71.2 |
| 64.0 | 57.0 |
| 67.8 | 21.9 |
| 68.2 | 21.6 |
| 81.1 | 26.1 |
| 82.7 | 28.4 |
| 109.4 | 67.2 |
| 112.6 | 28.0 |
| 113.7 | 16.7 |
| 115.4 | 18.2 |
| 116.6 | 23.1 |
| 117.7 | 68.1 |
| 119.4 | 29.3 |
| 120.7 | 26.0 |
| 123.9 | 51.9 |
| 130.0 | 100.0 |
| 133.1 | 58.4 |
| 135.2 | 28.6 |
| 137.4 | 33.7 |
| 139.0 | 31.3 |
| 141.3 | 34.8 |
| 141.9 | 34.9 |
| 153.6 | 53.4 |
| 156.3 | 39.9 |
| 158.8 | 27.4 |
| 160.4 | 31.8 |
| 161.5 | 79.7 |
| 171.0 | 63.7 |

TABLE E13-b
¹⁵N ssNMR peak list for Form 1 of Tris Salt of C111.

| ¹⁵N Chemical Shifts δ (ppm) | Relative Intensity (%) |
|---|---|
| −339.9 | 100.0 |
| −338.2 | 70.3 |
| −335.1 | 65.9 |
| −223.4 | 53.4 |
| −147.7 | 25.1 |
| −144.7 | 21.0 |
| −120.9 | 31.4 |
| −117.2 | 36.5 |

TABLE E13-c
¹⁹F ssNMR peak list for Form 1 of Tris Salt of C111.

| ¹⁹F Chemical Shifts δ (ppm) | Relative Intensity (%) |
|---|---|
| −118.8 | 100.0 |
| −118.1 | 96.6 |

Peak positions and relative intensities were obtained using ACD Labs Spectrus Processor 2019 software. The error in the reported peak positions in the ¹³C, ¹⁵N and ¹⁹F ssNMR data is estimated to be ±0.2 ppm. The ssNMR intensities can vary depending on the setup of the experimental parameters and the thermal history of the sample.

### Example 14. Raman Spectroscopy Analysis of Form 1 of Tris Salt of C111

Raman spectra [using a sample of Form 1 of Tris Salt of C111 as prepared by a procedure as described in Example 11 or 12] were collected using a RAM II FT-Raman module attached to a Vertex 70 spectrometer (Bruker Optik GmbH). The instrument was equipped with a 1064 nm solid-state (Nd:YAG) laser and a liquid nitrogen cooled germanium detector. Prior to data acquisition, instrument performance and calibration verifications were conducted using a white light source, and polystyrene and naphthalene references.

Samples were prepared and analyzed in truncated NMR tubes. A sample rotator (Ventacon, UK) was used during measurement to maximise the volume of material exposed to the laser during data collection. The backscattered Raman signal from the sample was optimised and data were collected at a spectral resolution of 2 cm-1 using a laser power of 500 mW. A Blackmann-Harris 4-term apodization function was applied to minimise spectral aberrations. Spectra were generated between 3500 and 50 cm-1 with the number of scans adjusted accordingly to ensure adequate signal to noise.

Spectra were normalized by setting the intensity of the most intense peak to 2.00. Peaks were then identified using the automatic peak picking function in the OPUS v8.2 software (Bruker Optik GmbH) with the sensitivity set to 2%. Peak positions and relative peak intensities were extracted and tabulated. The variability in the peak positions with this experimental configuration is within ±2 cm⁻¹.

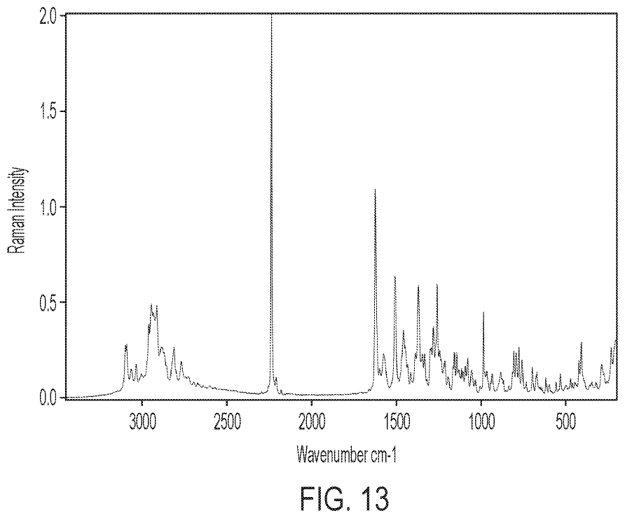

FIG. 13 shows a representative FT-Raman spectrum of Form 1 of Tris Salt of C111 collected and Table E14 shows the FT-Raman peak list for Form 1 of Tris Salt of C111

TABLE E14
FT-Raman peak list for Form 1 of Tris Salt of C111.

| Wavenumber (cm⁻¹) ± 2 cm⁻¹ | Relative Intensity (%) |
|---|---|
| 212 | 15.0 |
| 240 | 13.1 |
| 296 | 8.8 |
| 416 | 14.5 |
| 430 | 9.6 |
| 479 | 4.9 |
| 539 | 6.2 |
| 564 | 4.0 |
| 624 | 5.1 |
| 676 | 6.6 |
| 702 | 7.8 |
| 763 | 9.8 |
| 782 | 13.3 |
| 798 | 11.8 |
| 813 | 12.1 |
| 888 | 6.5 |
| 939 | 6.3 |
| 990 | 22.7 |
| 1060 | 7.1 |
| 1082 | 10.3 |
| 1096 | 8.1 |
| 1119 | 7.6 |
| 1146 | 11.7 |
| 1160 | 11.7 |
| 1197 | 5.5 |
| 1217 | 9.5 |
| 1261 | 29.6 |
| 1284 | 18.4 |
| 1334 | 11.6 |
| 1347 | 11.5 |
| 1371 | 29.1 |
| 1417 | 6.5 |
| 1459 | 17.5 |
| 1509 | 31.5 |
| 1576 | 11.4 |
| 1624 | 54.1 |
| 2205 | 5.2 |
| 2232 | 100.0 |
| 2761 | 9.3 |
| 2803 | 13.0 |
| 2878 | 13.1 |
| 2905 | 24.0 |
| 2937 | 24.3 |
| 2952 | 19.1 |
| 3026 | 8.7 |
| 3055 | 7.5 |
| 3082 | 13.8 |

Form 1 of Tris Salt of C111 can be identified by its unique solid state signatures with respect to, for example, powder X-ray diffraction (PXRD) data, solid state Nuclear Magnetic Resonance (ssNMR) data (e.g. ¹³C ssNMR data, ¹⁵N ssNMR data, and/or ¹⁹F ssNMR), and/or FT-Raman Spectroscopy data provided herein.

Form 1 of Tris Salt of C111 has a powder X-ray diffraction pattern (PXRD) comprising one peak or two peaks, in terms of 2θ (Cu Kα radiation source, wavelength of 1.5406 Å), selected from those at 14.3±0.2°, 17.5±0.2°, and 18.0±0.2°. In some embodiments, Form 1 of Tris Salt of C111 has a PXRD comprising one peak, in terms of 2θ, at 14.3±0.2°.

In some embodiments, Form 1 of Tris Salt of C111 has a PXRD comprising peaks, in terms of 2θ, at 14.3±0.2°, 17.5±0.2°, 18.0±0.2°, and 23.4±0.2°.

In some embodiments, Form 1 of Tris Salt of C111 has a PXRD peaks, in terms of 2θ, at 14.3±0.2°, 17.5±0.2°, 18.0±0.2°, 23.4±0.2°, and 24.7 0.2°. In a further embodiment, Form 1 of Tris Salt of C111 has a powder X-ray diffraction pattern (PXRD) substantially the same as FIG. 9 (FIG. 9).

In some embodiments, Form 1 of Tris Salt of C111 has a ¹³C ssNMR spectrum comprising one peak, in terms of chemical shifts, selected from those at 171.0±0.2 ppm and 141.3±0.2 ppm. In some further embodiments, Form 1 of Tris Salt of C111 has a ¹³C ssNMR spectrum. In some embodiments, Form 1 of Tris Salt of C111 has a ¹³C ssNMR spectrum comprising peaks, in terms of chemical shifts, at 171.0±0.2 ppm and 141.3±0.2 ppm.

In some embodiments, Form 1 of Tris Salt of C111 has a ¹³C ssNMR spectrum comprising peaks, in terms of chemical shifts, at 171.0±0.2 ppm, 141.3±0.2 ppm, and 64.0±0.2 ppm.

In some embodiments, Form 1 of Tris Salt of C111 has a ¹³C ssNMR spectrum comprising peaks, in terms of chemical shifts, at 171.0±0.2 ppm, 141.9±0.2 ppm, 141.3±0.2 ppm, 120.7±0.2 ppm, and 64.0±0.2 ppm. In a further embodiment, Form 1 of Tris Salt of C111 has a ¹³C ssNMR spectrum substantially the same as FIG. 10 (FIG. 10).

In some embodiments, Form 1 of Tris Salt of C111 has a ¹⁹F ssNMR spectrum comprising one peak, in terms of chemical shifts, at −118.8±0.2 ppm. In a further embodiment, the crystalline form has a ¹⁹F ssNMR spectrum substantially the same as FIG. 11 (FIG. 11).

In some embodiments, Form 1 of Tris Salt of C111 has a ¹⁵N ssNMR spectrum comprising one peak, in terms of chemical shifts, at −339.9±0.2 ppm or −223.4±0.2 ppm.

In some embodiments, Form 1 of Tris Salt of C111 has a ¹⁵N ssNMR spectrum comprising one peak, in terms of chemical shifts, at −339.9±0.2 ppm.

In some embodiments, Form 1 of Tris Salt of C111 has a ¹⁵N ssNMR spectrum comprising one peak, in terms of chemical shifts, at −223.4±0.2 ppm.

In some embodiments, Form 1 of Tris Salt of C111 has a ¹⁵N ssNMR spectrum comprising peaks, in terms of chemical shifts, at −339.9±0.2 ppm and −223.4±0.2 ppm. In a further embodiment, Form 1 of Tris Salt of C111 has a ¹⁵N ssNMR spectrum substantially the same as FIG. 12 (FIG. 12).

In some embodiments, Form 1 of Tris Salt of C111 has an FT-Raman spectrum comprising one peak or two peaks, in terms of wavenumbers (cm 1), selected from those at 1371±2 cm⁻¹, 430±2 cm⁻¹, and 416±2 cm⁻¹.

In some embodiments, Form 1 of Tris Salt of C111 has an FT-Raman spectrum comprising peaks, in terms of wavenumbers (cm⁻¹), at 1371±2 cm⁻¹, 430±2 cm⁻¹, and 416±2 cm⁻¹.

In some embodiments, Form 1 of Tris Salt of C111 has a FT-Raman spectrum comprising peaks, in terms of wavenumbers (cm⁻¹), at 1371±2 cm⁻¹, 430±2 cm⁻¹, 416±2 cm⁻¹, and 3026±2 cm⁻¹. In a further embodiment, In some embodiments, Form 1 of Tris Salt of C111 has an FT-Raman spectrum substantially the same as FIG. 13 (FIG. 13).

### Example AA. CHO GLP-1R Clone H6—Assay 1

GLP-1R-mediated agonist activity was determined with a cell-based functional assay utilizing an HTRF (Homogeneous Time-Resolved Fluorescence) CAMP detection kit (cAMP HI Range Assay Kit; CisBio cat #62AM6PEJ) that measures CAMP levels in the cell. The method is a competitive immunoassay between native cAMP produced by the cells and exogenous CAMP labeled with the dye d2. The tracer binding is visualized by a mAb anti-cAMP labeled with Cryptate. The specific signal (i.e. energy transfer) is inversely proportional to the concentration of CAMP in either standard or experimental sample.

The human GLP-1R coding sequence (NCBI Reference Sequence NP_002053.3, including naturally-occurring variant Gly168Ser) was subcloned into pcDNA3 (Invitrogen) and a cell line stably expressing the receptor was isolated (designated Clone H6). Saturation binding analyses (filtration assay procedure) using ¹²⁵I-GLP-17.36 (Perkin Elmer) showed that plasma membranes derived from this cell line express a high GLP-1R density (K_{d}: 0.4 nM, Bₘₐₓ: 1900 fmol/mg protein).

Cells were removed from cryopreservation, re-suspended in 40 mL of Dulbecco's Phosphate Buffered Saline (DPBS—Lonza Cat #17-512Q) and centrifuged at 800×g for 5 minutes at 22° C. The cell pellet was then re-suspended in 10 mL of growth medium [DMEM/F12 1:1 Mixture with HEPES, L-Gln, 500 mL (DMEM/F12 Lonza Cat #12-719F), 10% heat inactivated fetal bovine serum (Gibco Cat #16140-071), 5 mL of 100× Pen-Strep (Gibco Cat #15140-122), 5 mL of 100× L-Glutamine (Gibco Cat #25030-081) and 500 μg/mL Geneticin (G418) (Invitrogen #10131035)]. A 1 mL sample of the cell suspension in growth media was counted on a Becton Dickinson ViCell to determine cell viability and cell count per mL. The remaining cell suspension was then adjusted with growth media to deliver 2000 viable cells per well using a Matrix Combi Multidrop reagent dispenser, and the cells were dispensed into a white 384 well tissue culture treated assay plate (Corning 3570). The assay plate was then incubated for 48 hours at 37° C. in a humidified environment in 5% carbon dioxide.

Varying concentrations of each compound to be tested (in DMSO) were diluted in assay buffer (HBSS with Calcium/Magnesium (Lonza/BioWhittaker cat #10-527F)/0.1% BSA (Sigma Aldrich cat #A7409-1L)/20 mM HEPES (Lonza/BioWhittaker cat #17-737E) containing 100 μM 3-isobutyl-1-methylxanthin (IBMX; Sigma cat #15879). The final DMSO concentration is 1%.

After 48 hours, the growth media was removed from the assay plate wells, and the cells were treated with 20 μL of the serially diluted compound in assay buffer for 30 minutes at 37° C. in a humidified environment in 5% carbon dioxide. Following the 30 minute incubation, 10 μL of labeled d2 cAMP and 10 μL of anti-cAMP antibody (both diluted 1:20 in cell lysis buffer; as described in the manufacturer's assay protocol) were added to each well of the assay plate. The plates were then incubated at room temperature and after 60 minutes, changes in the HTRF signal were read with an Envision 2104 multi-label plate reader using excitation of 330 nm and emissions of 615 and 665 nm. Raw data were converted to nM cAMP by interpolation from a CAMP standard curve (as described in the manufacturer's assay protocol) and the percent effect was determined relative to a saturating concentration of the full agonist GLP-1₇₋₃₆ (1 μM) included on each plate. EC₅₀ determinations were made from agonist dose-response curves analyzed with a curve fitting program using a 4-parameter logistic dose response equation.

### Example BB. CHO GLP-1R Clone C6—Assay 2

GLP-1R-mediated agonist activity was determined with a cell-based functional assay utilizing an HTRF (Homogeneous Time-Resolved Fluorescence) CAMP detection kit (cAMP HI Range Assay Kit; Cis Bio cat #62AM6PEJ) that measures cAMP levels in the cell. The method is a competitive immunoassay between native cAMP produced by the cells and exogenous cAMP labeled with the dye d2. The tracer binding is visualized by a mAb anti-CAMP labeled with Cryptate. The specific signal (i.e. energy transfer) is inversely proportional to the concentration of CAMP in either a standard or an experimental sample.

The human GLP-1R coding sequence (NCBI Reference Sequence NP_002053.3, including naturally-occurring variant Leu260Phe) was subcloned into pcDNA5-FRT-TO and a clonal CHO cell line stably expressing a low receptor density was isolated using the Flp-In™ T-Rex™ System, as described by the manufacturer (ThermoFisher). Saturation binding analyses (filtration assay procedure) using ¹²⁵I-GLP-1 (Perkin Elmer) showed that plasma membranes derived from this cell line (designated clone C6) express a low GLP-1R density (K_{d}: 0.3 nM, Bₘₐₓ: 240 fmol/mg protein), relative to the clone H6 cell line.

Cells were removed from cryopreservation, re-suspended in 40 mL of Dulbecco's Phosphate Buffered Saline (DPBS—Lonza Cat #17-512Q) and centrifuged at 800×g for 5 minutes at 22° C. The DPBS was aspirated, and the cell pellet was re-suspended in 10 mL of complete growth medium (DMEM:F12 1:1 Mixture with HEPES, L-Gln, 500 mL (DMEM/F12 Lonza Cat #12-719F), 10% heat inactivated fetal bovine serum (Gibco Cat #16140-071), 5 mL of 100× Pen-Strep (Gibco Cat #15140-122), 5 mL of 100× L-Glutamine (Gibco Cat #25030-081), 700 μg/mL Hygromycin (Invitrogen Cat #10687010) and 15 μg/mL Blasticidin (Gibco Cat #R21001). A 1 mL sample of the cell suspension in growth media was counted on a Becton Dickinson ViCell to determine cell viability and cell count per mL. The remaining cell suspension was then adjusted with growth media to deliver 1600 viable cells per well using a Matrix Combi Multidrop reagent dispenser, and the cells were dispensed into a white 384 well tissue culture treated assay plate (Corning 3570). The assay plate was then incubated for 48 hours at 37° C. in a humidified environment (95% O₂, 5% CO₂)

Varying concentrations of each compound to be tested (in DMSO) were diluted in assay buffer [HBSS with Calcium/Magnesium (Lonza/BioWhittaker cat #10-527F)/0.1% BSA (Sigma Aldrich cat #A7409-1L)/20 mM HEPES (Lonza/BioWhittaker cat #17-737E)] containing 100 μM 3-isobutyl-1-methylxanthin (IBMX; Sigma cat #15879). The final DMSO concentration in the compound/assay buffer mixture is 1%.

After 48 hours, the growth media was removed from the assay plate wells, and the cells were treated with 20 μL of the serially diluted compound in assay buffer for 30 minutes at 37° C. in a humidified environment (95% O₂, 5% CO₂). Following the 30 minute incubation, 10 μL of labeled d2 cAMP and 10 μL of anti-CAMP antibody (both diluted 1:20 in cell lysis buffer; as described in the manufacturer's assay protocol) were added to each well of the assay plate. The plates were then incubated at room temperature and after 60 minutes, changes in the HTRF signal were read with an Envision 2104 multi-label plate reader using excitation of 330 nm and emissions of 615 and 665 nm. Raw data were converted to nM CAMP by interpolation from a cAMP standard curve (as described in the manufacturer's assay protocol) and the percent effect was determined relative to a saturating concentration of the full agonist GLP-1 (1 μM) included on each plate. EC₅₀ determinations were made from agonist dose response curves analyzed with a curve fitting program using a 4-parameter logistic dose response equation.

In Table BB-1, assay data are presented to two (2) significant figures as the geometric mean (EC₅₀S) and arithmetic mean (Emax) based on the number of replicates listed (Number).

TABLE BB-1
Biological activity for C111

| | | Assay 1 | | Assay 2 | Assay 2 | |
|---|---|---|---|---|---|---|
| | Assay 1 | Emax | Assay 1 | EC₅₀ | Emax | Assay 2 |
| Compound | EC₅₀ (nM) | (%) | Number | (nM) | (%) | Number |
| C111 | 1.1 | 79 | 5 | 13 | 100 | 18 |

All patents, patent applications and references referred to herein are hereby incorporated by reference in their entirety.

1. A process for preparing bis(4-methylbenzenesulfonate) salt of 3-fluoro-4-(((6-(piperidin-4-yl) pyridin-2-yl)oxy)methyl)benzonitrile, 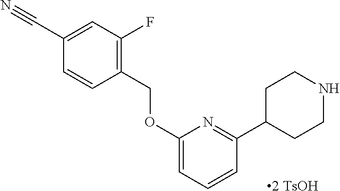
which process comprising:
(a1) reacting tert-butyl 4-(6-chloropyridin-2-yl) piperidine-1-carboxylate with 3-fluoro-4-(hydroxymethyl)benzonitrile in the presence a palladium catalyst, a base, and a phosphorous ligand, in a solvent system, to form tert-butyl 4-(6-((4-cyano-2-fluorobenzyl)oxy)pyridin-2-yl) piperidine-1-carboxylate, wherein the amount of the 3-fluoro-4-(hydroxymethyl)benzonitrile is about 1.0 to about 1.1 molar equivalents to the tert-butyl 4-(6-chloropyridin-2-yl) piperidine-1-carboxylate;
(a2) adding ethanol to the reaction mixture from Step (a1);
(a3) filtering the resultant reaction mixture from Step (a2);
(b1) adding p-toluenesulfonic acid monohydrate to the filtrate from Step (a3), thereby reacting the tert-butyl 4-(6-((4-cyano-2-fluorobenzyl)oxy)pyridin-2-yl) piperidine-1-carboxylate with the p-toluenesulfonic acid monohydrate, to form bis(4-methylbenzenesulfonate) salt of 3-fluoro-4-(((6-(piperidin-4-yl) pyridin-2-yl)oxy)methyl)benzonitrile, wherein the amount of the p-toluenesulfonic acid monohydrate is about 2.0 to about 3.0 molar equivalents to the tert-butyl 4-(6-((4-cyano-2-fluorobenzyl)oxy)pyridin-2-yl) piperidine-1-carboxylate; and
(b2) isolating the bis(4-methylbenzenesulfonate) salt of 3-fluoro-4-(((6-(piperidin-4-yl) pyridin-2-yl)oxy)methyl)benzonitrile from Step (b1).

2. A process for preparing bis(4-methylbenzenesulfonate) salt of 3-fluoro-4-(((6-(piperidin-4-yl) pyridin-2-yl)oxy)methyl)benzonitrile, 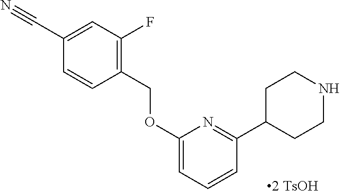
which process comprising:
(a1) reacting tert-butyl 4-(6-chloropyridin-2-yl) piperidine-1-carboxylate with 3-fluoro-4-(hydroxymethyl)benzonitrile in the presence a palladium catalyst, a base, and a phosphorous ligand, in a solvent system, to form the tert-butyl 4-(6-((4-cyano-2-fluorobenzyl)oxy)pyridin-2-yl) piperidine-1-carboxylate, wherein the amount of the 3-fluoro-4-(hydroxymethyl)benzonitrile is about 1.0 to about 1.1 molar equivalents to the tert-butyl 4-(6-chloropyridin-2-yl) piperidine-1-carboxylate;
(a2) upon reaction completion in Step (a1), adding water, ethyl acetate, and ethanol to the reaction mixture;
(a3) separating the organic phase from Step (a2) from the aqueous phase;
(b1) adding p-toluenesulfonic acid monohydrate to the separated organic phase from Step (a3), thereby reacting the tert-butyl 4-(6-((4-cyano-2-fluorobenzyl)oxy)pyridin-2-yl) piperidine-1-carboxylate with the p-toluenesulfonic acid monohydrate, to form bis(4-methylbenzenesulfonate) salt of 3-fluoro-4-(((6-(piperidin-4-yl) pyridin-2-yl)oxy)methyl)benzonitrile, wherein the amount of the p-toluenesulfonic acid monohydrate is about 2.0 to about 3.0 molar equivalents to the tert-butyl 4-(6-((4-cyano-2-fluorobenzyl)oxy)pyridin-2-yl) piperidine-1-carboxylate; and
(b2) isolating the bis(4-methylbenzenesulfonate) salt of 3-fluoro-4-(((6-(piperidin-4-yl) pyridin-2-yl)oxy)methyl)benzonitrile from Step (b1).

3. A process for preparing bis(4-methylbenzenesulfonate) salt of 3-fluoro-4-(((6-(piperidin-4-yl) pyridin-2-yl)oxy)methyl)benzonitrile, 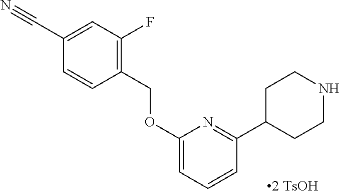
which process comprising:
(a1) reacting tert-butyl 4-(6-chloropyridin-2-yl) piperidine-1-carboxylate with 3-fluoro-4-(hydroxymethyl)benzonitrile in the presence of a copper catalyst, and a ligand, in a solvent system, to form tert-butyl 4-(6-((4-cyano-2-fluorobenzyl)oxy)pyridin-2-yl) piperidine-1-carboxylate, wherein the amount of the 3-fluoro-4-(hydroxymethyl)benzonitrile is about 1.1 to about 1.3 (e.g. 1.2) molar equivalents to the tert-butyl 4-(6-chloropyridin-2-yl) piperidine-1-carboxylate;
(a2) filtering the reaction mixture from Step (a1), wherein the filtering further comprises washing with methyl tert-butyl ether (MTBE);
(a3) concentrating the filtrate from Step (2) to obtain the tert-butyl 4-(6-((4-cyano-2-fluorobenzyl)oxy)pyridin-2-yl) piperidine-1-carboxylate;
(b1) dissolving the tert-butyl 4-(6-((4-cyano-2-fluorobenzyl)oxy)pyridin-2-yl) piperidine-1-carboxylate from Step (a3) in a solvent system comprising 1,4-dioxane and MTBE to form a solution; and then adding p-toluenesulfonic acid monohydrate to the solution, thereby reacting the tert-butyl 4-(6-((4-cyano-2-fluorobenzyl)oxy)pyridin-2-yl) piperidine-1-carboxylate with the p-toluenesulfonic acid monohydrate, to form bis(4-methylbenzenesulfonate) salt of 3-fluoro-4-(((6-(piperidin-4-yl) pyridin-2-yl)oxy)methyl)benzonitrile, wherein the amount of the p-toluenesulfonic acid monohydrate is about 2.0 to about 2.5 molar equivalents to the tert-butyl 4-(6-((4-cyano-2-fluorobenzyl)oxy)pyridin-2-yl) piperidine-1-carboxylate; and
(b2) isolating the bis(4-methylbenzenesulfonate) salt of 3-fluoro-4-(((6-(piperidin-4-yl) pyridin-2-yl)oxy)methyl)benzonitrile from Step (b1).

4-12. (canceled)

13. The process of claim 1, wherein the amount of the palladium catalyst is about 0.1 molar % to about 2.0 molar % of the tert-butyl 4-(6-chloropyridin-2-yl) piperidine-1-carboxylate in Step (a1).

14. The process of claim 1, wherein the palladium catalyst is palladium (II) acetate.

15. The process of claim 1, wherein the amount of the phosphorous ligand is about 2.0 molar equivalent to the palladium catalyst in Step (a1).

16. The process of claim 1, wherein the amount of the base is about 1 to about 2 molar equivalents to the tert-butyl 4-(6-chloropyridin-2-yl) piperidine-1-carboxylate in Step (a1).

17. The process of claim 1, wherein the volume amount of the anisole is about 5 ml/g to about 10 ml/g based on the weight of the tert-butyl 4-(6-chloropyridin-2-yl) piperidine-1-carboxylate in Step (a1).

18. The process of claim 1, wherein the reaction mixture is stirred at about 80 to about 120° C. for a for a time sufficient to form tert-butyl 4-(6-((4-cyano-2-fluorobenzyl)oxy)pyridin-2-yl) piperidine-1-carboxylate in Step (a1).

19. The process of claim 1, wherein the reaction mixture is cooled down to the ambient temperature after reaction completion in Step (a1) and before Step (a2) is carried out.

20. The process of claim 1, wherein the volume amount of the ethanol in Step (a2) is about 0.2 to about 0.3 equivalents of the volume amount of the anisole in Step (a1).

21. The process of claim 1, wherein the filtering in Step (a3) further comprises washing with ethyl acetate. In some further embodiment, the volume amount of the ethyl acetate used to wash is about 0.4 to about 0.6 equivalent of the volume amount of the anisole in Step (a1).

22. The process of claim 1, wherein the amount of the p-toluenesulfonic acid monohydrate is about 2.0 to about 2.5 molar equivalents to the tert-butyl 4-(6-((4-cyano-2-fluorobenzyl)oxy)pyridin-2-yl) piperidine-1-carboxylate in Step (b1).

23. The process of claim 1, wherein the amount of the p-toluenesulfonic acid monohydrate is about 2.1 to about 2.4 molar equivalents to the tert-butyl 4-(6-((4-cyano-2-fluorobenzyl)oxy)pyridin-2-yl) piperidine-1-carboxylate in Step (b1).

24. The process of claim 1, wherein reaction mixture in Step (b1) is stirred at about 30° C. to about 60° C. for a for a time sufficient to form bis(4-methylbenzenesulfonate) salt of 3-fluoro-4-(((6-(piperidin-4-yl) pyridin-2-yl)oxy)methyl)benzonitrile.

25. The process of claim 1, wherein the p-toluenesulfonic acid monohydrate, as a neat reagent (i.e., without premixing with a solvent), is added to reaction mixture in Step (b1).

26. The process of claim 1, wherein isolating the bis(4-methylbenzenesulfonate) salt of 3-fluoro-4-(((6-(piperidin-4-yl) pyridin-2-yl)oxy)methyl)benzonitrile in Step (b2) comprises cooling the reaction mixture and filtering the mixture after the reaction is complete from Step (b1).

27. The process of claim 26, wherein filtering the mixture further comprises washing the solid obtained by the filtration with anisole.

28. The process of claim 1, wherein the isolated bis(4-methylbenzenesulfonate) salt of 3-fluoro-4-(((6-(piperidin-4-yl) pyridin-2-yl)oxy)methyl)benzonitrile in Step (b2) is further dried, optionally under vacuum.

29. The process of claim 1, wherein the bis(4-methylbenzenesulfonate) salt of 3-fluoro-4-(((6-(piperidin-4-yl) pyridin-2-yl)oxy)methyl)benzonitrile isolated in Step (b2) is an anhydrous form.
